SEQUENCE LISTING (4 sequences)

SEQ ID NO: 1 - DNA, 20 bp, Artificial sequence
  Single strand DNA oligonucleotide
taatacgactcactataggg

SEQ ID NO: 2 - DNA, 20 bp, Artificial sequence
  Single strand DNA oligonucleotide
ggaaacagctatgaccatga

SEQ ID NO: 3 - DNA, 20 bp, Artificial sequence
  Single strand DNA oligonucleotide
caccagactgactcctcatt

SEQ ID NO: 4 - DNA, 20 bp, Artificial sequence
  Single strand DNA oligonucleotide
cctgttgctgcacatattcc

(19) United States
(12) Patent Application Publication
(10) Pub. No.: US 2011/0178182 A1
(43) Pub. Date: Jul. 21, 2011

(54) ENRICHED NANOSTRUCTURE COMPOSITION

(75) Inventor: Eran Gabbai, Kfar-MaAs (IL)

(73) Assignee: DO-COOP TECHNOLOGIES LTD.

(21) Appl. No.: 12/674,339

(22) PCT Filed: Aug. 20, 2008
(86) PCT No.: PCT/IL2008/001142

§ 371 Date: Feb. 19, 2010

Related U.S. Application Data
(60) Provisional application No. 61/196,692, filed on Aug. 20, 2007.

(51) Int. Cl. A61K 47/02; C12N 9/12; C12P 19/34; C12Q 1/68; A01N 25/00; C12N 15/02; C12N 5/07; C12P 21/08; A01N 1/00; C09K 3/00; H01F 1/00; C12N 1/20; B82Y 30/00; B82Y 5/00; B82Y 15/00

(52) U.S. Cl. 514/770; 435/194; 435/91.2; 435/6.12; 435/449; 435/325; 435/404; 435/70.21; 435/1.3; 252/182.32; 252/62.51 R; 435/253.6; 977/773; 977/902; 977/915; 977/926

(57) ABSTRACT

A method of producing a nanostructure composition from a solid powder is disclosed. The method comprises: (a) heating the solid powder, thereby providing a heated solid powder; (b) immersing the heated solid powder in a liquid in the presence of a gas medium, the liquid being colder than the heated powder, and (c) irradiating the cold liquid, the heated solid powder and the gas medium by electromagnetic radiation selected such that nanostructures are formed from particles of the solid powder and a stable gas phase is formed from the gas medium.

### FIELD AND BACKGROUND OF THE INVENTION

The present invention, in some embodiments thereof, relates to nanostructures and, more particularly, but not exclusively, to an enriched nanostructure composition.

Nanoscience is the science of small particles of materials and is one of the most important research frontiers in modern science. These small particles are of interest from a fundamental view point since all properties of a material, such as its melting point and its electronic and optical properties, change when the of the particles that make up the material become nanoscopic.

In the biotechnology area, for example, nanoparticles are frequently used in nanometer-scale equipment for probing the real-space structure and function of biological molecules. Auxiliary nanoparticles, such as calcium alginate nanospheres, have also been used to help improve gene transfection protocols.

Traditionally, nanoparticles are synthesized from a molecular level up, by the application of arc discharge, laser evaporation, pyrolysis process, use of plasma, use of sol gel and the like. Widely used nanoparticles are the fullerene carbon nanotubes, which are broadly defined as objects having a diameter below about 1 μm. In a narrower sense of the words, a material having the carbon hexagonal mesh sheet of carbon substantially in parallel with the axis is called a carbon nanotube, and one with amorphous carbon surrounding a carbon nanotube is also included within the category of carbon nanotube.

Also known in the art are nanoshells which are nanoparticles having a dielectric core and a conducting shell layer. Similar to carbon nanotubes, nanoshells are also manufactured from a molecular level up, for example, by bonding atoms of metal on a dielectric substrate. Nanoshells are particularly useful in applications in which it is desired to exploit the above mention optical field enhancement phenomenon.

International Patent Publication Nos. WO2003/053647 and WO2005/079153 to Gabbai disclose a top down process for the preparation of solid-fluid composition. A raw powder of micro-sized particles is heated to high temperature and is subsequently immersed into cold water under condition of electromagnetic radiofrequency (RF) radiation. The combination of cold water and RF radiation influences the interface between the particles and the water, and breaks both the water molecules and the particles. The broken water molecules are in the form of free radicals, which envelope the debris of the particles.

Gabbai's composition has been utilized for many applications, particularly in the field of life sciences. To this end see International Patent Publication Nos. WO2007/077562, WO2007/077560, WO2007/077561, WO2007/077563, WO2008/081456 and WO2008/081455, the contents of which are hereby incorporated by reference.

Heretofore, Gabbai's composition has been utilized for de-folding DNA molecules, altering bacterial adherence to biomaterial, stabilizing enzyme activity, improving affinity binding of nucleic acids to a resin and improving gel electrophoresis separation, increasing a capacity of a column, improving efficiency of nucleic acid amplification process, manipulating macromolecules in the presence of a solid support, enhancing in vivo uptake of a pharmaceutical agent into a cell, culturing of eukaryotic cells and generating monoclonal antibodies. Gabbai's composition has also been utilized for buffering, cell-fusion, analyte detection, disinfection, sterilization and cryoprotection.

### SUMMARY OF THE INVENTION

According to an aspect of some embodiments of the present invention there is provided a method of producing a nanostructure composition from a solid powder, the method comprises: (a) heating the solid powder, thereby providing a heated solid powder; (b) immersing the heated solid powder in a liquid in the presence of a gas medium, the liquid being colder than the heated powder; and (c) irradiating the cold liquid, the heated solid powder and the gas medium by electromagnetic radiation selected such that nanostructures are formed from particles of the solid powder and a stable gas phase is formed from the gas medium.

According to some embodiments of the invention the method further comprises passing the heated solid powder through the gas medium prior to the immersion so as to establish the presence of the gas medium.

According to some embodiments of the invention the method further comprises introducing the gas medium into the liquid prior to the immersion so as to establish the presence of the gas medium.

According to some embodiments of the invention the gas medium comprises a hydrophobic gas. According to some embodiments of the invention the gas medium is selected from the group consisting of carbon dioxide, oxygen, nitrogen, sulfur dioxide, hydrogen, fluorine, methane, hexane, hexafluoroethane and air.

According to some embodiments of the invention the solid powder comprises micro-sized particles. According to some embodiments of the invention the micro-sized particles are crystalline particles.

According to some embodiments of the invention the nanostructures are crystalline nanostructures.

According to some embodiments of the invention the liquid comprises water.

According to some embodiments of the invention the solid powder is selected from the group consisting of a ferroelectric material and a ferromagnetic material. According to some embodiments of the invention the solid powder is selected from the group consisting of BaTiO₃, WO₃, Ba₂F₉O₁₂ and BaSO₄.

According to some embodiments of the invention the solid powder comprises hydroxyapatite.

According to some embodiments of the invention the solid powder comprises a material selected from the group consisting of a mineral, a ceramic material, glass, metal and synthetic polymer.

According to some embodiments of the invention the electromagnetic radiation is in the radiofrequency range.

According to some embodiments of the invention the electromagnetic radiation is continues wave electromagnetic radiation.

According to some embodiments of the invention the electromagnetic radiation is modulated electromagnetic radiation.

According to an aspect of some embodiments of the present invention there is provided a nanostructure composition. The nanostructure composition comprises a liquid, nanostructures and a stable or meta-stable gas phase, wherein at least one of the nanostructures has a core material of a nanometric size and an envelope of ordered fluid molecules being in a steady physical state with the core material.

According to some embodiments of the present invention the nanostructure composition is capable of releasing the gas in response to excitation energy applied thereto and collecting the gas when the excitation energy is terminated.

According to some embodiments of the present invention the nanostructure composition is prepared in non-atmospheric conditions.

According to some embodiments of the present invention the nanostructure composition is prepared in the presence of a gas jet.

According to some embodiments of the present invention the nanostructure composition is prepared in the presence of gas at a concentration which is substantially different from natural atmospheric concentration of the gas.

According to some embodiments of the present invention the nanostructure composition is prepared in the presence of gas at a temperature which is substantially below an ambient temperature.

According to some embodiments of the present invention the envelope of fluid molecules is distinguishable from the liquid.

According to some embodiments of the present invention the core material is crystalline.

According to some embodiments of the present invention the liquid comprises water.

According to some embodiments of the present invention the gas phase comprises a hydrophobic gas.

According to some embodiments of the present invention the gas phase is selected from the group consisting of carbon dioxide, oxygen, nitrogen, sulfur dioxide, hydrogen, fluorine, methane, hexane, hexafluoroethane and air.

According to some embodiments of the present invention the gas phase resides in or attached to the envelope.

According to some embodiments of the present invention the gas phase resides in or attached to the core.

According to some embodiments of the present invention the gas phase resides in liquid regions between the nanostructures.

According to some embodiments of the present invention the nanostructure composition has the property that when it is contacted with a surface and then washed off the surface by a predetermined wash protocol, an electrochemical signature of the composition is preserved on the surface.

According to some embodiments of the present invention the nanostructure composition is characterized by a zeta potential which is substantial larger than a zeta potential of the liquid per se.

According to some embodiments of the present invention the nanostructure has a specific gravity which is lower than or equal to a specific gravity of the liquid.

According to some embodiments of the present invention the nanostructure composition is capable of changing spectral properties of a dyed solution.

According to some embodiments of the present invention the nanostructure composition is characterized by an enhanced ultrasonic velocity relative to water.

According to some embodiments of the present invention the nanostructure composition is capable of facilitating increment of bacterial colony expansion rate.

According to some embodiments of the present invention the nanostructure composition is capable of facilitating increment of phage-bacteria or virus-cell interaction.

According to some embodiments of the present invention the nanostructure composition is capable of enhancing macromolecule binding to solid phase matrix.

According to some embodiments of the present invention the nanostructure composition is capable of at least partially de-folding DNA molecules.

According to some embodiments of the present invention the nanostructure composition is capable of stabilizing enzyme activity.

According to some embodiments of the present invention the nanostructure composition is capable of altering bacterial adherence to biomaterial.

According to some embodiments of the present invention the nanostructure composition is capable of improving affinity binding of nucleic acids to a resin and improving gel electrophoresis separation.

According to some embodiments of the present invention the nanostructure composition is capable of increasing a capacity of a column.

According to some embodiments of the present invention the nanostructure composition is characterized by an enhanced ability to dissolve or disperse a substance relative to water.

According to some embodiments of the present invention the nanostructure composition is characterized by an enhanced buffering capacity relative to water.

According to some embodiments of the present invention the nanostructure composition is capable of improving efficiency of nucleic acid amplification process.

According to an aspect of some embodiments of the present invention there is provided a kit for polymerase chain reaction. The kit comprises, in separate packaging: (a) a thermostable DNA polymerase; and (b) the nanostructure composition described herein.

According to an aspect of some embodiments of the present invention there is provided a method of amplifying a DNA sequence, the method comprises: (a) providing the nanostructure composition described herein; and (b) in the presence of the nanostructure composition, executing a plurality of polymerase chain reaction cycles on the DNA sequence, thereby amplifying the DNA sequence.

According to some embodiments of the present invention the nanostructure composition is capable of improving efficiency of real-time polymerase chain reaction.

According to an aspect of some embodiments of the present invention there is provided a kit for real-time polymerase chain reaction, the kit comprises: (a) a thermostable DNA polymerase; (b) a double-stranded DNA detecting molecule; and (c) the nanostructure composition described herein.

According to some embodiments of the present invention the nanostructure composition is capable of allowing the manipulation of at least one macromolecule in the presence of a solid support.

According to an aspect of some embodiments of the present invention there is provided an antiseptic composition, the antiseptic composition comprises at least one antiseptic agent and the enriched nanostructure composition described herein.

According to an aspect of some embodiments of the present invention there is provided a method of disinfecting a body surface of an individual, the method comprises providing to an individual in need thereof an antiseptic effective amount of a composition wherein the composition comprises the nanostructure composition described herein, thereby disinfecting a body surface of an individual.

According to an aspect of some embodiments of the present invention there is provided a method of sterilizing an object, the method comprises contacting the object with a composition which comprises the nanostructure composition described herein, thereby sterilizing the object.

According to an aspect of some embodiments of the present invention there is provided a cryoprotective composition, the cryoprotective composition comprises the nanostructure composition of described herein, and at least one cryoprotective agent.

According to an aspect of some embodiments of the present invention there is provided a method of cryopreserving cellular matter, the method comprises (a) contacting the cellular matter with the nanostructure composition described herein; and (b) subjecting the cellular matter to a cryopreserving temperature, thereby cryopreserving the cellular matter.

According to an aspect of some embodiments of the present invention there is provided a cryopreservation container which comprises the cryoprotective composition.

According to an aspect of some embodiments of the present invention there is provided a pharmaceutical composition which comprises: (a) at least one pharmaceutical agent as an active ingredient; (b) the nanostructure composition as described herein, wherein the nanostructure composition is formulated to enhance in vivo uptake of the at least one pharmaceutical agent.

According to an aspect of some embodiments of the present invention there is provided a method of enhancing in vivo uptake of a pharmaceutical agent into a cell, the method comprises administering the pharmaceutical composition to an individual, thereby enhancing in vivo uptake of the pharmaceutical agent into the cell.

According to an aspect of some embodiments of the present invention there is provided a method of cell-fusion, the method comprises fusing cells in a medium comprises the nanostructure composition described herein, thereby fusing cells.

According to an aspect of some embodiments of the present invention there is provided a method of culturing eukaryotic cells, the method comprises incubating the cells in a medium comprises the nanostructure composition described herein, thereby culturing eukaryotic cells.

According to an aspect of some embodiments of the present invention there is provided a cell culture medium comprises a eukaryotic cell culture medium and the nanostructure composition described herein.

According to an aspect of some embodiments of the present invention there is provided an article of manufacture comprises packaging material and a nanostructure composition identified for the culturing of eukaryotic cells being contained within the packaging material, the nanostructure composition comprises the nanostructure composition described herein.

According to an aspect of some embodiments of the present invention there is provided an article of manufacture comprises packaging material and a nanostructure composition identified for generating monoclonal antibodies being contained within the packaging material, the nanostructure composition comprises the nanostructure composition described herein.

According to an aspect of some embodiments of the present invention there is provided a method of generating a monoclonal antibody, the method comprises fusing an immortalizing cell with an antibody producing cell to obtain a hybridoma in a medium comprises the nanostructure composition described herein.

According to an aspect of some embodiments of the present invention there is provided a method of dissolving or dispersing cephalosporin. The method comprises contacting the cephalosporin with the nanostructure composition described herein under conditions allowing dispersion or dissolving of the substance.

According to an aspect of some embodiments of the present invention there is provided a kit for detecting an analyte. The kit comprises: (a) a detectable agent; and (b) the nanostructure composition described herein.

According to an aspect of some embodiments of the present invention there is provided an article of manufacture. The article of manufacture comprises packaging material and a nanostructure composition identified for enhancing detection of a detectable moiety being contained within the packaging material, the composition comprises the nanostructure composition described herein.

According to an aspect of some embodiments of the present invention there is provided apparatus for recycling gas. The apparatus comprises a nanostructure composition and an excitation device for exciting the nanostructure composition, the nanostructure composition being capable of releasing gas when the excitation device is active, and collecting the gas upon deactivation of the excitation device.

According to an aspect of some embodiments of the present invention there is provided a method of attracting insects. The method comprises activating the excitation device of the apparatus, thereby attracting the insects.

According to an aspect of some embodiments of the present invention there is provided a method of enhancing plant growth. The method comprises activating the excitation device during daylight hours, thereby enhancing plant growth.

According to an aspect of some embodiments of the present invention there is provided an apparatus for recycling carbon dioxide. The apparatus comprises a composition and an excitation device for exciting the composition. In various exemplary embodiments of the invention the composition is capable of releasing carbon dioxide when the excitation device is active, and collecting carbon dioxide upon deactivation of the excitation device.

According to some embodiments of the present invention the apparatus is incorporated in an apparatus for attracting insects. Thus, the excitation device of the apparatus can be activated so as to attracting insects.

According to some embodiments of the present invention the apparatus is incorporated in an apparatus for enhancing plant growth. Thus, the excitation device of the apparatus can be activated so as to enhance plant growth.

According to some embodiments of the present invention the apparatus further comprises a gas separating mechanism designed and constructed to separate gases contacting the composition from carbon dioxide released from the composition.

According to some embodiments of the present invention the apparatus further comprises a chamber for holding the composition wherein the gas separating mechanism comprises a sleeve extending from the environment into the composition.

According to some embodiments of the present invention the apparatus further comprises an outlet valve positioned in the sleeve and configured for controlling the release of the carbon dioxide to the environment.

According to some embodiments of the present invention the apparatus further comprises an inlet valve positioned at a wall of the chamber and configured for controlling fluid communication between a surface of the composition and the environment.

According to an aspect of some embodiments of the present invention there is provided apparatus for trapping insects. The apparatus comprises: a chamber containing a composition having at least a stable state and an excited state. The excited state is characterized by efflux of carbon dioxide through an outlet of the chamber, and a transition from the excited state to the stable state is accompanied by an influx of carbon dioxide through an inlet of the chamber. In various exemplary embodiments of the invention the apparatus further comprises an excitation device for exciting the composition, and an insect trap device designed and configured to trap insects being nearby the outlet.

According to an aspect of some embodiments of the present invention there is provided a method of trapping insects. The method comprises operating the insect trapping apparatus, thereby trapping the insects.

According to some embodiments of the present invention the excitation device comprises a radiofrequency transmitter.

According to some embodiments of the present invention the composition in the apparatus is the enriched nanostructure composition described above.

According to some embodiments of the present invention the apparatus further comprises a sleeve extending from the outlet into the composition.

According to some embodiments of the present invention the apparatus further comprises an outlet valve positioned in the sleeve and configured for controlling the efflux of the carbon dioxide.

According to some embodiments of the present invention the apparatus further comprises an inlet valve positioned at the inlet and configured for controlling the influx.

According to some embodiments of the present invention the apparatus further comprises a control unit configured for activating and deactivating the excitation device.

According to some embodiments of the present invention the control unit is configured for intermittently activating the excitation device.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a gas enriched nanostructure composition, according to various exemplary embodiments of the present invention;

FIG. 2 is a schematic illustration of gas recycling process, according to various exemplary embodiments of the present invention;

FIG. 3 is a flowchart diagram of a method for fabricating an enriched composition, according to various exemplary embodiments of the present invention;

FIG. 4 is a schematic illustration of a system for fabricating an enriched composition, according to various exemplary embodiments of the present invention;

FIG. 5 which is a schematic illustration of an apparatus for recycling a gas, according to various exemplary embodiments of the present invention;

FIG. 6 is a schematic illustration of apparatus for trapping insects, according to various exemplary embodiments of the present invention;

FIG. 7 is a schematic illustration of electrochemical deposition (ECD) experimental setup, used in experiments performed by the present inventor;

FIG. 8 are images showing ECD scores, used according to some embodiments of the present invention to define ECD patterns obtained in experiments performed by the present inventor;

FIG. 9 is a graph showing conductivity as a function of the inorganic carbon (IC) content as obtained in experiments performed by the present inventor;

FIG. 10 shows weight losses as a result of heating of carbon dioxide enriched nanostructure compositions manufactured according to various exemplary embodiments of the present invention;

FIG. 11 shows IC content as measure before heating, immediately following heating, and one week after heating of carbon dioxide enriched nanostructure compositions manufactured according to various exemplary embodiments of the present invention;

FIG. 12 show pH values as obtained before heating and one week after heating of carbon dioxide enriched nanostructure compositions manufactured according to various exemplary embodiments of the present invention;

FIG. 13 is an image of a prototype apparatus produced according to various exemplary embodiments of the present invention; and

FIGS. 14-53 are plots of carbon dioxide concentration levels as a function of time, as measured for three prototype apparatus produced according to various exemplary embodiments of the present invention.

### DESCRIPTION OF SPECIFIC EMBODIMENTS OF THE INVENTION

The present invention, in some embodiments thereof, relates to nanostructures and, more particularly, but not exclusively, to an enriched nanostructure composition.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present inventor has discovered that a composition containing nanostructures can be enriched with gas such that the gas remains in the composition. Such composition is referred to herein as a gas enriched nanostructure composition abbreviated GENC. A composition suitable for the present embodiments can be a nanostructure composition in which there is a stable or meta-stable gas phase.

A stable gas phase as used herein refers to a gas phase which remains in the liquid for a prolong period of time (from several days to several years, e.g., several months) without being spontaneously released to the environment.

A meta-stable gas phase is referred to refers to a gas phase which is spontaneously released from the liquid to the environment after a relatively short period of time (from several minutes to several days, e.g., several hours).

Spontaneous release of gas refers to a gas release which occurs when the liquid is in thermal equilibrium with the environment without any application of energy.

As demonstrated in the Examples section that follows, a nanostructure composition which comprises liquid and nanostructures contains a stable gas phase. Without being bound to a specific theory, the present inventor postulates that stable gas phase of the composition can reside near the nanostructures or in spaces formed between the nanostructures. Such stable gas phase is typically in the form of nanobubbles. Yet, it is not excluded that the gas phase is trapped in a crystalline core of the nanostructures.

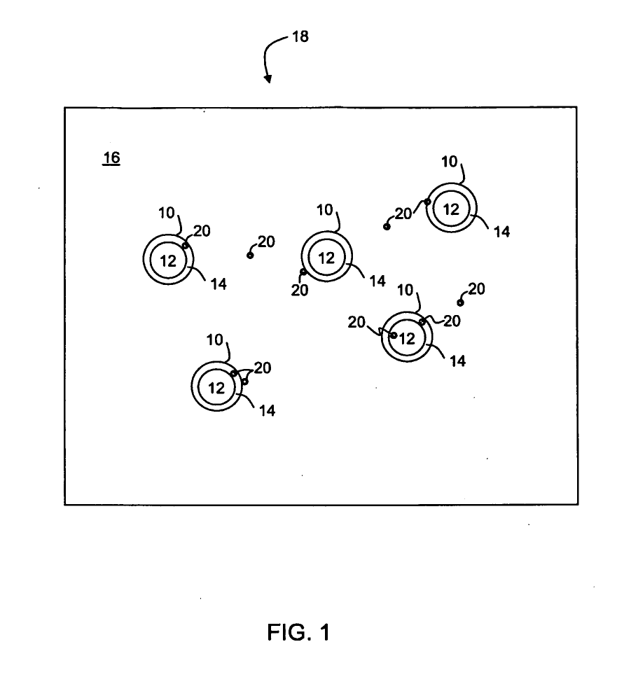

FIG. 1 is a schematic illustration of a gas enriched nanostructure composition 18, according to various exemplary embodiments of the present invention. Composition 18 comprises a nanostructure 10 and a liquid 16, such as, but not limited to, an aquatic, e.g., water.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, in which case the nanostructure is referred to as a “continuous nanostructure”, or between several hundreds of picometers to several hundreds of nanometers, in which the nanostructure is referred to as a “discontinuous nanostructure”. Thus, the nanostructure of the present embodiments can comprise a nanoparticle, an arrangement of nanoparticles, or any arrangement of one or more nanoparticles and one or more molecules.

According to an embodiment of the present invention nanostructure 10 comprises a core material 12 of a nanometric size, surrounded by an envelope 14 of ordered fluid molecules, which are typically in gaseous state. Core material 12 and envelope 14 are preferably in a steady physical state. In the present context, “steady physical state” referrers to a situation in which the core material and the envelope are bound by any potential having at least a local minimum.

As used herein the phrase “ordered fluid molecules” is referred to an organized arrangement of fluid molecules having correlations thereamongst.

In various exemplary embodiments of the invention envelope 14 is distinguishable from core 12. This can be validated, for example, using cryogenic-temperature transmission electron microscopy, as further detailed in the examples section that follows.

Composition 18 further comprises a gas phase 20, which resides in or attached to at least one of: envelope 14, core 12 and liquid 16. More specifically, gas phase 20 can (i) reside in envelope 14, in which case the ordered fluid molecules also envelope gas phase 20; (ii) reside in the boundary between nanostructure 10 and liquid 16, in which case gas phase 20 is attached (e.g., bound) to envelope 14, but is not surrounded thereby; (iii) reside in the solid structure (e.g., crystalline structure) of core material 12, e.g., in which case gas phase 20 is trapped between atoms or molecules of core material 12; and/or (iv) reside in liquid regions between nanostructures 10. Any combination of these relations is also contemplated.

Gas phase 20 can be in the form of gas molecules or is can be in a form of larger objects, such as, but not limited to, nanobubbles, which contain two or more gas molecules.

Many types of gasses are suitable for composition 18. Typically, the gas is hydrophobic. Representative examples of suitable gases include, without limitation, carbon dioxide (CO₂), oxygen (O), nitrogen (N), sulfur dioxide (SO₂), hydrogen (H), fluorine (F), methane (CH₄), hexane (C₆H₁₄), hexafluoroethane (C₂F₆) and air. In various exemplary embodiments of the invention the gas is carbon dioxide (CO₂). As demonstrated below, in case of a CO₂ gas phase, the enriched composition of the present embodiments still has a relatively high pH values, indicating that the gas phase is not dissolved in the liquid.

The present inventor has discovered that an efflux of the gas can be generated by exciting the enriched nanostructure composition of the present embodiments to an excited state. The present inventor has uncovered that when the composition experience a transition from an excited state to a stable state, the composition act as a sink material for the gas. The present inventor has therefore uncovered that an influx of the gas can be generated by allowing excited compositions to return to their stable state.

Thus, in various exemplary embodiments of the invention the enriched nanostructure composition is characterized by at least a stable state and an excited state. The excited state is characterized by efflux of the gas from the composition, and a transition from the excited state to the stable state is accompanied by an influx of the gas to the composition. These embodiments are particularly useful when the composition is used for gas recycling. For example, In some embodiments of the present invention the enriched nanostructure composition is used for recycling CO2.

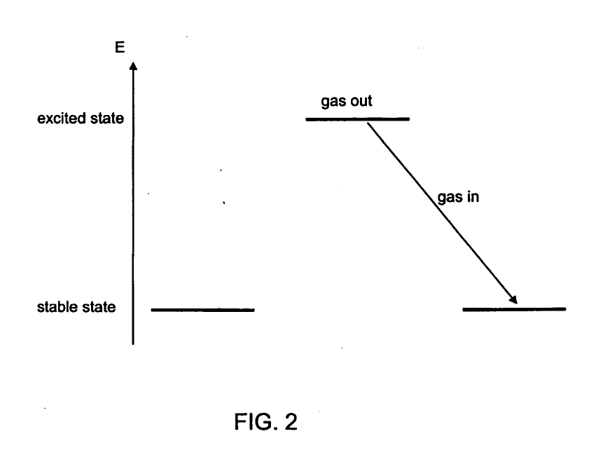

The gas recycling process is illustrated in FIG. 2. As shown, the composition of the present embodiments generally has a stable state and an excited state. The term “generally” in the present context implies that the composition can have more than two states. For example, the composition may have one stable state and a plurality of excited states. The composition may also have a continuum of excited states. The term “excited state” in the present context refers to a state in which the energy E of the composition is higher that the energy when the composition is in its stable state.

The term “stable state” in the present context refers to a state in which the composition remains for a prolong period of time, substantially without experiencing spontaneous macroscopic transitions to another state. Typically, in the absence of severe conditions (such as delivery of vast amount of energy to the composition), the composition of the present embodiments can remain in its stable state for at least a day, more preferably at least a week, more preferably at least a month, more preferably at least a year, more preferably at least a few years.

Preferably, but not obligatorily, the excited state (or states) of the composition are non-stable or meta-stable. When the excited state is non-stable, the transition from the excited state to the stable state is spontaneous, and there is no need to supply energy to the composition in order to achieve such transition. When the excited state is meta-stable, the transition from the excited state to the stable state can be achieved by supplying energy at a sufficient amount to perturb the composition to a non-stable state from which the composition spontaneously returns to the stable state.

Typically, the gas content of the composition is sufficiently high when the composition is in a stable state and is lower when the composition is in an excited state.

In various exemplary embodiments of the invention the composition is capable of generating a local concentration of gas, which is well above the normal ambient concentration. For example, in experiments performed by the present inventor (see the Examples section that follows), it was found that when the gas is carbon dioxide, the enriched nanostructure composition can produce CO₂ at a local concentration of the order of 1000 parts per million (ppm) by volume and more. In various exemplary embodiments of the invention the composition produces CO₂ at a local concentration of at least 2000 ppm by volume. Optionally and preferably, the composition produces CO₂ bursts at a local concentration of the order of 10,000 ppm by volume.

Core material 12 of composition 18 is not limited to a certain type or family of materials, and can be selected in accordance with the application for which the nanostructure is designed. Representative examples include, without limitation, ferroelectric material, a ferromagnetic material and a piezoelectric material. Also contemplated, is a core made of hydroxyapatite (HA). In some embodiments of the present invention core material 12 has a crystalline structure.

A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. According to some embodiments of the present invention, when core material 12 is ferroelectric or ferromagnetic, nanostructure 10 retains its ferroelectric or ferromagnetic properties. Hence, nanostructure 10 has a particular feature in which macro scale physical properties are brought into a nanoscale environment.

According to some embodiments of the present invention nanostructure 10 is capable of clustering with at least one additional nanostructure. More specifically, when a certain concentration of nanostructure 10 is mixed in a liquid (e.g., water), attractive electrostatic forces between several nanostructures may cause adherence thereamongst so as to form a cluster of nanostructures. Preferably, even when the distance between the nanostructures prevents cluster formation, nanostructure 10 is capable of maintaining long range interaction (about 0.5-10 μm), with the other nanostructures.

As used herein the term “about” refers to ±10%.

The unique properties of nanostructure 10 may be accomplished, for example, by producing nanostructure 10 using a “top-down” process. More specifically, nanostructure 10 can be produced from a raw powder of micro-sized particles, say, above 1 μm or above 10 μm in diameter, which are broken in a controlled manner, to provide nanometer-sized particles. Typically, such a process is performed in a cold liquid (preferably, but not obligatory, water) into which high-temperature raw powder is inserted, under condition of electromagnetic radiofrequency (RF) radiation.

Following is a review of the physical properties of water, which, as stated, can serve as the liquid in a composition comprising nanostructures.

Water is one of a remarkable substance, which has been very well studied. Although it appears to be a very simple molecule consisting of two hydrogen atoms attached to an oxygen atom, it has complex properties. Water has numerous special properties due to hydrogen bonding, such as high surface tension, high viscosity, and the capability of forming ordered hexagonal, pentagonal of dodecahedral water arrays by themselves of around other substances.

The melting point of water is over 100 K higher than expected when considering other molecules with similar molecular weight. In the hexagonal ice phase of the water (the normal form of ice and snow), all water molecules participate in four hydrogen bonds (two as donor and two as acceptor) and are held relatively static. In liquid water, some hydrogen bonds must be broken to allow the molecules move around. The large energy required for breaking these bonds must be supplied during the melting process and only a relatively minor amount of energy is reclaimed from the change in volume. The free energy change must be zero at the melting point. As temperature is increased, the amount of hydrogen bonding in liquid water decreases and its entropy increases. Melting will only occur when there is sufficient entropy change to provide the energy required for the bond breaking. The low entropy (high organization) of liquid water causes this melting point to be high.

Most of the water properties are attributed to the above mentioned hydrogen bonding occurring when an atom of hydrogen is attracted by rather strong forces to two oxygen atoms (as opposed to one), so that it can be considered to be acting as a bind between the two atoms.

Water has high density, which increases with the temperature, up to a local maximum occurring at a temperature of 3.984° C. This phenomenon is known as the density anomaly of water. The high density of liquid water is due mainly to the cohesive nature of the hydrogen-bonded network. This reduces the free volume and ensures a relatively high-density, compensating for the partial open nature of the hydrogen-bonded network. The anomalous temperature-density behavior of water can be explained utilizing the range of environments within whole or partially formed clusters with differing degrees of dodecahedral puckering.

The density maximum (and molar volume minimum) is brought about by the opposing effects of increasing temperature, causing both structural collapse that increases density and thermal expansion that lowers density. At lower temperatures, there is a higher concentration of expanded structures whereas at higher temperatures there is a higher concentration of collapsed structures and fragments, but the volume they occupy expands with temperature. The change from expanded structures to collapsed structures as the temperature rises is accompanied by positive changes in entropy and enthalpy due to the less ordered structure and greater hydrogen bond bending, respectively.

Generally, the hydrogen bonds of water create extensive networks, that can form numerous hexagonal, pentagonal of dodecahedral water arrays. The hydrogen-bonded network possesses a large extent of order. Additionally, there is temperature dependent competition between the ordering effects of hydrogen bonding and the disordering kinetic effects.

As known, water molecules can form ordered structures and superstructures. For example, shells of ordered water form around various biomolecules such as proteins and carbohydrates. The ordered water environment around these biomolecules are strongly involved in biological function with regards to intracellular function including, for example, signal transduction from receptors to cell nucleus. Additionally these water structures are stable and can protect the surface of the molecule.

Most of the ordered structure of liquefied water is on a short-range scale, typically about 1 nm. Although long-range order may, in principle exists, when the water is in its liquid phase, such long-range order has extremely low probability to occur spontaneously, because molecules in a liquid state are in constant thermal motion. Due to the hydrogen bonding and the non-bonding interactions, water molecules can form an infinite hydrogen-bonded network with specific and structured clustering. Thus, small clusters of water molecules can form water octamers that can further cluster with other smaller clusters to form icoshedral water clusters consisting of hundreds of water molecules. Therefore, water molecules can form ordered structures.

Other water properties include high boiling point, high critical point, reduction of the melting point with pressure (the pressure anomaly), compressibility which decreases with increasing temperature up to a minimum at about 46° C., and the like.

The unique properties of water have been exploited by the inventor of the present invention for the purpose of producing composition 18.

In various exemplary embodiments of the invention the enriched nanostructure composition is manufactured in non-atmospheric conditions. In some embodiments of the present invention, the enriched nanostructure composition is manufactured in the presence of gas which is not naturally present in the atmosphere and/or in the presence of gas whose concentration is substantially higher (e.g., at least two times higher, or at least ten time higher, or at least a hundred times higher) or substantially lower (e.g., at least two times lower, or at least ten time lower, or at least a hundred times lower) than its natural concentration.

For example, the enriched nanostructure composition of the present embodiments can be manufactured in the presence of carbon dioxide at a concentration which is above the atmospheric concentration of carbon dioxide. The atmospheric concentration of carbon dioxide is typically less than 400 ppm by volume. Thus, in some embodiments of the present invention the enriched nanostructure composition is manufactured in the presence of carbon dioxide at a concentration of at least 400 ppm, or at least 600 ppm or at least 800 ppm.

In some embodiments of the present invention the enriched nanostructure composition is manufactured in the presence of gas jet. In some embodiments of the present invention the enriched nanostructure composition is manufactured in the presence of gas, preferably gas jet, at a temperature which is substantially below the ambient temperature.

A solid powder (e.g., a mineral, a ceramic powder, a glass powder, a metal powder, a synthetic polymer, etc.) can be heated to a sufficiently high temperature (for example, about 700° C. or more, e.g., about 850° C.), and subsequently immersed in a cold liquid in the presence of a gas medium. In some embodiments of the present invention the liquid is water below its density anomaly temperature, e.g., 3° C. or 2° C. Substantially contemporaneously with the immersion, the cold liquid, powder and gas medium are irradiated by electromagnetic radiofrequency radiation, e.g., 500 MHz, 750 MHz or more.

The gas phase can be introduced to the composition in more than one way. In some embodiments of the present invention, the heated powder is passed through a gas medium, typically a flow of gas medium, prior to the immersion of the powder in the cold liquid. In some embodiments of the present invention, a gas medium is introduced into the cold liquid prior to or substantially contemporaneously with the immersion. The gas medium is introduced to the liquid in the form of bubbles.

The gas medium is preferably hydrophobic. Representative examples of suitable gas media include, without limitation, carbon dioxide (CO₂), oxygen (O), nitrogen (N), sulfur dioxide (SO₂), hydrogen (H), fluorine (F), methane (CH₄), hexane (C₆H₁₄), hexafluoroethane (C₂F₆) and air. In various exemplary embodiments of the invention the gas medium is carbon dioxide (CO₂).

It has been demonstrated by the present inventor that during the production process described above, some of the large agglomerates of the source powder disintegrate and some of the individual particles of the source powder alter their shape and become spherical nanostructures. It is postulated [Katsir et al., “The Effect of rf-Irradiation on Electrochemical Deposition and its Stabilization by Nanoparticle Doping”, Journal of The Electrochemical Society, 154(4) D249-D259, 2007] that during the production process, nanobubbles are generated by the radiofrequency treatment and cavitation is generated due to the injection of hot particles into water below the anomaly temperature. Since the water is kept below the anomaly temperature, the hot particles cause local heating that in turn leads to a local reduction of the specific volume of the heated location that in turn causes under pressure in other locations. It is postulated that during the process and a time interval of a few hours or less following the process, the water goes through a self-organization process that includes an exchange of gases with the external atmosphere and selective absorption of the surrounding electromagnetic radiation. It is further postulated that the self-organization process leads to the formation of the stable structured distribution composed of the nanobubbles and the nanostructures.

During reduction of the present invention to practice the present inventor has unexpectedly discovered that the above production process effects the generation of a stable or meta-stable gas phase in the liquid. It is hypnotized that in any of the above embodiments for introducing the gas medium into the composition, the radiofrequency treatment results in formation of nanobubbles of the gas medium, and that these nanobubbles are stabilized by the aforementioned self-organization process.

Many types of materials can be used as the solid powder. Representative examples include, without limitation, barium titanate (BaTiO₃), WO₃, Ba₂F₉O₁₂, barium sulfate (BaSO₄). The present inventors unexpectedly found that hydroxyapatite (HA) may also be used in the formulation of the composition. Hydroxyapatite is specifically preferred as it is characterized by intoxicity and is generally FDA approved for human therapy. It will be appreciated that many hydroxyapatite powders are available from a variety of manufacturers such as from Sigma, Aldrich and Clarion Pharmaceuticals (e.g. Catalogue No. 1306-06-5).

The concentration of the nanostructures in the nanostructure composition is not limited. A preferred concentration is below 10²⁰ nanostructures per litter, more preferably below 10¹⁵ nanostructures per litter. One ordinarily skilled in the art would appreciate that with such concentrations, the average distance between the nanostructures in the composition is rather large, of the order of microns. It was demonstrated by the present inventor that the nanostructure composition of the present facilitate long range interactions between the nanostructures. It is postulated that such interaction allows the existence of stable gas phase in the spaces between the nanostructures.

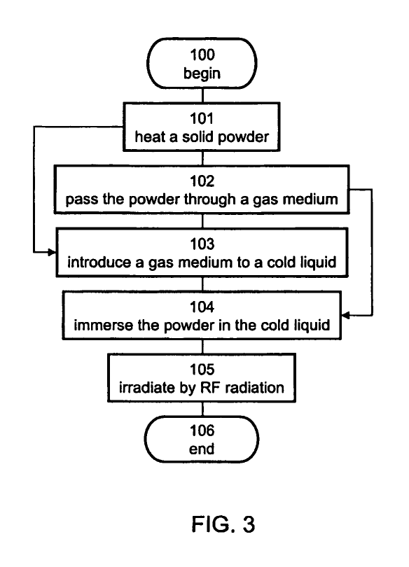

Reference is now made to FIG. 3 which is a flowchart diagram of a method for fabricating an enriched composition, according to various exemplary embodiments of the present invention. The method can be used, for example, for fabricating composition 18 described above.

It is to be understood that, unless otherwise defined, the method phases described hereinbelow can be executed either contemporaneously or sequentially in many combinations or orders of execution. Specifically, the ordering of the flowchart diagram is not to be considered as limiting. For example, two or more method phases, appearing in the following description or in the flowchart diagram in a particular order, can be executed in a different order (e.g., a reverse order) or substantially contemporaneously. Additionally, several method phases described below are optional and may not be executed.

The method begins at 100 and continues to 101 in which a solid powder (e.g., a mineral, a ceramic powder, a glass powder, a metal powder, a synthetic polymer, etc.) can be heated to a sufficiently high temperature (for example, about 700° C. or more, e.g., about 850° C.).

Optionally, the method continuous to 102 at which the heated powder is passed through a gas medium, such as, but not limited to, one of the aforementioned hydrophobic gas media. Preferably, the heated powder is passed through a jet of gas.

Optionally, the method continuous to 103 at which a gas medium, such as, but not limited to, one of the aforementioned hydrophobic gas media, is introduced into a cold liquid. The gas medium can be introduced into the liquid by in the form of a gas jet which penetrates the liquid and generates bubbles therein.

In various exemplary embodiments of the invention at least one of phases 102 and 103 is executed. In some embodiments of the present invention both phases 102 and 103 are executed. In various exemplary embodiments of the invention the gas medium is at a temperature which is below 6° C. or below 5° C., e.g., at about 4° C. or less.

The method continues to 104 at which the powder is immersed in the cold liquid. In embodiments in which phase 102 of the method is executed, the powder carries molecules of the gas medium therewith while being immersed in the cold liquid. In embodiments in which phase 103 is executed, the gas medium can be introduced prior to, subsequently or during phase 104.

In various exemplary embodiments of the invention the liquid is water. In these embodiments, the water is at a temperature which is below its density anomaly temperature, e.g., 3° C. or 2° C.

At 105 the cold liquid, gas medium and powder are irradiated by electromagnetic radiofrequency radiation, e.g., 500 MHz, 750 MHz or more.

The method ends at 106.

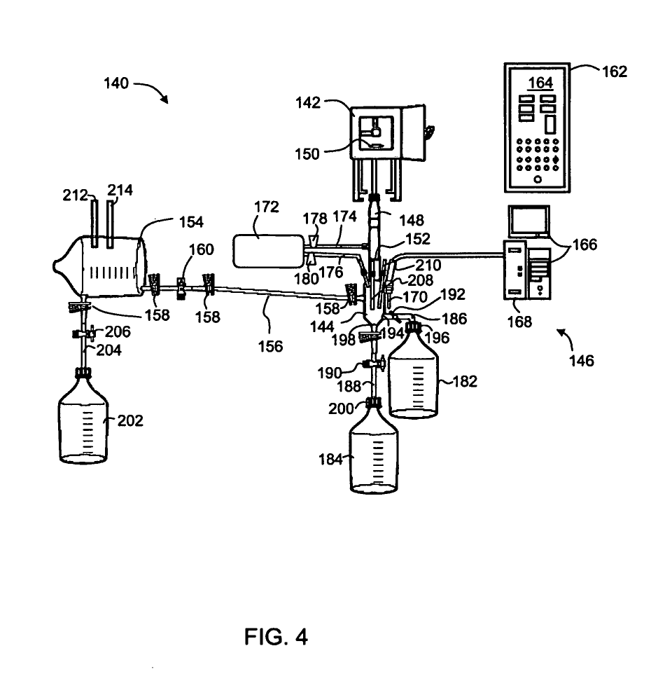

Reference is now made to FIG. 4 which is a schematic illustration of a system 140 for fabricating an enriched composition, according to various exemplary embodiments of the present invention. System 140 can be used, for example, for fabricating composition 18 described above.

System 140 comprises a furnace 142 configured for heating a solid powder (e.g., a mineral, a ceramic powder, a glass powder, a metal powder, a synthetic polymer, etc.) to a sufficiently high temperature (for example, about 700° C. or more, e.g., about 850° C.), a container 144 for holding a liquid (not shown) at a sufficiently low temperature (e.g., water at a temperature which is below its density anomaly temperature), and a radiofrequency unit 146 for generating and transmitting radiofrequency radiation.

In some embodiments of the present invention system 140 comprises a liquid level measuring device 208 for measuring the level of liquid in container 144. In some embodiments of the present invention system 140 comprises a temperature measuring device 210 for measuring the temperature of the liquid in container 144. Device 208 and/or device 210 can communicate with a control unit 162 which can monitor and optionally display the liquid level and/or temperature.

Furnace 142 is connected to container 144 via a projection sleeve 148 configured to receive the heated powder (not shown) at an outlet port 150 of furnace 142 and drop it into container 144 through a powder inlet port 152 formed at the upper part of container 144.

In various exemplary embodiments of the invention container 144 is in fluid communication with a liquid reservoir 154 which supplies the liquid to container 144 via a liquid conduit 156. Conduit 156 can be laid between reservoir 154 and container 144 by means of on or more connectors 158. Liquid flow within conduit 156 can be controlled by a valve 160 mounted on conduit 156. Valve 160 can be operated manually by the user or automatically by a control unit 162, having a user interface 164 through which various production parameters can be selected.

In some embodiments of the present invention system 140 comprises an additional liquid level measuring device 212 for measuring the level of liquid in reservoir 154. In some embodiments of the present invention system 140 comprises an additional temperature measuring device 214 for measuring the temperature of the reservoir 154. Device 212 and/or device 214 can communicate with control unit 162 which can monitor and optionally display the liquid level and/or temperature.

Radiofrequency unit 146 typically comprises a radiofrequency control unit 166 (e.g., a data processor), a radiofrequency generator and transmitter 168 and a radiofrequency antenna device 170. Unit 166 controls generator and transmitter 168 which generates radiofrequency radiation and transmits via antenna device 170 such that at least part of the radiofrequency radiation enters container 144. Device 170 can be positioned within container 144 or adjacent thereto. In the latter case, container 144 is made of a material which allows transmission of radiofrequency radiation therethrough.

System 140 further comprises a gas reservoir 172 which is configured to supply gas medium to at least one of: projection sleeve 148 and liquid container 144, via gas conduits 174 and 176, respectively. Gas flow through conduits 174 and 176 can be controlled by gas valves 178 and 180, respectively, each of which can be independently operated manually by the user or automatically by control unit 162.

In various exemplary embodiments of the invention system 140 comprises at least one of a product container 182 and a waste container 184, which is/are can be in fluid communication with liquid container 144. Product container 182 can be connected to liquid container 144 via a product conduit 186 which is configured for receiving the product (e.g., composition 18) at a product outlet port 194 of container 144 and introduce it through a product inlet port 196 at product container 184. Waste container 184 can be connected to liquid container 144 via a waste conduit 188 which is configured for receiving the waste (e.g., excess liquid, powder debris) at a waste outlet port 198 of container 144 and introduce it through a waste inlet port 200 at waste container 184.

Waste flow through waste conduit 188 can be controlled by a waste valve 190 and product flow through product conduit 186 can be controlled by a product valve 192. Each of valves 190 and 192 can be independently operated manually by the user or automatically by control unit 162.

An additional waste container 202 can be connected to reservoir 154 via a conduit 204 for draining untreated liquid from reservoir 154. Conduit 204 can be connected to reservoir 154 via a connector 158 and liquid flow through conduit 204 can be controlled via a valve 206, which can be operated manually by the user or automatically by control unit 162.

In operation, the solid powder is introduced into furnace 142 and heated to a sufficiently high temperature (for example, about 700° C. or more, e.g., about 850° C.), and the liquid in reservoir 154 is cooled to a sufficiently low temperature (e.g., 2-3° C. in the case of water). Valve 160 is opened to allow the cold liquid to flow to container 144. Valve 178 and/or 180 is opened and the gas medium from gas reservoir 172 is introduced to sleeve 148 and/or container 144. Unit 166 activates radiofrequency generator and transmitter 168 and container 144 is irradiated by radiofrequency radiation emitted from antenna device 170. Sleeve 148 drops the heated powder into container 144.

Interactions between the nanostructures of the enriched nanostructure composition of the present embodiments (both long range and short range interactions) facilitate self organization capability of the enriched nanostructure composition, similar to a self organization of bacterial colonies. When a bacterial colony grows, self-organization allows it to cope with adverse external conditions and to “collectively learn” from the environment for improving the growth rate. Similarly, the long range interaction and thereby the long range order of the enriched nanostructure composition allows the enriched nanostructure composition to perform self-organization, so as to adjust to different environmental conditions, such as, but not limited to, different temperatures, electrical currents, radiation and the like.

The long range order of the enriched nanostructure composition of the present embodiments is best seen when the enriched nanostructure composition is subjected to an electrochemical deposition (ECD) experiment (see also the Examples section that follows).

ECD is a process in which a substance is subjected to a potential difference (for example using two electrodes), so that an electrochemical process is initiated. A particular property of the ECD process is the material distribution obtained thereby. During the electrochemical process, the potential measured between the electrodes at a given current, is the sum of several types of over-voltage and the Ohmic drop in the substrate. The size of the Ohmic drop depends on the conductivity of the substrate and the distance between the electrodes. The current density of a specific local area of an electrode is a function of the distance to the opposite electrode. This effect is called the primary current distribution, and depends on the geometry of the electrodes and the conductivity of the substrate.

When the potential difference between the electrodes is large, compared to the equilibrium voltage, the substrates experience a transition to a non-equilibrium state, and as a result, structures of different morphologies are formed. It has been found [E. Ben-Jacob, “From snowflake formation to growth of bacterial colonies,” Cont. Phys., 1993, 34(5)] that systems in non-equilibrium states may select a morphology and/or experience transitions between two morphologies: dense branching morphology and a dendritic morphology.

According to some embodiments of the present invention when the enriched nanostructure composition of the present embodiments is placed in an electrochemical deposition cell, a predetermined morphology (e.g., dense branching and/or dendritic) is formed. Preferably, the enriched nanostructure composition of the present embodiments is capable of preserving an electrochemical signature on the surface of the cell even when replaced by a different liquid (e.g., water). More specifically, According to some embodiments of the present invention, when the enriched nanostructure composition is first contacted with the surface of the electrochemical deposition cell and then washed by a predetermined wash protocol, an electrochemical signature of the composition is preserved on the surface of the cell.

The long range interaction of the nanostructures can also be demonstrated by subjecting the enriched nanostructure composition of the present embodiments to new environmental conditions (e.g., temperature change) and investigating the effect of the new environmental conditions on one or more physical quantities which are related to the interaction between the nanostructures in the composition. One such physical quantity is ultrasonic velocity. In some embodiments of the present invention the enriched nanostructure composition is characterized by an enhanced ultrasonic velocity relative to water.

An additional characteristic of the present invention is a small contact angle between the enriched nanostructure composition and solid surface. Preferably, the contact angle between the enriched nanostructure composition and the surface is smaller than a contact angle between liquid (without the nanostructures) and the surface. One ordinarily skilled in the art would appreciate that small contact angle allows the enriched nanostructure composition to “wet” the surface in larger extent. It is to be understood that this feature of the present invention is not limited to large concentrations of the nanostructures in the liquid, but rather also to low concentrations, with the aid of the above-mentioned long range interactions between the nanostructures.

In some embodiments of the present invention the enriched nanostructure composition of the present embodiments is characterized by a non-vanishing circular dichroism signal. Circular dichroism is an optical phenomenon that results when a substance interacts with plane polarized light at a specific wavelength. Circular dichroism occurs when the interaction characteristics of one polarized-light component with the substance differ from the interaction characteristics of another polarized-light component with the substance. For example, an absorption band can be either negative or positive depending on the differential absorption of the right and left circularly polarized components for the substance.

It is recognized that non-vanishing circular dichroism signal of the enriched nanostructure composition indicates that the enriched nanostructure composition is an optically active medium. Thus, the enriched nanostructure composition of the present embodiments can alter the polarization of light while interacting therewith. The present inventor postulates that the optical activity of the enriched nanostructure composition of the present embodiments is a result of the long-range order which is manifested by the aforementioned formation of stable structured distribution of nanobubbles and nanostructures.

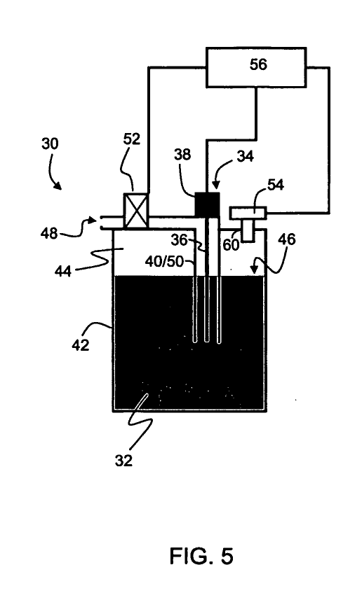

Reference is now made to FIG. 5 which is a schematic illustration of an apparatus 30 for recycling a gas, according to various exemplary embodiments of the present invention. In various exemplary embodiments of the invention apparatus 30 comprises a composition 32, and an excitation device 34 for exciting composition 32. Composition 32 is typically contained in a chamber 42 which may optionally serve as a body of apparatus 30 or part thereof.

Composition 32 is preferably capable of releasing gas when excitation device 34 is active and collecting the gas upon deactivation of excitation device 34. Composition 32 can comprise liquid, nanostructure and a gas phase. In various exemplary embodiments of the invention the gas in composition 32 is hydrophobic. Representative examples of suitable gas media include, without limitation, carbon dioxide (CO₂), oxygen (O), nitrogen (N), sulfur dioxide (SO₂), hydrogen (H), fluorine (F), methane (CH₄), hexane (C₆H₁₄), hexafluoroethane (C₂F₆) and air. In various exemplary embodiments of the invention the gas medium is carbon dioxide (CO₂).

Composition 32 can be similar to composition 18.

Excitation device 34 is configured in accordance with the composition being employed. It was found by the present inventor that the composition can release the gas in response to irradiation with radiofrequency radiation. Thus, according to an embodiment of the invention, device 34 irradiates composition 32 with radiofrequency radiation such as to excite the composition to an excited state. As long as the radiofrequency radiation is applied, composition 32 remains in its excited state and the gas is released therefrom (see FIG. 2). Upon deactivation of device 34, composition 32 experiences a transition to its stable state and, during the transition, gas is collected by the composition. In this embodiment, device 34 comprises a radiofrequency transmitter. Typically, device 34 comprises a radiofrequency antenna 36 and a radiofrequency generator 38, as known in the art.

When the composition is manufactured by a “top-down” process as described above, the frequency of the radiation can be the same as the frequency employed during the production process. Yet, it is not intended to limit the scope of the present invention to any specific frequency.

While the embodiments above were described with a particular emphasis to excitation using radiofrequency radiation, it is to be understood that more detailed reference to radiofrequency radiation is not to be interpreted as limiting the scope of the invention in any way. Excitation device 34 can be any device capable of exciting composition 32. Representative examples include, without limitation, excitation by heat, excitation by longitudinal waves (e.g., ultrasonic excitation) and excitation by shock waves.

Apparatus 30 optionally comprises a gas separating mechanism 40 designed and constructed to separate gases 44 contacting composition 32 from the gas (e.g., CO₂ gas) released from composition 32. As shown in FIG. 5, gasses 44 are typically confined within chamber 42 and contact a surface 46 of composition 32. Mechanism 40 can be a sleeve 50 extending from the environment into composition 32. In this embodiment, antenna 36 is disposed within the sleeve, and a fluid communication can be established between free gas molecules (e.g., CO₂ molecules) accumulating near the antenna and an outlet 48 of the sleeve. Additionally, gases contacting the composition are substantially prevented from entering the sleeve. An outlet valve 52 is optionally positioned in sleeve 50 so as to control release of the gas to the environment. Outlet valve 52 can be mechanical or electrical as desired.

Optionally and preferably, apparatus 30 further comprises an inlet valve 54 positioned at an inlet 60 in the wall of chamber 42 so as to allow controlling fluid communication between the environment and surface 46 of composition 32. Apparatus 30 may further comprise a control unit 56 which control the operation of apparatus 30. For example, control unit 56 can be configured for activating and deactivating excitation device 34, for opening and closing outlet valve 52 and/or for opening and closing inlet valve 54.

In operation, excitation device 34 is activated to excite composition 32, outlet valve 52 is preferably open and inlet valve 54 is preferably close. While composition 32 is excited, an efflux of gas (e.g., CO₂ gas) exits through outlet 48. During the period of gas release, the local concentration of gas near apparatus 30, particularly near outlet 48 is relatively high. For example, when the gas is CO₂, a local CO₂ concentration of the order of 1000 ppm by volume is accumulated near outlet 48. Bursts of several thousands ppm by volume to a few tens of thousands ppm by volume are also contemplated.

After a certain period of time in which the gas is released, device 34 is deactivated, and inlet valve 54 is brought to its open position. Due to the recycling property of composition 32, an influx of the gas (e.g., environmental CO₂) enters through inlet 60 into chamber 42 and composition 32 experiences a transition from its excited state to a less energetic state.

In various exemplary embodiments of the invention control unit 56 intermittently activates excitation device 34, according to a predetermined scenario. The operation scenarios can be expressed as active/inactive ratios. Typically, but not obligatorily, the operation of excitation device 34 is according to an active/inactive ratio which is from about 1/20 to about 2/1. In some embodiments of the present invention the operation of excitation device 34 is according to an active/inactive ratio of about 1/10. In this embodiment device 34 is active about one tenth and inactive about nine tenths of the overall operation time of apparatus 30.

Control unit 56 can also control outlet valve 54 while excitation device is active. When outlet valve 54 is closed and device 34 is active, the concentration of gas within sleeve 50 is increased. This can ultimately results in bursts of high gas concentrations upon opening of valve 54. In various exemplary embodiments of the invention control unit 56 controls outlet valve 54 according to a predetermined scenario which can be expressed in terms of a closed/open ratio. For example, valve 54 can operate according to a close/open ratio of 30/1 whereby the valve is closed for about 30 seconds and opened for about 1 second. Many other operation scenarios can be employed. In the Examples section that follows, it is demonstrated that a sufficiently high concentration of CO₂ can be achieved for many operation scenarios.

Apparatus 30 can serve for attracting insects, such as, but not limited to, female mosquitoes or other biting flies (Diptera). This embodiment is particularly useful when the gas phase of composition 32 is CO₂. Female mosquitoes can smell the CO₂ that a host (a source of blood) exhales at about 30 meters away from the host. Because CO₂ is present in the atmosphere, female mosquitoes respond to higher-than-normal concentrations. The ability of apparatus 30 to generate high local concentrations of CO2, mimics the breathing behavior of animals hence attracts the insects.

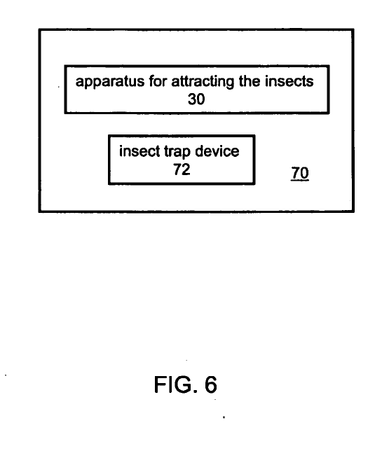

Reference is now made to FIG. 6 which is a schematic illustration of an apparatus 70 for trapping insects, according to various exemplary embodiments of the present invention. Apparatus 70 preferably comprises an apparatus for attracting the insects, e.g., apparatus 30, and an insect trap device 72 designed and configured to trap insects which are nearby the outlet of apparatus 30.

The trap device can be any trap device known in the art. For example, the trap device can include fan impeller configured to suck the insects into a cone-shaped net secured under the fan impeller for killing the mosquitoes, which spirally impact against the net. Optionally, The trap device includes a sticky body having a sticky surface to trap then insects on at least an external surface of the sticky body.

It is expected that during the life of this patent many relevant incest traps will be developed and the scope of the term an insect trap device is intended to include all such new technologies a priori.

Apparatus 30 can also be used for enhancing plant growth. In this embodiment, the gas phase of composition 32 is preferably CO₂. CO₂ is preferably collected during dark hours and released during daylight hours when photosynthesis occurs. During daylight hours, the CO₂ can be transported to a tract of plants from which it can be distributed to the plants in the tract. The distribution can be in direct manner (e.g., location apparatus 30 near the plants) or using a distribution system, such as, but not limited to, an existing localized irrigation systems in which case the CO₂ can be distributed under field conditions.

Ideally, the CO₂ is delivered to the plants when the temperature is between 20° C. and 27° C., which is the optimal temperature range for photosynthesis. It will be appreciated that CO₂ may be delivered at other temperatures. The advantage of the present embodiment is that it enhances plant growth and reduces the amount of fertilizer required to grow the plants.

In some embodiments of the present invention the enriched nanostructure composition is characterized by an enhanced ability to dissolve or disperse a substance as compared to water.

As used herein, the term “dissolve” refers to the ability of the enriched nanostructure composition of the present embodiments to make soluble or more soluble in an aqueous environment.

As used herein, the term “disperse” relates to the operation of putting into suspension according to the degree of solubility of the substance.

Thus, according to an aspect of some embodiments of the present invention, there is provided a method of dissolving or dispersing a substance comprising contacting the substance with nanostructures and liquid under conditions which allow dispersion or dissolving of the substance.

The nanostructures and liquid of the present invention may be used to dissolve/disperse any substance (e.g. active agent) such as a protein, a nucleic acid, a small molecule and a carbohydrate, including pharmaceutical agents such as therapeutic agents, cosmetic agents and diagnostic agents.

A therapeutic agent can be any biological active factor such as, for example, a drug, a nucleic acid construct, a vaccine, a hormone, an enzyme, small molecules such as for example iodine or an antibody. Examples of therapeutic agents include, but are not limited to, antibiotic agents, free radical generating agents, anti fungal agents, anti-viral agents, non-nucleoside reverse transcriptase inhibitors, protease inhibitors, non-steroidal anti inflammatory drugs, immunosuppressants, anti-histamine agents, retinoid agents, tar agents, antipruritic agents, hormones, psoralen, and scabicide agents. Nucleic acid constructs deliverable by the present invention can encode polypeptides (such as enzymes ligands or peptide drugs), antisense RNA, or ribozymes.

A cosmetic agent of the present invention can be, for example, an anti-wrinkling agent, an anti-acne agent, a vitamin, a skin peel agent, a hair follicle stimulating agent or a hair follicle suppressing agent. Examples of cosmetic agents include, but are not limited to, retinoic acid and its derivatives, salicylic acid and derivatives thereof, sulfur-containing D and L amino acids and their derivatives and salts, particularly the N-acetyl derivatives, alpha-hydroxy acids, e.g., glycolic acid, and lactic acid, phytic acid, lipoic acid and many other agents which are known in the art.

A diagnostic agent of the present invention may be an antibody, a chemical or a dye specific for a molecule indicative of a disease state.

Other therapeutic, cosmetic agent and diagnostic agent are described hereinunder.

The substance may be dissolved in a solvent prior or following addition of the enriched nanostructure composition of the present embodiments in order to aid in the solubilizing process. It will be appreciated that the present invention contemplates the use of any solvent including polar, non-polar, organic, (such as ethanol or acetone) or non-organic to further increase the solubility of the substance.

The solvent may be removed (completely or partially) at any time during the solubilizing process so that the substance remains dissolved/dispersed in the enriched nanostructure composition of the present embodiments. Methods of removing solvents are known in the art such as evaporation (i.e. by heating or applying pressure) or any other method.

A further characteristic of the enriched nanostructure composition of the present embodiments is buffering capacity. In some embodiments of the present invention the enriched nanostructure composition is characterized by an enhanced buffering capacity as compared to water.

As used herein, the phrase “buffering capacity” refers to the composition's ability to maintain a stable pH stable as acids or bases are added.

Yet a further characteristic of the enriched nanostructure composition of the present embodiments is protein stability. In some embodiments of the present invention the enriched nanostructure composition is characterized by an enhanced ability to stabilize proteins. Thus, for example, the enriched nanostructure composition can shield and stabilize proteins from the effects of heat

In some embodiments of the present invention the enriched nanostructure composition is capable of facilitating the increment of bacterial colony expansion rate and phage-bacteria or virus-cell interaction, even when the solid powder used for preparing the enriched nanostructure composition is toxic to the bacteria. The unique process by which the enriched nanostructure composition is produced, which, as stated, allows the formation of the envelope surrounding core material, significantly suppresses any toxic influence of the enriched nanostructure composition on the bacteria or phages.

An additional characteristic of the enriched nanostructure composition of the present embodiments is related to the so called zeta (ζ) potential. ζ potential is related to physical phenomena called electrophoresis and dielectrophoresis in which particles can move in a liquid under the influence of electric fields present therein. The ζ potential is the electric potential at a shear plane, defined at the boundary between two regions of the liquid having different behaviors. The electrophoretic mobility of particles (the ratio of the velocity of particles to the field strength) is proportional to the ζ potential.

Being a surface related quantity, the ζ potential is particularly important in systems with small particle size, where the total surface area of the particles is large relative to their total volume, so that surface related phenomena determine their behavior.

According to some embodiments of the present invention, the enriched nanostructure composition is characterized by a ζ potential which is substantially larger than the ζ potential of the liquid per se. Large ζ potential corresponds to enhanced mobility of the nanostructures in the liquid, hence, it may contribute, for example, to the formation of special morphologies in the electrochemical deposition process.

There are many methods of measuring the ζ potential of the enriched nanostructure composition, including, without limitation, microelectrophoresis, light scattering, light diffraction, acoustics, electroacoustics etc. For example, one method of measuring ζ potential is disclosed in U.S. Pat. No. 6,449,563, the contents of which are hereby incorporated by reference.

The present embodiments also relate to the field of molecular biology research and diagnosis, particularly to nucleic acid amplification techniques, such as, but not limited to, polymerase chain reaction (PCR), ligase chain reaction (LCR), strand displacement amplification (SDA) and self-sustained sequence replication (SSSR).

In some embodiments of the present invention the enriched nanostructure composition of the present embodiments is capable of improving the efficiency of a nucleic acid amplification process. As used herein, the phrase “improving the efficiency of a nucleic acid amplification process” refers to enhancing the catalytic activity of a DNA polymerase in PCR procedures, increasing the stability of the proteins required for the reaction, increasing the sensitivity and/or reliability of the amplification process and/or reducing the reaction volume of the amplification reaction. In some embodiments of the present invention the enhancement of catalytic activity is preferably achieved without the use of additional cofactors such as, but not limited to, magnesium or manganese. As will be appreciated by one of ordinary skill in the art, the ability to employ a magnesium-free or manganese-free PCR is highly advantageous. This is because the efficiency of a PCR procedure is known to be very sensitive to the concentration of the cofactors present in the reaction. An expert scientist is often required to calculate in advance the concentration of cofactors or to perform many tests, with varying concentrations of cofactors, before achieving the desired amplification efficiency.

The use of the enriched nanostructure composition of the present embodiments thus allows the user to execute a simple and highly efficient multi-cycle PCR procedure without having to calculate or vary the concentration of cofactors in the PCR mix.

In some embodiments of the present invention the polymerase chain reaction takes place devoid of any additional buffers or liquids. One of the major problems associated with the application of PCR to clinical diagnostics is the susceptibility of PCR to carryover contamination. These are false positives due to the contamination of the sample with molecules amplified in a previous PCR. The use of the enriched nanostructure composition of the present embodiments as a sole PCR mix significantly reduces the probability of carryover contamination, because the entire procedure can be carried out without the need for any additional buffers or liquids, hence avoiding the risk of contamination.

In some embodiments of the present invention the enriched nanostructure composition of the present embodiments enhances the sensitivity and decrease the reaction volume of a real-time PCR reaction. As used herein a real-time PCR reaction refers to a PCR reaction which is carried out in the presence of a double stranded DNA detecting molecule (e.g., dye) during each PCR cycle.

Furthermore, the enriched nanostructure composition of the present embodiments may be used in very small volume PCR reactions (e.g. 2 μls). In some embodiments of the present invention the enriched nanostructure composition is used in heat dehydrated multiplex PCR reactions.

Thus, according to some embodiments of the present invention there is provided a kit for polymerase chain reaction. The PCR kit of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

In some embodiments of the present invention the kit comprises, preferably in separate packaging, a thermostable DNA polymerase, such as, but not limited to, Taq polymerase and the enriched nanostructure composition of the present embodiments.

In some embodiments of the present invention the kit is used for real-time PCR kit and additionally comprises at least one real-time PCR reagent such as a double stranded DNA detecting molecule. The components of the kit may be packaged separately or in any combination.

As used herein the phrase “double stranded DNA detecting molecule” refers to a double stranded DNA interacting molecule that produces a quantifiable signal (e.g., fluorescent signal). For example such a double stranded DNA detecting molecule can be a fluorescent dye that (1) interacts with a fragment of DNA or an amplicon and (2) emits at a different wavelength in the presence of an amplicon in duplex formation than in the presence of the amplicon in separation. A double stranded DNA detecting molecule can be a double stranded DNA intercalating detecting molecule or a primer-based double stranded DNA detecting molecule.

A double stranded DNA intercalating detecting molecule is not covalently linked to a primer, an amplicon or a nucleic acid template. The detecting molecule increases its emission in the presence of double stranded DNA and decreases its emission when duplex DNA unwinds. Examples include, but are not limited to, ethidium bromide, YO-PRO-1, Hoechst 33258, SYBR Gold, and SYBR Green I. Ethidium bromide is a fluorescent chemical that intercalates between base pairs in a double stranded DNA fragment and is commonly used to detect DNA following gel electrophoresis. When excited by ultraviolet light between 254 nm and 366 nm, it emits fluorescent light at 590 nm. The DNA-ethidium bromide complex produces about 50 times more fluorescence than ethidium bromide in the presence of single stranded DNA. SYBR Green I is excited at 497 nm and emits at 520 nm. The fluorescence intensity of SYBR Green I increases over 100 fold upon binding to double stranded DNA against single stranded DNA. An alternative to SYBR Green I is SYBR Gold introduced by Molecular Probes Inc. Similar to SYBR Green I, the fluorescence emission of SYBR Gold enhances in the presence of DNA in duplex and decreases when double stranded DNA unwinds. However, SYBR Gold's excitation peak is at 495 nm and the emission peak is at 537 nm. SYBR Gold reportedly appears more stable than SYBR Green I. Hoechst 33258 is a known bisbenzimide double stranded DNA detecting molecule that binds to the AT rich regions of DNA in duplex. Hoechst 33258 excites at 350 nm and emits at 450 nm. YO-PRO-1, exciting at 450 nm and emitting at 550 nm, has been reported to be a double stranded DNA specific detecting molecule. In a preferred embodiment of the present invention, the double stranded DNA detecting molecule is SYBR Green I.

A primer-based double stranded DNA detecting molecule is covalently linked to a primer and either increases or decreases fluorescence emission when amplicons form a duplex structure. Increased fluorescence emission is observed when a primer-based double stranded DNA detecting molecule is attached close to the 3′ end of a primer and the primer terminal base is either dG or dC. The detecting molecule is quenched in the proximity of terminal dC-dG and dG-dC base pairs and dequenched as a result of duplex formation of the amplicon when the detecting molecule is located internally at least 6 nucleotides away from the ends of the primer. The dequenching results in a substantial increase in fluorescence emission. Examples of these type of detecting molecules include but are not limited to fluorescein (exciting at 488 nm and emitting at 530 nm), FAM (exciting at 494 nm and emitting at 518 nm), JOE (exciting at 527 and emitting at 548), HEX (exciting at 535 nm and emitting at 556 nm), TET (exciting at 521 nm and emitting at 536 nm), Alexa Fluor 594 (exciting at 590 nm and emitting at 615 nm), ROX (exciting at 575 nm and emitting at 602 nm), and TAMRA (exciting at 555 nm and emitting at 580 nm). In contrast, some primer-based double stranded DNA detecting molecules decrease their emission in the presence of double stranded DNA against single stranded DNA. Examples include, but are not limited to, rhodamine, and BODIPY-FI (exciting at 504 nm and emitting at 513 nm). These detecting molecules are usually covalently conjugated to a primer at the 5′ terminal dC or dG and emit less fluorescence when amplicons are in duplex. It is believed that the decrease of fluorescence upon the formation of duplex is due to the quenching of guanosine in the complementary strand in close proximity to the detecting molecule or the quenching of the terminal dC-dG base pairs.

Additionally, the PCR and real-time PCR kits may comprise at least one dNTP, such as, but not limited to, dATP, dCTP, dGTP, dTTP. Analogues such as dITP and 7-deaza-dGTP are also contemplated.

According to some embodiments of the present invention the kits may further comprise at least one control template DNA and/or at least one at least one control primer to allow the user to perform at least one control test to ensure the PCR performance.

According to an aspect of some embodiments of the present invention there is provided a method of amplifying a DNA sequence. In a first step of the method, the enriched nanostructure composition of the present embodiments is provided, and in a second step, a plurality of PCR cycles is executed on the DNA sequence in the presence of the enriched nanostructure composition.

The PCR cycles can be performed in any way known in the art, such as, but not limited to, the PCR cycles disclosed in U.S. Pat. Nos. 4,683,195, 4,683,202, 4,800,159, 4,965,188, 5,512,462, 6,007,231, 6,150,094, 6,214,557, 6,231,812, 6,391,559, 6,740,510 and International Patent application No. WO99/11823.

Preferably, in each PCR cycle, the DNA sequence is first treated to form single-stranded complementary strands. Subsequently, pair of oligonucleotide primers which are specific to the DNA sequence are added to the enriched nanostructure composition. The primer pair is then annealed to the complementary sequences on the single-stranded complementary strands. Under proper conditions, the annealed primers extend to synthesize extension products which are respectively complementary to each of the single-strands.

Anchoring polynucleotide to a solid support such as glass beads can be of utmost benefit in the field of molecular biology research and medicine.

As used herein “polynucleotides” are defined as DNA or RNA molecules linked to form a chain of any size.

Polynucleotides may be manipulated in many ways during the course of research and medical applications, including, but not limited to amplification, transcription, reverse transcription, ligation, restriction digestion, transfection and transformation.

As used herein, “ligation” is defined as the joining of the 3′ end of one nucleic acid strand with the 5′ end of another, forming a continuous strand. “Transcription” is defined as the synthesis of messenger RNA from DNA. “Reverse transcription” is defined as the synthesis of DNA from RNA. “Restriction digestion” is defined as the process of cutting DNA molecules into smaller pieces with special enzymes called Restriction Endonucleases. “Transformation” is the process by which bacterial cells take up naked DNA molecules “Transfection” is the process by which cells take up DNA molecules.

Typically, DNA manipulations comprise a sequence of reactions, one following the other. Thus, as a typical example DNA can be initially restriction digested, amplified and then transformed into bacteria. Each reaction is preferably performed under its own suitable reaction conditions requiring its own specific buffer. Typically, in between each reaction, the DNA or RNA sample must be precipitated and then reconstituted in its new appropriate buffer. Repeated precipitations and reconstitutions takes time and more importantly leads to loss of starting material, which can be of utmost relevance when this material is rare. By anchoring the polynucleotides to a solid support, this is avoided.

Thus, according to an aspect of some embodiments of the present invention, the enriched nanostructure composition of the present embodiments is capable of allowing the manipulation of at least one macromolecule in the presence of a solid support, whereby each of the nanostructures comprise a core material of a nanometric size surrounded by an envelope of ordered fluid molecules, the core material and the envelope of ordered fluid molecules being in a steady physical state.

The solid support can be any solid support capable of binding DNA and RNA while allowing access of other molecules to bind and interact with the DNA and RNA in subsequent reactions as discussed above.

In some embodiments of the present invention glass beads, which are capable of anchoring polynucleotides, require the enriched nanostructure composition in order for the polynucleotides to remain intact. In some embodiments of the present invention DNA undergoing PCR amplification in the presence of glass beads requires the presence of the enriched nanostructure composition for the PCR product to be visualized.

Beside nucleic acid amplification, the enriched nanostructure composition of the present embodiments can be used as a buffer or an add-on to an existing buffer, for improving many chemical and biological assays and reactions.

Hence, in one embodiment the enriched nanostructure composition of the present embodiments can be used to at least partially de-fold DNA molecules.

In another embodiment, the enriched nanostructure composition of the present embodiments can be used to facilitate isolation and purification of DNA.

In yet another embodiment, the enriched nanostructure composition of the present embodiments can be used to enhance nucleic acid hybridization. The nucleic acid may be a DNA and/or RNA molecule (i.e., nucleic acid sequence or a single base thereof).

One of the nucleic acids may be bound to a solid support (e.g. a DNA chip). Examples of DNA chips include but are not limited to focus array chips, Affymetrix chips and Illumina bead array chips.

Since the enriched nanostructure composition was shown to enhance hybridization, the present invention may be particularly useful in detecting genes which have low expression levels.

In an additional embodiment, the enriched nanostructure composition of the present embodiments can be used for 6stabilizing enzyme activity of many enzymes, either bound or unbound enzymes, such as, but not limited to, Alkaline Phosphatase or β-Galactosidase.

In still another embodiment, the enriched nanostructure composition of the present embodiments can also be used for enhancing binding of macromolecule to a solid phase matrix. In some embodiments of the present invention the enriched nanostructure composition enhances binding to both hydrophilic and hydrophobic substances. In addition, the enriched nanostructure composition of the present embodiments can enhance binding to substances having hydrophobic regions and hydrophilic regions. The binding of many macromolecules to the above substances can be enhanced, including, without limitation macromolecule having one or more carbohydrate hydrophilic or carbohydrate hydrophobic regions, antibodies, polyclonal antibodies, lectin, DNA molecules, RNA molecules and the like.

In some embodiments of the present invention the enriched nanostructure composition can be used for increasing a capacity of a column, binding of nucleic acids to a resin and improving gel electrophoresis separation.

The present embodiments also comprise a novel antiseptic composition and methods of using same. Specifically, the present embodiments can be used to sterilize a body surface (e.g., the mouth, as a mouthwash) or an object.

Antiseptics may be employed for a myriad of purposes including application prior to surgical interventions, prior to injections, punctures and prior to inspections of hollow organs when the skin or the mucous membrane has to be disinfected. In addition, antiseptics are also employed for wound treatment and for the therapy of local superficial skin infections (e.g. in fungal infections). Solutions containing antiseptics may be used for caries prophylaxis in the form of mouthwashes.

Mouthwashes are useful for killing bacteria in the oral cavity that are responsible for plaque, gingivitis and bad breathe. In the majority of mouthwashes, ethanol is used as the solvent. Alcohol-containing mouthwashes are disadvantageous as they may cause burning or stinging effects in the mouth of the user, and additionally are thought to predispose the mouth to cancer. Furthermore, alcohol-containing mouthwashes may be problematic for some users including those who cannot, or should not use alcohol because of physiological (e.g. patients undergoing chemotherapy), psychological, social or job related reasons. Therefore, it is highly desired to have novel antiseptic compositions that are devoid of the above limitations.

In some embodiments of the present invention the enriched nanostructure composition of the present embodiments is used for disinfecting a body surface or an object either per se or when used as carriers for antiseptic agents.

In some embodiments of the present invention the enriched nanostructure composition of the present embodiments is effective as a solvent for mouthwash active ingredients (e.g. thymol, methyl salicylate, menthol and eucalyptol). Antiseptic active agents create finer micelles over time, with more dispersion in the enriched nanostructure composition of the present embodiments compared with reverse osmosis (RO). Since the efficacy and taste of antiseptic mouthwashes, is due to the availability or dissolution of their active ingredients (e.g. thymol, methyl salicylate, menthol and eucalyptol), enriched nanostructure compositions of the present embodiments may be an effective solvent for the active ingredients contained in mouthwashes. Furthermore, compositions of the present embodiments may be alcohol free since no additional alcohol was required for dispersion. The compositions of the present embodiments may therefore be used as a replacement of alcohol as a solvent.

In some embodiments of the present invention there is provided an antiseptic composition comprising enriched nanostructure composition and at least one antiseptic agent.

As used herein, the phrase “antiseptic composition” refers to a solid, semi solid or liquid composition which is cytostatic and/or cytotoxic to pathogens such as bacteria, fungi, amoebas, protozoas and/or viruses. Preferably, the antiseptic composition of the present embodiments does not comprise more than 20% alcohol w/v and even more preferably is devoid of alcohol (for the reasons described hereinabove).

Preferably, the enriched nanostructure composition does not cause significant irritation when applied to a body surface of an organism and does not abrogate the biological activity and properties of the dissolved antiseptic agent.

In some embodiments of the present invention the enriched nanostructure composition is able to dissolve or disperse agents in general and antiseptic agents present in strips in particular to a greater extent than water.

In some embodiments of the present invention the enriched nanostructure composition enhances penetration of the antiseptic agent through hydrophobic membranes. The enriched nanostructure composition may also enhance the antiseptic properties of an agent by providing a stabilizing environment (e.g., stabilizing proteins from the effects of heat).

In some embodiments of the present invention the antiseptic properties of the enriched nanostructure composition are expressed or elevated when the composition contacts specific materials, in particular specific biological materials which are typically present in the upper pharynx, (e.g., eukaryotic fungi, protists, methanogenic Archaea or bacteria). On the other hand, no antiseptic properties were observed without presence of such materials. Thus, the enriched nanostructure composition of the present embodiments has dormant antiseptic properties, in the sense that specific biological materials serve as “primers” to the antiseptic process.

In some embodiments of the present invention the antiseptic composition of the present embodiments comprises at least one antiseptic agent.

As used herein the phrase “antiseptic agent” refers to an agent which is cytostatic and/or cytotoxic to pathogens such as bacteria, fungi, amoebas, protozoas and/or viruses.

The antiseptic agent of the antiseptic compositions of the present embodiments is selected according to the intended use of the antiseptic compositions of the present embodiments.

Preferably, the antiseptic agent is stable over a reasonably long shelf-life (e.g. two years), and preferably it should preferably possess substantivity, i.e. a prolonged contact time between the agent and the microbes on which the agent is to induce its effect.

Thus for example, when the antiseptic composition is used for animate administration, the antiseptic agent is preferably a non-toxic antiseptic agent. For example, when used as a mouthwash, the antiseptic agent of the present embodiments is preferably an orally non-toxic antiseptic agent.

As used herein, the phrase “an orally non-toxic antiseptic agent” refers to an antiseptic agent, which is safe (i.e. does not cause unwanted side-effects) at its recommended dose, and when it is administered as directed. For example, if used in a mouthwash, an orally non-toxic antiseptic agent should be non-toxic when rinsed in the mouth, even if a fraction of the antiseptic agent is swallowed whilst rinsing. Oral antiseptic compositions of the present embodiments can be used for the treatment and/or prevention of oral diseases such as dental caries, gingivitis, dental infection, abscess and periodontal diseases.

Examples of orally non-toxic antiseptic agents include, but are not limited to thymol, methyl salicylate, menthol, sodium chloride, hydrogen peroxide, chlorhexidine gluconate, chlorobutanol hemihydrate, phenol and eucalyptol.

Other antiseptic agents which may be used by the present embodiments include, but are not limited to, a monohydric alcohol, a metal compound, a quaternary ammonium compound, iodine, an iodophor or a phenolic compound.

Examples of monohydric alcohols which may be used according to the present embodiments include, but are not limited to ethanol and isopropanol.

Examples of metal compounds which may be used according to the present embodiments include, but are not limited to silver nitrate and silver sulfadiazine.

Examples of quaternary ammonium compound which may be used according to the present embodiments include, but are not limited to diethyl benzyl ammonium chloride, benzalkonium chloride, diethyl dodecyl benzyl ammonium chloride, dimethyl didodecyl ammonium chloride, octadecyl dimethyl benzyl ammonium chloride, trimethyl tetradecyl ammonium chloride, trimethyl octadecylammonium chloride, trimethyl hexadecyl ammonium chloride, Alkyl dimethyl benzyl ammonium chloride, cetyl pyridinium bromide, cetyl pyridinium chloride, dodecylpyridinium chloride, and benzyl dodecyl bis(B-hydroxyethyl)ammonium chloride.

Examples of phenolic compounds which may be used according to the present embodiments include, but are not limited to phenol, para-chlorometaxylenol, cresol and hexylresorcinol.

The antiseptic composition may also comprise other agents which may be beneficial for a subject. For example, an antibiotic or, in the case of a mouth rinse, the composition may also comprise other agents useful for dental care such as zinc chloride and fluoride derivatives.

Enriched nanostructure composition and/or antiseptic composition described above are, in some embodiments, characterized by antiseptic properties and as such can be used for disinfecting or sterilizing objects and body surfaces.

The terms “sterilizing” and “disinfecting” may be used interchangeably and refer to killing, preventing or retarding the growth of pathogens such as bacteria, fungi, amoebas, protozoas and/or viruses.

Examples of objects which can be sterilized using the compositions of the present embodiments include, but are not limited to a catheter (e.g. vascular catheter, urinary catheter, peritoneal catheter, epidural catheter and central nervous system catheter) a tube (e.g. nephrostomy tube and endrotracheal tube), a stent, an orthopedic device, a prosthetic valve, and a medical implant. Other examples include inorganic surfaces such as floors, table-tops, counter-tops, hospital equipment, wheel chairs, gauze and cotton.

Such objects are contacted with the compositions of the present embodiments for a period of time (e.g. one minute at room temperature). However, the compositions of the present embodiments should retain their antiseptic properties at higher temperatures (e.g. 50° C.) so that the objects may be heated in the presence of the antiseptic composition if required.

In order to improve sterilizing efficiency, other agents such as antiseptic agents or cleaning agents (such as a polish, a detergent or an abrasive) can be used. When the antiseptic composition is for inanimate use, the antiseptic agent may be a toxic agent or a non-toxic agent. Examples of toxic antiseptic agents include, but are not limited to formaldehyde, chlorine, mercuric chloride and ethylene oxide. Examples of non-toxic agents are detailed hereinabove.

Alternatively compositions of the present embodiments can be used for disinfecting a body surface of an individual. This can be effected by providing to the body surface of the individual in need thereof an amount of a composition of the present embodiments.

In order to improve the disinfection, other agents such as antiseptic agents, or other therapeutic agents as detailed hereinabove can be provided.

As used herein, the phrase “body surface” refers to a skin, a tooth or a mucous membrane (e.g. the mucous membrane lining the mouth). Preferably, the composition of the present embodiments does not traverse these body surfaces and enter the circulation.

As used herein, the term “individual” refers to a human or animal subject (i.e., dead or living individuals).

The antiseptic composition of the present embodiments may also comprise other physiologically acceptable carriers. Additionally, the enriched nanostructure composition of the present embodiments may also comprise an excipient or an auxiliary.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Preferably, the antiseptic composition of the present embodiments is applied locally, e.g. placed on the skin, rinsed in the mouth or gargled in the throat.

Antiseptic compositions of the present embodiments may be manufactured by processes well known in the art, e.g., by means of conventional mixing, dissolving, granulating, dragee-making, levigating, emulsifying, encapsulating, entrapping or lyophilizing processes. Manufacturing of the enriched nanostructure composition of the present embodiments is described hereinabove.

Antiseptic compositions for use in accordance with the present embodiments thus may be formulated in conventional manner. Proper formulation is dependent upon the intended use.

For example, the antiseptic composition of the present embodiments may be formulated for disinfecting the oral cavity and as such may be formulated as any oral dosage form as long as it is not deliberately swallowed. Examples of oral dosage forms include but are not limited to a mouthwash, a strip, a foam, a chewing gum, an oral spray, a capsule and a lozenge.

The antiseptic composition of the present embodiments may also be formulated as a topical or mucosal dosage form. Examples of topical or mucosal dosage forms include a cream, a spray, a wipe, a foam, a soap, an oil, a solution, a lotion, an ointment, a paste and a gel.

The antiseptic composition may be formulated as a liquid comprising at least 1% by volume of the enriched nanostructure composition. Alternatively, the antiseptic composition may be formulated as a solid or semi-solid comprising at most 0.258 gr/100 ml of the enriched nanostructure composition.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in a mixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The active ingredients for use according to the present embodiments may be conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Pharmaceutical compositions suitable for use in context of the present embodiments include compositions wherein the active ingredients are contained in an amount effective to achieve the intended purpose. More specifically, a therapeutically effective amount means an amount of active ingredients (antiseptic agent) effective to disinfect.

Determination of a therapeutically effective amount is well within the capability of those skilled in the art, especially in light of the detailed disclosure provided herein.

For any preparation used in the methods of the embodiments, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of local administrations, with course of treatment lasting from several days to several weeks or until cure is effected or diminution of the disease state is achieved.

The amount of a composition to be locally administered will, of course, be dependent on the subject being treated, the severity of the affliction, the manner of administration, the judgment of the prescribing physician, etc.

Compositions of the present embodiments may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the embodiments formulated in a compatible pharmaceutical carrier may also be prepared, placed in an appropriate container, and labeled for treatment of an indicated condition, as if further detailed above.

The present embodiments further comprises a novel cryoprotective composition and methods of using same. Specifically, the present embodiments can be used to cryopreserve cellular matter thereby facilitating its storage, transporting and handling.

Cryobiology embraces a wide range of applications and has the potential to provide solutions for the long term storage of many types of biological material. If not properly controlled, however, cryopreservation can lead to cell damage and a decrease in cell viability due to thermal, osmotic, and/or mechanical shock and the formation of crystals, which can damage cellular structures, particularly the plasma membrane. In addition, the process of freezing and thawing causes dehydration of the cell with potential for cellular damage. The use of cryoprotectants (i.e., cryoprotective agents) helps to alleviate some of these problems. Commonly used cryoprotectants include glycerol, hydroxyethyl starch (HES) ethylene glycol and DMSO. Although essential for reducing the injury of cells during freezing and thawing, these cryoprotectants are also toxic to the cell. For example, the toxic effects of glycerol on sperm cells have been reported even at concentrations of less than 2% (Tulandi and McInnes, 1984). Additionally it has been shown that sperm motility decreases as glycerol concentration increases (Weidel and Prins, 1987, J Androl., January-February; 8(1):41-7; Critser et al., 1988, Fertil Steril. August; 50(2):314-20). Furthermore, the presence of cryoprotective agents was shown to provoke sperm-cell injury due to osmotic stress (Critser et al., 1988, Fertil Steril. August; 50(2):314-20).

Therefore, it would be highly advantageous to have novel cryoprotective compositions which are devoid of the above limitations.

In some embodiments of the present invention the enriched nanostructure composition can be used to efficiently cryoprotect cellular matter.

In some embodiments of the present invention the enriched nanostructure composition in the presence of a buffer comprising a cryoprotective agent (glycerol) is more effective than the buffer alone at both protecting sperm cells following cryoprotection and at increasing sperm quality following thawing. The compositions of the present embodiments may therefore be used to reduce the amount of toxic cryoprotective agents (such as glycerol) necessary for cryoprotection, thereby limiting the cryoprotective agents' deleterious effects.

In some embodiments of the present invention there is provided a cryoprotective composition comprising enriched nanostructure composition of the present embodiments and optionally at least one cryoprotective agent.

As used herein the phrase “cryoprotective composition” refers to a liquid composition that reduces the injury of cells (e.g., mechanical injury caused by intracellular and extracellular ice crystal formation; and injury caused by osmotic forces created by changing solute conditions caused by extracellular ice formation) during freezing and thawing.

As used herein, the phrase “cryoprotective agent” refers to a chemical or a chemical solution which facilitates the process of cryoprotection by reducing the injury of cells during freezing and thawing. Preferably, the cryoprotective agent is non-toxic to the cellular matter under the conditions at which it is used (i.e. at a particular concentration, for a particular exposure time and to cells in a medium of a particular osmolarity). According to the present embodiments a cryoprotective agent may be cell permeating or non-permeating. Examples of cryoprotective agents include but are not limited to, dehydrating agents, osmotic agents and vitrification solutes (i.e., solutes that aid in the transformation of a solution to a glass rather than a crystalline solid when exposed to low temperatures).

Without being bound to theory, it is believed that non-permeating cryoprotective agents inhibit the efflux of intracellular water thereby preventing cell shrinkage beyond its minimum critical volume. By reducing cellular retraction, cryoprotective agents attenuate hyperconcentration of the intracellular fluid thereby inhibiting the precipitation of proteins. Permeating cryoprotective agents reduce the amount of ice formed therein, hence reducing the amount of physical injury to cell membranes and organelles.

Preferably, the cryoprotective agent and its concentration are selected on an empirical basis, since each cell responds to an individual cryoprotective agent in a particular way according to its type and environment. Typically, a tissue requires a more penetrating cryoprotective agent than a cell suspension. Conversely, cryoprotection of small cells may not require agents that penetrate cell membranes. In addition, the cryoprotective agent and its concentration are selected according to the method and stage of cryoprotection as further described hereinbelow.

Examples of cryoprotective agents that can be used according to the present embodiments include, but are not limited to acetamide, agarose, alginate, 1-analine, albumin, ammonium acetate, butanediol, chondroitin sulfate, chloroform, choline, dextrans, diethylene glycol, dimethyl acetamide, dimethyl formamide, dimethyl sulfoxide (DMSO), erythritol, ethanol, ethylene glycol, formamide, glucose, glycerol, alpha.-glycerophosphate, glycerol monoacetate, glycine, hydroxyethyl starch, inositol, lactose, magnesium chloride, magnesium sulfate, maltose, mannitol, mannose, methanol, methyl acetamide, methyl formamide, methyl ureas, phenol, pluronic polyols, polyethylene glycol, polyvinylpyrrolidone, proline, propylene glycol, pyridine N-oxide, ribose, serine, sodium bromide, sodium chloride, sodium iodide, sodium nitrate, sodium sulfate, sorbitol, sucrose, trehalose, triethylene glycol, trimethylamine acetate, urea, valine and xylose.

Preferably the cryoprotective composition of the present embodiments comprises less than 20% glycerol and even more preferably is devoid of glycerol (for the reasons described hereinabove).

The concentration of nanostructures is preferably selected according to the particular stage or method of cryopreservation as described herein below.

Without being bound to theory, it is believed that the long-range interactions between the nanostructures lends to the unique characteristics of the cryoprotective composition. One such characteristic is that the enriched nanostructure composition of the present embodiments is able to enhance the cryoprotective properties of other cryoprotective agents such as glycerol. This is beneficial as it enables addition of a lower concentration of glycerol (or an absence of glycerol) so that potential toxic side effects are reduced. Another characteristic is that the enriched nanostructure composition of the present embodiments enhances cryoprotective properties by providing a stabilizing environment.

Cryoprotective compositions of the present embodiments may additionally comprise one or more stabilizing agents. As used herein the phrase “stabilizing agent” refers to an agent that increases cellular viability. The stabilizing agents of the cryoprotective compositions of the present embodiments and their concentrations are selected according to the cell type and cell environment. Stabilizer concentrations are generally used at between about 1 μM to about 1 mM, or preferably at between about 10 μM to about 100 μM.

In one embodiment the stabilizing agent increases cellular viability by removing harmful substances from the culture medium. The stabilizing agent may remove both naturally occurring substances (i.e. those secreted by cells during growth or cell death) and artificially introduced substances from the culture medium. For example, a stabilizer may be a radical scavenger chemical or an anti-oxidant that neutralizes the deleterious effects attributable to the presence of active oxygen species and other free radicals. Such substances are capable of damaging cellular membranes, (both internal and external), such that cryoprotection and recovery of cellular matter is seriously compromised. If these substances are not removed or rendered otherwise ineffective, their effects on viability are cumulative over time, severely limiting practical storage life. Furthermore, as cells die or become stressed, additional harmful substances are released increasing the damage and death of neighboring cells.

Examples of oxygen radical scavengers and anti-oxidants include that may be used according to the present embodiments include but are not limited to reduced glutathione, 1,1,3,3-tetramethylurea, 1,1,3,3-tetramethyl-2-thiourea, sodium thiosulfate, silver thiosulfate, betaine, N,N-dimethylformamide, N-(2-mercaptopropionyl)glycine, .beta.-mercaptoethylamine, selenomethionine, thiourea, propylgallate, dimercaptopropanol, ascorbic acid, cysteine, sodium diethyl dithiocarbonate, spermine, spermidine, ferulic acid, sesamol, resorcinol, propylgallate, MDL-71,897, cadaverine, putrescine, 1,3- and 1,2-diaminopropane, deoxyglucose, uric acid, salicylic acid, 3- and 4-amino-1,2,4-triazol, benzoic acid, hydroxylamine and combinations and derivatives of such agents.

Stabilizing agents which may be useful in the cryoprotection of plant cell may include agents that hinder or substantially prevent ethylene biosynthesis and/or ethylene action. It is well known that plant cells emit toxic ethylene when stressed. Therefore, prevention of either the generation of ethylene or the action of ethylene will further enhance cell viability and cell recovery from the cryoprotection process.

Examples of ethylene biosynthetic inhibitors that can be used in the present embodiments include, but are not limited to Rhizobitoxin, Methoxylamine Hydrochloric acid, Hydroxylamine Analogs, alpha.-Canaline, DNP (2,4-SDS (sodium lauryl sulfate) dinitrophenol), Triton X-100, Tween 20, Spermine, Spermidine, ACC Analogs, alpha.-Aminoisobutyric Acid, n-Propyl Gallate, Benzoic Acid, Benzoic Acid Derivatives, Ferulic Acid, Salicylic Acid, Salicylic Acid Derivatives, Sesamol, Cadaverine, Hydroquinone, Alar AMO-1618, BHA (butylated hydroxyanisol), Phenylethylamine, Brassinosteroids, P-chloromercuribenzoate, N-ethylmaleimide, Iodoacetate, Cobalt, Chloride and other salts, Bipyridyl Amino (oxyacetic) Mercuric Chloride and other Acid salts, Salicyl alcohol, Salicin, Nickle, Chloride and other salts, Catechol, Pffloroglucinol, 1,2-Diaminopropane, Desferrioxamine Indomethacin 1,3-Diaminopropane

Examples of inhibitors of ethylene action include but are not limited to Silver Salts, Benzylisothiocyanate, 8-Hydroxyquinoline sulfate, 8-Hydroxyquinoline citrate, 2,5-norbornadiene, N-ethoxycarbonyl-2-ethoxy-1,2-dihydroquinoline, Trans-cyclooctene, 7-Bromo-5-chloro-8-hydroxyquinoline, Cis-Propenylphosphonic Acid, Diazocyclopentadiene, Methylcyclopropane, 2-Methylcyclopropane, Carboxylic Acid, Methylcyclopropane carboxylate, Cyclooctadiene, Cyclooctadine (Chloromethyl) and Cyclopropane

Silver ions are also potent anti-ethylene agent in various plants and are known to improve the longevity of plant tissues and cell cultures. Examples of silver salts which may be used according to the present embodiments include Silver Thiosulfate, Silver Nitrate, Silver Chloride, Silver Acetate, Silver Phosphate, Citric Acid Tri-Silver Salt, Silver Benzoate, Silver Sulfate, Silver Oxide, Silver Nitrite, Silver Cyanate, Lactic Acid Silver Salt and Silver Salts of Pentafluoropropionate Hexafluorophosphate and Toluenesulfonic Acid.

In another embodiment, the stabilizing agent increases cellular viability by stabilizing the cell membrane e.g. by intercalating into the lipid bilayer (e.g. sterols, phospholipids, glycolipids, glycoproteins) or stabilizing membrane proteins (e.g. divalent cations). Examples of divalent cations that may be used in the cryoprotective composition of the present embodiments include, but are not limited to CaCl₂, MnCl₂ and MgCl₂. Sodium is less preferred due to its toxicity at any more than trace concentrations. Preferred concentrations range from about 1 mM to about 30 mM, and more preferably from about 5 mM to about 20 mM and still more preferably at about 10 mM or 15 mM. Divalent cations also reduce freezing temperatures and allows for the more rapid passage of cells through freezing points.

In yet another embodiment, the stabilizing agent increases cellular viability by preventing or minimizing heat-shock. Thus the stabilizing agent may be a heat shock protein or may be a heat-shock protein stabilizer (e.g. a divalent cation, as described hereinabove).

The cryoprotective composition of the present embodiments may further comprise stabilizers such as growth factors, egg yolk, serum (e.g. fetal calf serum) and antibiotic compounds (e.g. tylosin, gentamicin, lincospectin, and/or spectinomycin).

In addition, the cryoprotective composition of the present embodiments may comprise growth medium or buffer. The type of media or buffer selected is dependent on the cell type being cryoprotected, and examples are well known in the art. Suitable examples of acceptable cell buffers include phosphate based buffers such as PBS and Tris based buffers such as Tris EDTA. An example of a growth medium that may be added to the cryoprotective composition of the present embodiments is DMEM.

As mentioned hereinabove, the compositions of the present embodiments are characterized by cryoprotective properties and as such can be used for cryopreserving cellular matter.

Thus, according to an aspect of some embodiments of the present invention there is provided a method of cryopreserving cellular matter comprising: (a) contacting the cellular matter with the enriched nanostructure composition of the present embodiments; and (b) subjecting the cellular matter to a cryopreserving temperature.

As used herein, the term “cryopreserving” refers to maintaining or preserving the viability of cellular matter by storing at very low temperatures. Typically, cryopreserving is effected in the presence of a cryoprotective agent. Preferably cellular matter may be cryopreserved for at least five years following the teachings of the present embodiments.

As used herein, the phrase “cellular matter” refers to a biological material that comprises cells.

Examples of cellular matter which may be cryopreserved according to the present embodiments include prokaryotic and eukaryotic cellular matter (e.g., mammalian, plant, yeast), but are not limited to, a cellular body fluid (e.g., spinal fluid, blood, amniotic fluid, saliva, synovial fluid, vaginal secretions and semen), isolated cells, a cell culture (e.g., cell-line, primary cell culture, yeast or bacteria culture), a cell suspension, immobilized cells, (e.g. scaffold associated), a tissue, an organ or an organism.

Examples of plant cellular matter include but are not limited to growth needles, leaves, roots, barks, stems, rhizomes, callus cells, protoplasts, cell suspensions, organs, meristems, seeds and embryos, as well as portions thereof.

In a particular embodiment, the cellular matter may comprise stem cells, sperms cells or eggs (i.e. oocytes).

In another particular embodiment, the cellular matter may be naïve or genetically modified.

Cellular matter may be obtained from a living organism or cadaver. For example it may be obtained by surgery (e.g., biopsy) or in an ejaculate. Alternatively, cellular matter may be obtained from a laboratory cell culture.

The following summarizes typical cryopreservation procedures for exemplary cellular matter.

### Semen

Semen may be obtained from normal, oligospermic, teratospermic or asthenozoospermic males preferably by donation, although it may also be obtained by surgical methods. The sperm is typically subjected to functional tests in order to determine the quantity of sample that is required to be cryopreserved if there is to be a realistic chance of fertilization following recovery. Semen samples are typically mixed in a 1:1 ratio with the cryoprotecting composition of the present embodiments, and frozen in 0.5 ml aliquots in straws using static vapour phase cooling.

### Embryo

Embryos are typically cryopreserved at the pre-implantation stage (e.g. blastocyst stage) following in-vitro fertilization. Embryos are selected according to a range of criteria in order to optimize successful cryopreservation (e.g. 1. blastocyst growth rate—growth rate at day 5 should be greater than growth rate at day 6, which in turn should be greater than the growth rate at day 7; 2. overall cell number—number should be greater or equal to 60 cells (depending on the day of development); 3. relative cell allocation to trophectoderm: inner cell mass; 4. blastomere regularity; 5. mononucleation and; 6. DNA fragmentation).

Standard embryo cryopreservation techniques may involve exposing the embryo to the cryoprotecting composition of the present embodiments diluted in a simple sodium-based salt solution for 5-15 minutes to allow uptake. The embryos may then cooled quickly (−2° C./min) to about 7° C. at which point they may be seeded, cooled slowly (−0.3° C. to −0.5° C./min) to about −30° C. or below, and then plunged directly into liquid nitrogen. A programmable freezer is typically required for controlled rate cooling. The embryos may be thawed using a rapid approach. Embryos can also be rapidly frozen or vitrified, but only using very elevated cryopreservative concentrations (2M to 6M) that are toxic to cells when they are exposed for more than a few minutes.

### Oocytes

Preferably, oocytes that are used for cryopreservation are mature. Mature oocytes may be removed by surgical procedures. Oocyte stimulation prior to removal may also be required. Typically oocytes are selected for cryopreservation based on the following criteria; translucence, shape and extrusion of the first polar body. Typical protocols for the cryopreservation of oocytes are described in U.S. Pat. No. 6,500,608 and U.S. Pat. No. 5,985,538.

### Stem Cells

Preservation of pluripotent stem cells poses additional challenges to cryobiology since not only must the cells remain viable, but they must also retain their differentiative capacity (i.e., be maintained in an undifferentiated state). Thus, certain signal transduction pathways must remain in place, and the stresses associated with freezing and drying must not induce premature or erroneous differentiation. Stabilizers may be included which maintain the differentiationless phenotype of the cells immediately following thawing.

Typically stem cell cryopreservation protocols include (1) conventional slow-cooling protocols applied to adherent stem cell colonies and (2) vitrification protocols for both adherent stem cell colonies and freely suspended stem cell clumps.

### Skin

Skin is typically removed from cadavers or healthy individuals. Animal skin tissue may also be cryopreserved for use in grafting. The skin is typically tissue-typed prior to cryopreservation or following thawing. Skin cells may be cultured and expanded in vitro prior to cryopreservation. Cryopreservation typically requires a fast thaw protocol. The success or failure of the protocol is measured either by graft take to a wound bed or by a cell viability assay.

### Ovarian Tissue

Ovarian tissue (whole ovary or a portion thereof) may be removed from healthy or non-healthy women. Examples of diseases in which it may be advantageous to cryopreserve ovarian tissue include cancer, malignant diseases such as thalassemia and certain auto-immune conditions. Healthy women who have a history of early menopause may also desire ovarian tissue cryopreservation. Following removal or thawing, the tissue may be screened for malignant cells, and assessed for safety for subsequent auto-grafting.

The cellular matter may be conditioned to facilitate the cryoprotection procedure or may be contacted directly with the compositions of the present embodiments. As used herein the term “conditioning” refers to protecting the cellular matter from the toxic effects of nanostructures and/or cryoprotecting agents and/or the toxic effects of a decreased temperature. For example the cellular matter may be conditioned with stabilizers and subsequently incubated in the presence of the compositions of the present embodiments. Alternatively, the compositions of the present embodiments may be initially applied to the cells followed by the addition of stabilizers or other cryoprotective agents.

Examples of stabilizers are described hereinabove.

Additionally or alternatively, the cellular matter may be cold acclimatized prior to cryoprotecting. This may be affected simultaneously or following conditioning with stabilizers and either prior to or simultaneously with incubating with the compositions of the present embodiments. This prepares cells for the cryopreservation process by significantly retarding cellular metabolism and reducing the shock of rapid temperature transitions through some of the more critical temperature changes. Critical temperature ranges are those ranges at which there is the highest risk of cell damage, for example, around the critical temperatures of ice crystal formation. As known to those of ordinary skill in the art, these temperatures vary somewhat depending upon the composition of the solution. (For water, the principal component of most cell culture mediums, ice crystal formation and reformation occur at about 0° C. to about −50° C.).

Acclimation results in the accumulation of endogenous solutes that decreases the extent of cell dehydration at any given osmotic potential, and contributes to the stabilization of proteins and membranes during extreme dehydration.

Acclimation may be carried out in a stepwise fashion or gradually. Steps may be in decreasing increments of about 0.5° C. to about 10° C. for a period of time sufficient to allow the cells acclimate to the lower temperature without causing damage. The temperature gradient, whether gradual or stepwise, is scaled to have cells pass through freezing points as quickly as possible. Preferably, acclimation temperatures are between about 1° C. to about 15° C., more preferably between about 2° C. to about 10° C. and even more preferably about 4° C. Cells may be gradually, in a step-wise or continuous manner, or rapidly acclimated to the reduced temperature. Techniques for acclimation are well known to those of ordinary skill and include commercially available acclimators. Gradual acclimation comprises reducing incubation temperatures about 1° C. per hour until the target temperature is achieved. Gradual acclimation is most useful for those cells considered to be most sensitive and difficult to cryoprotect. Stepwise acclimation comprises placing the cells in a reduced temperature for a period of time, a subsequently placing in a further reduced temperature for another period of time. These steps may be repeated as required.

Lyophilization of cellular matter may also be performed prior to cryoprotection. Lyophilization is directed to reducing the water content of the cells by vacuum evaporation. Vacuum evaporation involves placing the cells in an environment with reduced air pressure. Depending on the rate of water removal desired, the reduced ambient pressure operating at temperatures of between about −30° C. to −50° C. may be at 100 torr, 1 torr, 0.01 torr or less. Under conditions of reduced pressure, the rate of water evaporation is increased such that up to 65% of the water in a cell can be removed overnight. With optimal conditions, water removal can be accomplished in a few hours or less. Heat loss during evaporation maintains the cells in a chilled state. By careful adjustment of the vacuum level, the cells may be maintained at a cold acclimation temperature during the vacuum evaporation process. A strong vacuum, while allowing rapid water removal exposes the cells to the danger of freezing.

Freezing may be controlled by applying heat to the cells directly or by adjustment of the vacuum level. When the cells are initially placed in the evaporative chamber, a high vacuum may be applied because the residue heat in the cells will prevent freezing. As dehydration proceeds and the cell temperature drops, the vacuum may be decreased or heating may be applied to prevent freezing. The semi-dry cells may have a tendency to scatter in an evaporative chamber. This tendency is especially high at the end of the treatment when an airstream is allowed back into the chamber. If the air stream proximates the semi-dry cells, it may cause the cells to become airborne and cause cross contamination of the samples. To prevent such disruptions, evaporative cooling may be performed in a vacuum centrifuge wherein the cells are confined to a tube by centrifugal force while drying. The amount of water removed in the process may be monitored periodically by taking dry weight measurement of the cells.

Heat shock treatment may also be performed as an alternative to acclimation prior to cryoprotection. Heat-shock treatment is known to induce de novo synthesis of certain proteins (heat-shock proteins) that are supposed to be involved in adaptation to stress. In addition, heat-shock treatment acts to stabilize membranes and proteins. It tends to improve the survival of cells following cryopreservation by about 20% to about 40%. This procedure involves the incubation of cellular matter (either conditioned or not) in a water-bath shaker at between about 31° C. to about 45° C. preferably between about 33° C. to about 40° C. and more preferably at about 37° C. Culturing is performed from a few minutes to a few hours, preferably from about one hour to about six hours, and more preferably from about two hours to about four hours.

As mentioned hereinabove, the method of the present embodiments is effected by contacting (incubating) the cellular matter with the compositions of the present embodiments. Preferably, the contacting acts to equilibrate intracellular and/or extracellular concentrations of the nanostructures. The composition of the present embodiments may be added directly to the cellular matter or may be diluted into the medium where the cellular matter is being incubated. To minimize the time required for equilibration, contacting may be performed at about room temperature, although optimal temperature and other conditions for loading will preferably match conditions such as medium, light intensities and oxygen levels that maintain a cell viable.

The compositions of the present embodiments may be applied directly to the cellular matter or may be diluted in cellular matter incubating mediums, such as culture mediums. Additionally a stepwise incubation (contacting) may be effected. Thus for example, stepwise contacting can be effected such that the cellular matter is incubated in the presence of an increasing concentration of nanostructures. Thus, for example, the cellular matter may be initially contacted with a composition comprising 10¹⁰ nanostructures per liter and finally contacted with a composition comprising 10¹⁵ nanostructures per liter.

Stepwise contacting is sometimes desired to facilitate delivery of the nanostructures to cells as it is somewhat gentler than single dose loading. Time increments or interval between additions for stepwise loading may range from minutes to hours or more, but are preferable from about one to about ten minutes, more preferably from about one to about five minutes and still more preferably about one or about two minute intervals. The numbers of additions in a stepwise contacting procedure is typically whatever is practical and can range from very few to a large plurality. Preferably, there are less than about twenty additions, more preferably less than about ten and even more preferably about five. Interval periods and numbers of intervals are easily determined by one of ordinary skill in the art for a particular type of cell and loading agent. Incubation times range from minutes to hours as practical.

The cryoprotecting agents or nanostructures in the composition of the present embodiments may be at a high enough concentration, such that contacting triggers vitrification of the cellular matter.

Vitrification procedures involve gradual or stepwise osmotic dehydration of the cellular matter by direct exposure to concentrated solutions prior to quenching in liquid nitrogen.

Prior to vitrifying, the cellular matter may be incubated with the compositions of the present embodiments wherein their concentration is not high enough to bring about vitrification. This primarily serves to prevent dehydration-induced destabilization of cellular membranes and possibly proteins. These compositions may optionally be removed prior to vitrification. If the composition remains, the concentration of nanostructures may be increased either gradually or in a stepwise fashion to facilitate vitrification. Other cryoprotecting agents apart from those used to initially contact the cellular matter may be added, or alternatively the identical agents may be added, but at higher concentrations, also in a step-wise or gradual fashion as discussed hereinabove. Concentrations of cryoprotecting agents may range from about 4 M to about 10 M, or between about 25% to about 60%, by weight. This produces an extreme dehydration of the sample cells. Solutions in excess of 7 M typically remove more than 90% of the osmotically active water from the cells; however, precise concentrations for each agent can be empirically determined. Cryoprotecting agents which may be used for vitrification include DMSO, propylene glycol, mannitol, glycerol, polyethylene glycol, ethylene glycol, butanediol, formamide, propanediol and mixtures of these substances.

To minimize the injurious consequences of exposure to high concentrations of cryoprotecting agents or nanostructures, dehydration may be performed at about 0° C. to about 4° C. with the time of exposure as brief as possible. Under these conditions, there is no appreciable influx of additional cryoprotecting agents into the cellular matter because of the difference in the permeability coefficient for water and solutes. As a result, the cellular matter remains contracted and the increase in cytosolic concentration required for vitrification is attained by dehydration.

Cellular matter which has been contacted with compositions of the present embodiments is cryopreserved by freezing to cryopreservation temperatures. The rate of freezing must strike a balance between the damage caused to cells by mechanical forces during quick freezing and the damage caused to cells by osmotic forces during slow freezing. Different optimal cooling rates have been described for different cells. It has been suggested that the different optimal cooling rates are due to the differences in cellular ice nucleation constants and in phase transition temperature of the cell membrane for different cell types (PCT Publication No. WO 98/14058; Karlsson et al., Biophysical J 65: 2524-2536, 1993). Freezing rates between −1° C. per minute and −10° C. per minute are preferred in the art (Karlsson et al., Biophysical J 65: 2524-2536, 1993). Freezing should be sufficiently rapid to inhibit ice crystal formation. The freezing time should be around 5 minutes or 4 minutes, 3 minutes, 2 minutes, or one minute or less. The critical freezing time should be measured from the frame of reference of a single cell. For example, it may take 10 minutes to pour a large sample of cells into liquid nitrogen, however the individual cell is frozen rapidly by this method.

As mentioned above, the cellular matter may be vitrified. Under those conditions, the cellular matter may be cooled at extremely rapid rates (supercooling) without undergoing intercellular or intracellular ice formation. As well as obviating all of the factors that affect ice formation, rapid cooling also circumvents problems of chilling sensitivity of some cellular matter.

Cellular matter may be directly frozen. Direct freezing methods include dripping, spraying, injecting or pouring cells directly into a cryogenic temperature fluid such as liquid nitrogen or liquid helium. Cellular matter may also be directly contacted to a chilled solid, such as a liquid nitrogen frozen steel block. The cryogenic temperature fluid may also be poured directly onto the cellular matter. The direct method also encompasses contact cells with gases, including air, at a cryogenic temperature. A cryogenic gas stream of nitrogen or helium may be blown directly over or bubbled into a cell suspension. Indirect method involved placing the cells in a container and contacting the container with a solid, liquid, or gas at cryogenic temperature. Examples of containers include plastic vials, glass vials, ampules which are designed to withstand cryogenic temperatures. The container for the indirect freezing method does not have to be impermeable to air or liquid. For example, a plastic bag or aluminum foil is adequate. Furthermore, the container may not necessarily be able to withstand cryogenic temperatures. A plastic vial which cracks but remain substantially intact under cryogenic temperatures may also be used. Cells may also be frozen by placing a sample of cells on one side of a metal foil while contacting the other side of the foil with a gas, solid, or liquid at cryogenic temperature.

Compositions of the present embodiments may be included in containers suitable for cryopreservation. The container is preferably impervious to the chemicals which it is designed to withhold—for example nanostructures and additional cryoprotecting agents as discussed herein below. The container is preferably made of a material that can withstand cryogenic temperatures. Preferably the container is flexible so that it can absorb volume changes of the various components during the freeze/thaw cycles. Even more preferably, the container of the present embodiments comprises an open tube.

Cryopreserved cellular matter may be maintained at temperatures appropriate for cryo-storage. Final storage temperature is dependent on cell type, but is generally known in the art to be approximately −80° C. to −196° C., the temperatures maintained by dry ice and liquid nitrogen freezers, respectively. Preferably, cells are maintained in liquid nitrogen (about −196° C.), liquid argon, liquid helium or liquid hydrogen. These temperatures will be most appropriate for long term storage of cells, and further, temperature variations can be minimized. Long term storage may be for months and preferably for many years without significant loss of cell viability upon recovery. Short term storage, storage for less than a few months, may also be desired wherein storage temperatures of −150° C., −100° C. or even −50° C. may be used. Dry ice (carbon dioxide) and commercial freezers may be used to maintain such temperatures.

Suitable thawing and recovery is essential to cell survival and to recovery of cells in a condition substantially the same as the condition in which they were originally frozen. As the temperature of the cryoprotected cellular matter is increased during thawing, small ice crystals consolidate and increase in size. Large intracellular ice crystals are generally detrimental to cell survival. To prevent this from occurring, cryoprotected cellular matter should be thawed as rapidly as possible. The rate of heating may be at least about 30° C. per minute to 60° C. per minute. More rapid heating rates of 90° C. per minute, 140° C. per minute to 200° C. or more per minute can also be used. While rapid heating is desired, most cells have a reduced ability to survive incubation temperature significantly above room temperature. To prevent overheating, the cell temperature is preferably monitored. Any heating method can be employed including conduction, convection, radiation, electromagnetic radiations or combinations thereof. Conduction methods involve immersion in water baths, placement in heat blocks or direct placement in open flame. Convection methods involve the use of a heat gun or an oven. Radiation methods involve, for example, heat lamps or ovens such as convection or radiation ovens. Electromagnetic radiation involves the use of microwave ovens and similar devices. Some devices may heat by a combination of methods. For example, an oven heats by convection and by radiation. Heating is preferably terminated as soon as the cells and the surrounding solutions are in liquid form, which should be above 0° C. Since the cryoprotected cellular matter is frozen in the presence of nanostructures and possibly other agents that depress the freezing point, the frozen cells may liquify at a temperature below 0° C. such as at about −10° C. −20° C. −30° C. or −40° C. Thawing of the cryoprotected cells may be terminated at any of these temperatures or at a temperature above 0° C.

Dilution of the enriched nanostructure composition of the present embodiments and its subsequent removal is typically performed as rapidly as possible and as soon as possible following thawing of the cryoprotected cellular matter. If there is a high concentration of nanostructures or cryoprotecting agent in the composition, it is preferred to effect the dilution of the suspending medium while minimizing osmotic expansion. Therefore, dilution of the suspending medium and efflux of the nanostructures or other cryoprotecting agent from within the cellular matter may be accomplished by dilution in a hypertonic medium or a step-wise dilution.

Thawed cells can be gradually acclimated to conditions that allow cells to function normally or if the cellular matter is to be grown following thawing conditions that encourage growth. Cryoprotecting agents may be cytotoxic, cytostatic or mutagenic, and are preferably removed from the thawed cellular matter at a rate which would not harm the cells. A number of removal methods may be used such as resuspension and centrifugation, dialysis, serial washing, bioremediation and neutralization with chemicals, or electromagnetic radiation. The rapid removal of nanostructures and other cryoprotecting agents may increase cell stress and death and thus the removal step may have to be gradual. Removal rates may be controlled by serial washing with solutions that contain less nanostructures or cryoprotecting agents.

Thawing and post-thaw treatments may be performed in the presence of stabilizers (as described hereinabove) to ensure survival and minimize genetic and cellular damage. The stabilizers such as, for example, divalent cations or ethylene inhibitors, reduce, eliminate or neutralize damaging agents which results from cryopreservation. Such damaging agents include free radicals, oxidizers and ethylene.

Preferably, the cellular matter comprises fully-functioning cells so as to increase the percentage of cells that survive following thawing. It is expected that abnormal sperm cells which had a low pregnancy potential, will have a decreased survival rate following freezing stress in the presence of the cryoprotective composition of the present embodiments than normal sperm cells. Thus, cryoprotecting a mixture of functioning and non-functioning sperm cellular matter in compositions of the present embodiments may increase the ratio of functioning: non-functioning cells, thereby improving chances of fertilization following thawing.

Preferably at least 10% of the cells in the cellular matter are fully functioning and viable (e.g. sperm cells should be motile, capable of fertilizing an oocyte and should not comprise fragmented DNA) and more preferably 20%, more preferably 30%, more preferably 40%, more preferably 50%, more preferably 60%, more preferably 70%, more preferably 80%, and even more preferably 90%.

After thawing, the cellular matter may optionally be assayed for viability or may be used immediately for transplantation. Viability may be determined by histological and functional methods. Cells are assayed by histological methods known in the art, including, for example, morphological index, exclusion of vital stains, and intracellular pH.

One or more in vitro assays are preferably used to establish functionality of cellular matter. Assays or diagnostic tests well known in the art can be used for these purposes. See, e.g., METHODS IN ENZYMOLOGY, (Abelson, Ed.), Academic Press, 1993. For example, an ELISA (enzyme-linked immunosorbent assay), chromatographic or enzymatic assay, or bioassay specific for the secreted product can be used.

Specifically, if the cellular matter contains sperm, its condition may be analyzed by wave motion analysis, motility assays, and viability counts. For example, a gross microscopic analysis of the semen can be conducted by analyzing wave motion under low magnification (e.g. 10 fold) and ascribing a score for motion from 0-5, with 0 being no wave motion and 5 being rapid wave motion with eddies. Secondly, under higher magnification (e.g. 40 fold) the number of motile sperm can be counted and scored as a percentage of total sperm. This percentage is later multiplied by the concentration/count to determine the number of visibly viable sperm. Sperm concentration can be determined by various procedures: a microcuvette containing semen diluted 1:10 with 0.9% saline is assayed in a Spermacue photometer; or a series of dilutions (1:1000) of the sperm are made and counted with a hemocytometer.

The percentage of viable sperm ratio can be determined by placing a 15 μl drop of extended sample of sperm on a microscope slide with a 15 lilldrop of a Live/Dead stain (Morphology Stain, Lane Manufacturing, Inc., Denver Colo.). A thin smear is prepared after mixing the two drops. The sample is air dried, and then 200 individual sperm are counted by staining with the vital dye under the microscope with a 100 fold oil immersion lens.

A sperm's integrity can be assayed by observation of the sperm's acrosomal cap and tail morphology using the Spermac stain. Another microscope slide is prepared with a 15 μl drop of sperm, air dried, and then stained with Spermac following the manufacturer's specification. The overall quality and morphology of the sample is determined by scoring acrosomal caps as intact or non-intact and by counting the number normal tails per 200 individual sperm.

In some embodiments of the present invention the enriched nanostructure composition of the present embodiments enhances the in-vivo uptake of pharmaceutical agents

The development of many pharmaceutical agents with low bioavailability such as peptides, proteins and nucleic acids has created a need to develop new and effective approaches of delivering such macromolecules to their appropriate cellular targets. Therapeutics based on either the use of specific polypeptide growth factors or specific genes to replace or supplement absent or defective genes are examples of therapeutics that require such new delivery systems. Therapeutic agents involving oligonucleotides such that they interact with DNA to modulate the expression of a gene may also require a delivery system that is capable of enhancing in vivo uptake across cellular membranes. Clinical application of such therapies depends not only on the reliability and efficiency of new delivery systems but also on their safety and on the ease with which the technologies underlying these systems can be adapted for large-scale pharmaceutical production, storage, and distribution of the therapeutic formulations.

Nanoparticle technology has found application in a variety of disciplines, but has only minimal application in pharmacology and drug delivery. Nanoparticles have been proposed as carriers of anticancer and other drugs [Couvreur et al., (1982) J. Pharm. Sci., 71: 790-92]. Other attempts have pursued the use of nanoparticles for treatment of specific disorders [Labhasetwar et al., (1997) Adv. Drug. Del. Rev., 24: 63-85]. Typically, the nanoparticles are loaded with the pharmaceutical agent.

Although nanoparticles have shown promise as useful tools for drug delivery systems, many problems remain. Some unsolved problems relate to the loading of particles with therapeutics. Additionally, the bioavailability of loaded nanoparticles is reduced since nanoparticles are taken up by cell of the reticuloendothelial system (RES). Therefore, it would be highly advantageous to have a nanoparticle delivery system which is devoid of the above limitations.

In some embodiments of the present invention the enriched nanostructure composition enhances in vivo penetration of a therapeutic agent through cell membranes. For example, a enriched nanostructure composition of the present embodiments can enhance penetration of a therapeutic agent through the skin. Additionally, the enriched nanostructure composition of the present embodiments can enhance uptake of an antibiotic agent into bacteria cells, thereby increasing its bioavailability.

Thus, according to an aspect of some embodiments of the present invention there is provided a pharmaceutical composition comprising at least one pharmaceutical agent as an active ingredient and the enriched nanostructure composition of the present embodiments.

As used herein the phrase “pharmaceutical agent as an active ingredient” refers to a therapeutic, cosmetic or diagnostic agent which is accountable for the biological effect of the pharmaceutical composition.

As used herein a “pharmaceutical composition” refers to a preparation of one or more of the active ingredients with the enriched nanostructure composition, both described herein.

In some embodiments of the present invention the nanostructures the enriched nanostructure composition is formulated to enhance in vivo uptake of the pharmaceutical agent. Without being bound to theory, it is believed that the long-range interactions between the nanostructures lends to the unique characteristics of the pharmaceutical compositions of the present embodiments. One such characteristic is that the enriched nanostructure composition of the present embodiments is hydrophobic and is thus able to enhance penetration of an active agent through cellular membranes membrane. For example, the enriched nanostructure composition of the present embodiments can enhance nucleotide uptake into cells, phage uptake and/or antibiotic uptake into bacterial cells.

Thus, the enriched nanostructure composition of the present embodiments may be formulated to enhance penetration through any biological barrier such as a cell membrane, an organelle membrane, a blood barrier or a tissue. For example the enriched nanostructure composition of the present embodiments may be formulated to penetrate the skin.

The pharmaceutical agent of the present embodiments may be a therapeutic agent, a cosmetic agent or a diagnostic agent, as further detailed hereinabove.

Examples of structural classes of therapeutic agents include, but are not limited to, inorganic or organic compounds; small molecules (i.e., less than 1000 Daltons) or large molecules (i.e., above 1000 Daltons); biomolecules (e.g. proteinaceous molecules, including, but not limited to, protein (e.g. enzymes or hormones) peptide, polypeptide, post-translationally modified protein, antibodies etc.) or nucleic acid molecules (e.g. double-stranded DNA, single-stranded DNA, double-stranded RNA, single-stranded RNA, or triple helix nucleic acid molecules) or chemicals. Therapeutic agents may be cellular agents derived from any known organism (including, but not limited to, animals, plants, bacteria, fungi, protista or viruses) or from a library of synthetic molecules. An example of a viral therapeutic cellular agent is a bacteriophage. In some embodiments of the present invention the enriched nanostructure composition enables increased bacteriophage uptake into bacteria.

Examples of therapeutic agents which may be particularly useful in treating a brain condition include, but are not limited to antibiotic agents, anti-neoplastic agents, anti-inflammatory agents, antiparasitic agents, antifungal agents, antimycobacterial agents, antiviral agents, anticoagulant agents, radiotherapeutic agents, chemotherapeutic agents, cytotoxic agents, vasodilating agents, anti-oxidants, analeptic agents, anti-convulsant agents, antihistamine agents, neurotrophic agents, psychotherapeutic agents, anxiolytic sedative agents, stimulant agents, sedative agents, analgesic agents, anesthetic agents, birth control agents, neurotransmitter agents, neurotransmitter analog agents, scavenging agents and fertility-enhancing agents.

Examples of neurotransmitter agents which can be used in accordance with the present invention include but are not limited to acetylcholine, dopamine, norepinephrine, serotonin, histamine, epinephrine, Gamma-aminobutyric acid (GABA), glycine, glutamate, adenosine, inosine and aspartate.

Neurotransmitter analog agents include neurotransmitter agonists and antagonists. Examples of neurotransmitter agonists that can be used in the present invention include, but are not limited to almotriptan, aniracetam, atomoxetine, benserazide, bromocriptine, bupropion, cabergoline, citalopram, clomipramine, desipramine, diazepam, dihydroergotamine, doxepin duloxetine, eletriptan, escitalopram, fluvoxamine, gabapentin, imipramine, moclobemide, naratriptan, nefazodone, nefiracetam acamprosate, nicergoline, nortryptiline, paroxetine, pergolide, pramipexole, rizatriptan, ropinirole, sertraline, sibutramine, sumatriptan, tiagabine, trazodone, venlafaxine, and zolmitriptan.

Examples of neurotransmitter antagonist agents that can be used in the present invention include, but are not limited to 6 hydroxydopamine, phentolamine, rauwolfa alkaloid, eticlopride, sulpiride, atropine, promazine, scopolamine, galanin, chlorpheniramine, cyproheptadine, diphenylhydramine, methysergide, olanzapine, citalopram, fluoxetine, fluvoxamine, ketanserin, oridanzetron, p chlophenylalanine, paroxetine, sertraline and venlafaxine.

Particularly useful in the present embodiments are therapeutic agents such as peptides (e.g., neuropeptides) which have specific effects in the body but which under normal conditions poorly penetrate a cell membrane or blood barrier. In addition bacteria (e.g. gram negative bacteria) may build up resistance to antibiotics such as aminoglycosides, β lactams and quinolones by making their cell membrane less permeable. Addition of the enriched nanostructure composition of the present embodiments may increase in vivo uptake into these bacteria, thereby enhancing the effectivity of the antibiotic therapeutic agent. Another example where the enriched nanostructure composition of the present embodiments may be particularly useful is together with chelation agents such as EDTA for the treatment of high blood pressure, heart failure and atherosclerosis. The chelation agent is responsible for removing Calcium from arterial plaques. However, the arterial cellular membranes are relatively impermeable to chelating agents. Thus by incorporating the enriched nanostructure composition of the present embodiments together with chelating agents, their bioavailability would be greatly enhanced.

The term “neuropeptides” as used herein, includes peptide hormones, peptide growth factors and other peptides. Examples of neuropeptides which can be used in accordance with the present invention include, but are not limited to Oxytocin, Vasopressin, Corticotropin releasing hormone (CRH), Growth hormone releasing hormone (GHRH), Luteinizing hormone releasing hormone (LHRH), Somatostatin growth hormone release inhibiting hormone, Thyrotropin releasing hormone (TRH), Neurokinin a (substance K), Neurokinin β, Neuropeptide K, Substance P, β-endorphin, Dynorphin, Met- and leu-enkephalin, Neuropeptide tyrosine (NPY), Pancreatic polypeptide, Peptide tyrosine-tyrosine (PYY), Glycogen-like peptide-1 (GLP-1), Peptide histidine isoleucine (PHI), Pituitary adenylate cyclase activating peptide (PACAP), Vasoactive intestinal polypeptide (VIP), Brain natriuretic peptide, Calcitonin gene-related peptide (CGRP) (α- and β-form), Cholecystokinin (CCK), Galanin, Islet amyloid polypeptide (IAPP), Melanin concentrating hormone (MCH), Melanocortins (ACTH, α-MSH and others), Neuropeptide FF, Neurotensin, Parathyroid hormone related protein, Agouti gene-related protein (AGRP), Cocaine and amphetamine regulated transcript (CART)/peptide, Endomorphin-1 and -2, 5-HT-moduline, Hypocretins/orexins Nociceptine/orphanin FQ, Nocistatin, Prolactin releasing peptide, Secretoneurin and Urocortin

In some embodiments of the present invention the enriched nanostructure composition is used to enhance in vivo delivery of diagnostic agents. Examples of diagnostic agents which can be used in accordance with the present embodiments include the x-ray imaging agents, fluorescent imaging agents and contrast media. Examples of x-ray imaging agents include WIN-8883 (ethyl 3,5-diacetamido-2,4,6-triiodobenzoate) also known as the ethyl ester of diatrizoic acid (EEDA), WIN 67722, i.e., (6-ethoxy-6-oxohexyl-3,5-bis(acetamido)-2,4,6-triiodobenzoate; ethyl-2-(3,5-bis(acetamido)-2,4,6-triiodo-benzoyloxy)butyrate (WIN 16318); ethyl diatrizoxyacetate (WIN 12901); ethyl 2-(3,5-bis(acetamido)-2,4,6-triiodobenzoyloxy)propionate (WIN 16923); N-ethyl 2-(3,5-bis(acetamido)-2,4,6-triiodobenzoyloxy acetamide (WIN 65312); isopropyl 2-(3,5-bis(acetamido)-2,4,6-triiodobenzoyloxy)acetamide (WIN 12855); diethyl 2-(3,5-bis(acetamido)-2,4,6-triiodobenzoyloxy malonate (WIN 67721); ethyl 2-(3,5-bis(acetamido)-2,4,6-triiodobenzoyloxy)phenylacetate (WIN 67585); propanedioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo-4-(ethyl-3-ethoxy-2-butenoate) ester (WIN 68209). Other contrast media include, but are not limited to, magnetic resonance imaging aids such as gadolinium chelates, or other paramagnetic contrast agents. Examples of such compounds are gadopentetate dimeglumine (Magnevist®) and gadoleridol (Prohance®). Patent Application No. 20010001279 describes liposome comprising microbubbles which can be used as ultrasound contrast agents. Thus, diagnostic contrast agents can also be used in corporation with the present invention for aiding in ultrasound imaging of the brain.

Labeled antibodies may also be used as diagnostic agents according to various exemplary embodiments of the present invention. Use of labeled antibodies is particularly important for diagnosing diseases such as Alzheimer's where presence of specific proteins (e.g., β amyloid protein) are indicative of the disease.

A description of classes of therapeutic agents and diagnostic agents and a listing of species within each class can be found in Martindale, The Extra Pharmacopoeia, Twenty ninth Edition, The Pharmaceutical Press, London, 1989 which is incorporated herein by reference and made a part hereof. The therapeutic agents and diagnostic agents are commercially available and/or can be prepared by techniques known in the art.

In some embodiments of the present invention the enriched nanostructure composition is used to enhance the penetration of a cosmetic agent. A cosmetic agent of the present invention can be, for example, an anti-wrinkling agent, an anti-acne agent, a vitamin, a skin peel agent, a hair follicle stimulating agent or a hair follicle suppressing agent. Examples of cosmetic agents include, but are not limited to, retinoic acid and its derivatives, salicylic acid and derivatives thereof, sulfur-containing D and L amino acids and their derivatives and salts, particularly the N-acetyl derivatives, alpha-hydroxy acids, e.g., glycolic acid, and lactic acid, phytic acid, lipoic acid, collagen and many other agents which are known in the art.

The pharmaceutical agent of the present embodiments may be selected to treat or diagnose any pathology or condition. Pharmaceutical compositions of the present embodiments may be particularly advantageous to those tissues protected by physical barriers. For example, the skin is protected by an outer layer of epidermis. This is a complex structure of compact keratinized cell remnants (tough protein-based structures) separated by lipid domains. Compared to the oral or gastric mucosa, the stratum corneum is much less permeable to molecules either external or internal to the body.

Examples of skin pathologies which may be treated or diagnosed by the pharmaceutical compositions of the present embodiments include, but are not limited to acne, psoriasis; vitiligo, a keloid, a burn, a scar, a wrinkle, xerosis, ichthyosis, keratosis, keratoderma, dermatitis, pruritus, eczema, skin cancer, a hemorrhoid and a callus.

The pharmaceutical agent of the present embodiments may be selected to treat a tissue which is protected by a blood barrier (e.g. the brain). Examples of brain conditions which may be treated or diagnosed by the agents of the present embodiments include, but are not limited to brain tumor, neuropathy, Alzheimer's disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, schizophrenia, depression, manic depression, anxiety, panic disorder, social phobia, sleep disorder, attention deficit, conduct disorder, hyperactivity, personality disorder, drug abuse, infertility and head injury.

The pharmaceutical composition of the present embodiments may also comprise other physiologically acceptable carriers (i.e., in addition to the above-described enriched nanostructure composition) and excipients which will improve administration of a compound to the individual.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Pharmaceutical compositions of the present embodiments may be administered to an individual (e.g. mammal such as a human) using various routes of administration. Examples of routes of administration include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present embodiments may be manufactured by processes well known in the art, e.g., by means of conventional mixing, dissolving, granulating, dragee-making, levigating, emulsifying, encapsulating, entrapping or lyophilizing processes. Manufacturing of the nanostructures and liquid is described hereinabove.

Pharmaceutical compositions for use in accordance with the present embodiments thus may be formulated in conventional manner using the enriched nanostructure composition of the present embodiments either in the presence or absence of other physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in the enriched nanostructure composition of the present embodiments, preferably in the presence of physiologically compatible buffers such as Hank's solution, Ringer's solution, or physiological salt buffer. For transmucosal administration, other penetrants appropriate to the barrier to be permeated may be used in the formulation. Such penetrants are generally known in the art.

For oral administration, the pharmaceutical composition can be formulated readily by combining the active compounds with the enriched nanostructure composition of the present embodiments. The enriched nanostructure composition preferably enables the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present embodiments are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

For parenteral administration, the active ingredients may be combined with the enriched nanostructure composition of the present embodiments either in the presence or absence of other solvents. Aqueous injection suspensions may contain substances, which increase the viscosity of the suspension, such as sodium carboxymethyl cellulose, sorbitol or dextran. Optionally, the suspension may also contain suitable stabilizers or other agents which increase the solubility of the active ingredients to allow for the preparation of highly concentrated solutions.

The pharmaceutical compositions of the present embodiments may be formulated for topical administration. Examples of topical formulations include, but are not limited to a gel, a cream, an ointment, a paste, a lotion, a milk, a suspension, an aerosol, a spray, a foam and a serum.

Alternatively, the active ingredient may be in powder form for constitution with the enriched nanostructure composition of the present embodiments, before use.

The pharmaceutical composition of the present embodiments may also be formulated in rectal compositions such as suppositories or retention enemas, using, e.g., conventional suppository bases such as cocoa butter or other glycerides.

Pharmaceutical compositions suitable for use in context of the present embodiments include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the embodiments, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The present embodiments further comprise invention a novel compositions which can enhance both cell growth and cell fusion. Specifically, the present invention can be used to enhance monoclonal antibody production.

The production of human monoclonal antibodies requires the immortalization of human B-lymphocytes by fusion with a partner cell-line of a myeloid source. However, since the only human B-cells that are available for monoclonal antibody production are the ones that circulate in the peripheral blood, the source of cells for monoclonal antibody production is limited.

In addition, it has proven difficult to produce high levels of isolated monoclonal antibodies from a hybridoma cell culture as the quantities of secreted monoclonal antibodies are typically not high.

In order to bridge the theoretical and the practical outcomes of monoclonal antibody production, the efficiency of the fusion process needs to be very high, to overcome the rarity of the B-cells obtained from peripheral blood, thus making their chances of immortalization higher. In addition methods need to be sought to enhance both the stability of hybridomas and secretion of monoclonal antibodies therefrom.

In some embodiments of the present invention the enriched nanostructure composition promote both cell fusion and cell stability.

In some embodiments of the present invention the enriched nanostructure composition promote fusion of human peripheral blood mononuclear cells (PBMC) and fusion partner (MFP-2) cells and also promotes the stability of the hybridomas produced therefrom. In some embodiments of the present invention the enriched nanostructure composition increases antibody secretion from the hybridomas. Thus, the enriched nanostructure composition of the present embodiments may aid in the isolation and production of monoclonal antibodies.

The present embodiments exploits this finding to provide novel compositions that promote not only monoclonal antibody production, but also enhance fusion between other eukaryotic cells as well as to enhance growth of cells in general and mesenchymal stem cells in particular.

Thus, according to an aspect of the present embodiments there is provided a method of cell-fusion, the method comprising fusing cells in a medium comprising the enriched nanostructure composition of the present embodiments, thereby affecting cell-fusion.

As used herein the phrase “cell-fusion” refers to the merging, (either ex vivo or in vivo) of two or more viable cells.

Cell-fusion may be accomplished by any method of combining cells under fusogenic conditions. For example cells may be fused in the presence of a fusion stimulus such as polyethylene glycol (PEG) or Sendai virus (See, for example, Harlow & Lane (1988) in Antibodies, Cold Spring Harbor Press, New York). Alternatively, cells may be fused under appropriate electrical conditions.

Without being bound to theory, it is believed that the long-range interactions between the nanostructures lends to the unique characteristics of the enriched nanostructure composition. One such characteristic is that the enriched nanostructure composition of the present embodiments is able to enhance the fusion process between two cell types.

In some embodiments of the present invention the enriched nanostructure composition can aid in the process of cell-fusion. Examples of cells include primary cells and immortalized cells, identical cells and non-identical cells, human cells and non-human cells.

The phrase “immortalized cells” refers to cells or cell lines that can be passaged in cell culture for several generations or indefinitely. An example of an immortalized cell is a tumor cell.

Thus, for example, the enriched nanostructure composition of the present embodiments may be used to assist in the ex vivo fusion between tumor cells and antibody producing cells (e.g. B lymphocytes) to produce a hybridoma.

The term “hybridoma” as used herein refers to a cell that is created by fusing two cells, a secreting cell from the immune system, such as a B-cell, and an immortal cell, such as a myeloma, within a single membrane. The resulting hybrid cell can be cloned, producing identical daughter cells. Each of these daughter clones can secrete cellular products of the immune cell over several generations.

In some embodiments of the present invention, the B lymphocytes are human B lymphocytes. In some embodiments of the present invention, the B lymphocytes are those which circulate in the peripheral blood e.g. PBMCs.

Examples of tumor cells which may be used to produce hybridomas according to the present embodiments include mouse myeloma cells and cell lines, rat myeloma cell lines and human myeloma cell lines.

Preferably, the myeloma cell lines comprise a marker so a selection procedure may be established. For example the myeloma cell lines may be HGPRT negative (Hypoxanthine-guanine phosphoribosyl transferase) negative. Specific examples thereof include: X63-Ag8 (X63), NS1-Ag4/1(NS-1), P3X63-Ag8.UI (P3UI), X63-Ag8.653 (X63.653), SP2/0-Ag14 (SP2/0), MPC11-45.6TG1.7 (45.6TG), FO, S149/5XXO.BU.1, which are derived from mice; 210.RSY3.Ag.1.2.3 (Y3) derived from rats; and U266AR (SKO-007), GM1500 GTG-A12 (GM1500), UC729-6, LICR-LOW-HMy2 (HMy2), 8226AR/NIP4-1 (NP41) and MFP-2, which are derived from humans.

According to the present embodiments, the tumor cells and/or B lymphocytes are incubated in a medium (e.g. a culture medium) comprising the enriched nanostructure composition of the present embodiments.

As used herein the phrase “culture medium” refers to a medium having a composition which allows eukaryotic cells to remain viable for at least 12 hours and preferably to replicate.

Incubation in the enriched nanostructure composition of the present embodiments may be effected prior to during and/or following the fusion procedure in order to increase the number of hybridomas. Incubation in the enriched nanostructure composition of the present embodiments prior to the fusion process may be effected for any length of time so as to enhance hybridoma generation. Preferably, incubation is for more than one day.

The liquid portion of a culturing medium may be wholly or partly exchanged for the enriched nanostructure composition of the present composition as further described hereinbelow.

The culture medium, according to some embodiments of the present invention is typically selected on an empirical basis since each cell responds to a different culture medium in a particular way. Examples of culture medium are further described hereinbelow.

The enriched nanostructure composition of the present embodiments may be used to aid in the ex-vivo fusion between other cells such as tumor cells and dendritic cells. It has been shown that such fused cells may be effective as anti-cancer vaccines [Zhang K et al., World J Gastroenterol. 2006 Jun. 7; 12(21):3438-41].

The enriched nanostructure composition of the present embodiments may be used to aid in the in vivo fusion between somatic cells and stem cells. Because of their powerful generative and regenerative abilities, stem cells may be used to repair damage in the bone marrow and to different organs such as the liver, brain and heart. It has been shown that some of the stem cells' repair properties come from their ability to fuse with cells that are naturally resident in the organs they are repairing [Wang et al., 2003, Nature 422, 897-901]. Accordingly, the enriched nanostructure composition of the present embodiments may be used to enhance fusion between stem cells and somatic cells such as bone cells and muscle cells. Thus, stem cells may be treated with the enriched nanostructure composition of the present embodiments so that they fuse quicker and more efficiently to a target site, thereby directing the stem-cell repair process.

The enriched nanostructure composition of the present embodiments may also be used for in vivo transferring nucleic acids by way of cell-fusion. See e.g., Hoppe U C, Circ Res. 1999 Apr. 30; 84(8):964-72

Another ex vivo fusion process which may be aided by the composition of the present embodiments is the fusion between embryonic stem cells and human cells. Such fusions were shown to generate hybrids which behaved in a similar manner to embryonic stem cells, thus generating genetically matched stem cells for transplants. Specifically, human embryonic stem (hES) cells were fused with human fibroblasts, resulting in hybrid cells that maintain a stable tetraploid DNA content and have morphology, growth rate, and antigen expression patterns characteristic of hES cells [Cowan et al., Science, 2005 Aug. 26; 309(5739):1369-73].

Yet another ex vivo fusion process which may be facilitated by the composition of the present embodiments is somatic cell nuclear transfer. This is the process by which a somatic cell is fused with an enucleated oocyte. The nucleus of the somatic cell provides the genetic information, while the oocyte provides the nutrients and other energy-producing materials that are necessary for development of an embryo. This procedure is used for cloning and generation of embryonic stem cells.

The enriched nanostructure composition of the present embodiments can enhance the whole process of monoclonal antibody production including the fusion process, the cloning of hybridomas generated thereby and the secretion of antibodies therefrom. It is expected that cloned hybridomas generated in the presence of the enriched nanostructure composition of the present embodiments will be more stable than cloned hybridomas generated in the absence of the enriched nanostructure composition of the present embodiments.

Thus, according to an aspect of some embodiments of the present invention, there is provided a method of generating a monoclonal antibody, the method comprising fusing an immortalizing cell with an antibody producing cell to obtain a hybridoma in a medium comprising the enriched nanostructure composition.

As used herein, the phrase “monoclonal antibody” refers to an immune molecule that comprises a single binding affinity for any antigen with which it immunoreacts.

According to the present embodiments, monoclonal antibodies are generated by fusing an immortalizing cell with an antibody producing cell to produce hybridomas in the enriched nanostructure composition of the present embodiments as described hereinabove. The generated hybridomas may then be cloned. According to some embodiments of the present invention, the cloning is effected by incubating single hybridomas in a medium comprising the enriched nanostructure composition of the present embodiments.

Since cloned hybridomas generated in the presence of the enriched nanostructure composition of the present embodiments are more stable than those generated in the absence thereof, the cloning procedure typically does not require the addition of a stabilizing factor such as HCF.

Following generation of hybridomas and optional cloning thereof, monoclonal antibodies may be screened and harvested. Many methods of screening are known in the art including functional and structural assays. An exemplary method for screening hybridomas is described in Example 2 hereinbelow using a sandwich ELISA assay.

Techniques for harvesting of monoclonal antibodies are also well known in the art and typically comprise standard protein purification methods.

According to an aspect of some embodiments of the present invention, there is provided an article-of-manufacture, which comprises the composition of the present embodiments as described hereinabove, being packaged in a packaging material and identified in print, in or on the packaging material for use in generation of monoclonal antibodies, as described herein.

Since the composition of the present embodiments can enhance stabilization of eukaryotic cellular matter such as the hybridomas described hereinabove, the present inventors have realized that the composition of the present embodiments may be exploited to enhance stabilization of other eukaryotic cellular matter.

Thus, according an aspect of some embodiments of the present invention there is provided a method of culturing eukaryotic cells. The method comprises incubating the cells in a medium comprising the enriched nanostructure composition of the present embodiments.

Without being bound to theory, the present inventors believe that the enriched nanostructure composition of the present embodiments is particularly appropriate for use in a culture medium for a number of reasons.

Firstly, in some embodiments of the present invention the enriched nanostructure composition is capable of increasing the proliferation rate of cells cultured therein.

Secondly, in some embodiments of the present invention the enriched nanostructure composition enhances the solubility of agents. This may be particularly relevant for enhancing the solubility of a water-insoluble agent that needs to be added to a culture medium.

Thirdly, in some embodiments of the present invention the enriched nanostructure composition comprises an enhanced buffering capacity i.e. comprises a buffering capacity greater than water. This may be relevant for cells that are particularly pH sensitive.

Fourthly, in some embodiments of the present invention the enriched nanostructure composition is capable of stabilizing proteins. This may be particularly relevant if a non-stable peptide agent needs to be added to a culture medium or for stabilizing a cell secreted peptide agent.

It should be appreciated that according to the present embodiments, the cells may be cultured for any purposes, such as, but not limited to for growth, maintenance and/or for cloning. In addition, it should be appreciated that the incubation time is not restricted in any way and cells may be cultured in the composition of the present embodiments for as long as required.

The composition of the present embodiments may be particularly useful for culturing cells which require autocrine secretion of factors which are typically present at low concentrations. For example, mesenchymal stem cells were shown to secrete DKK1, which enhances proliferation. The ordered structure of the composition of the present embodiments may serve to effectively increase the DKK1 concentration thereby enhancing its growth.

The composition of the present embodiments may also be particularly useful for culturing cells which have a tendency to be non-stable. Examples of such cells include, but are not limited to hybridomas, cells which are being re-cultured following freezing and cells which are present at low concentrations.

The present inventors contemplate exchanging all or a part of the water content of any eukaryotic cell culture medium with the enriched nanostructure composition of the present embodiments. Removal of the water content of the medium may be effected using techniques such as lyophilization, air-drying and oven-drying. Thus, the liquid portion of the culturing medium may comprise 5%, more preferably 10%, more preferably 20%, more preferably 40%, more preferably 60%, more preferably 80% and even more preferably 100% of the enriched nanostructure composition of the present embodiments.

Many media are also commercially available as dried components. As such, the enriched nanostructure composition of the present embodiments may be added without the prior need to remove the water component of the media.

Examples of eukaryotic cell culture media include DMEM, RPMI, Ames Media, CHO cell media, Ham's F-10 medium, Ham's F-12 medium, Leibovita L-15 medium, McCoy's medium, MEM Alpha Medium. Such media are widely available from Companies such as Sigma Aldrich and Invitrogen.

It will be appreciated that the medium may comprise other components such as growth factors, serum and antibiotics. Such components are commercially available e.g. from Sigma Aldrich and Invitrogen.

Preferably the enriched nanostructure composition of the present embodiments is sterilized (e.g. by filter sterilization) prior to incubating the cells therein.

According to an aspect of the some embodiments of the present invention, there is provided an article-of-manufacture, which comprises the composition of the present embodiments as described hereinabove, being packaged in a packaging material and identified in print, in or on the packaging material for culturing eukaryotic cells, as described herein.

As mentioned hereinabove, the composition of the present embodiments may be manufactured as a ready-made culture medium. Accordingly, there is provided a cell culture medium comprising a eukaryotic cell culture medium and the enriched nanostructure composition of the present embodiments.

According to an aspect of some embodiments of the present invention there is provided a method of dissolving or dispersing cephalosporin, comprising contacting the cephalosporin with nanostructures and liquid under conditions which allow dispersion or dissolving of the substance, wherein the nanostructures comprise a core material of a nanometric size enveloped by ordered fluid molecules of the liquid, the core material and the envelope of ordered fluid molecules being in a steady physical state.

The cephalosporin may be dissolved in a solvent prior or following addition of the enriched nanostructure composition of the present embodiments in order to aid in the solubilizing process. It will be appreciated that the present embodiments contemplates the use of any solvent including polar, non-polar, organic, (such as ethanol or acetone) or non-organic to further increase the solubility of the substance.

The solvent may be removed (completely or partially) at any time during the solubilizing process so that the substance remains dissolved/dispersed in the enriched nanostructure composition of the present embodiments. Methods of removing solvents are known in the art such as evaporation (i.e. by heating or applying pressure) or any other method.

The present embodiments further comprise kits and articles of manufacture which can be used to enhance the detection of an analyte.

The medical and diagnostic testing industries are constantly searching for more sensitive methods for detecting biomolecules. For example, medicine has an obvious need for highly sensitive methods of detecting viruses. More sensitive assays for the detection of chemicals or other substances would also be of use in a broad range of environmental areas, where early detection could trigger corrective action early enough to head off disaster. A highly sensitive detection technology could also be useful for the optimized control of semiconductor fabrication.

In some embodiments of the present invention the enriched nanostructure composition enhances detection of an analyte. In various exemplary embodiments of the invention the enriched nanostructure composition increases the sensitivity of an ECL protein detecting system.

Thus, according to an aspect of some embodiments of present invention there is provided an article of manufacture comprising packaging material and a composition identified for enhancing detection of a detectable moiety being contained within the packaging material, the enriched nanostructure composition being the enriched nanostructure composition described herein.

Without being bound to theory, it is believed that the long-range interactions between the nanostructures lends to the unique characteristics of the enriched nanostructure composition such that it enhances the sensitivity of a detection system. For example, the enriched nanostructure composition of the present embodiments can shield and stabilize proteins from the effects of heat and/or comprise an enhanced buffering capacity. Both these factors may contribute to the state of proteins in the detection system, enhancing the overall sensitivity of the detection system.

The ability of the enriched nanostructure composition of the present embodiments to enhance the solubility of agents can lead to an enhanced sensitivity of the detection system.

It will be appreciated that the enriched nanostructure composition of the present embodiments described hereinabove can form a part of a kit.

Thus, according to an aspect of some embodiments of the present invention there is provided a kit for detecting an analyte comprising (i) a detectable agent; and (ii) the enriched nanostructure composition of the present embodiments.

The kits of the present embodiments may, if desired, be presented in a pack which may contain one or more units of the kit of the present embodiments. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

As used herein, the term “analyte” refers to a molecule or compound to be detected. Suitable analytes include organic and inorganic molecules, including biomolecules. The analyte may be an environmental or clinical chemical or pollutant or biomolecule, including, but not limited to, pesticides, insecticides, toxins, therapeutic and abused drugs, hormones, antibiotics, organic materials, and solvents. Suitable biomolecules include, but are not limited to, polypeptides, polynucleotides, lipids, carbohydrates, steroids, whole cells [including prokaryotic (such as pathogenic bacteria) and eukaryotic cells, including mammalian tumor cells], viruses, spores, etc. Particularly preferred analytes are proteins including enzymes; drugs, antibodies; antigens; cellular membrane antigens and receptors (neural, hormonal, nutrient, and cell surface receptors) or their ligands.

The detection kits of the present embodiments show enhanced sensitivity by virtue of a liquid composition comprising liquid and nanostructures.

The present embodiments envisages solubilizing at least one component required for detection in the composition comprising liquid and nanostructures and/or performing the detection assay, wherein the water component is at least partly exchanged for the composition comprising liquid and nanostructures. The liquid portion of the detection assay may comprise 5%, more preferably 10%, more preferably 20%, more preferably 40%, more preferably 60%, more preferably 80% and even more preferably 100% of the enriched nanostructure composition of the present embodiments.

As well as comprising a composition comprising liquid and nanostructures, the kits of the present embodiments also comprise a detectable agent.

According to some embodiments of the present invention, the detectable agent is directly detectable typically by virtue of its emission of radiation of a particular wavelength (e.g. a fluorescent agent, phosphorescent agent or a chemiluminescent agent).

In order to detect a specific analyte, typically such detectable agents comprise affinity recognition moieties which bind to the target analyte. Examples of affinity recognition moieties include, but are not limited to avidin derivatives (e.g. avidin, strepavidin and neutravidin), antibodies and polynucleotides.

Avidin is a highly cationic 66,000-dalton glycoprotein with an isoelectric point of about 10.5. Streptavidin is a nonglycosylated 52,800-dalton protein with a near-neutral isoelectric point. Neutravidin is a deglycosylated form of avidin. All of these proteins have a very high affinity and selectivity for biotin, each capable of binding four biotins per molecule. A detectable agent comprising an avidin recognition moiety may be used for detecting naturally occurring biotinylated biomolecules, or biomolecules that have been artificially manipulated to comprise biotin.

The term “antibody” as used in this embodiments includes intact molecules as well as functional fragments thereof, such as Fab, F(ab′)2, and Fv that are capable of binding to specific proteins or polypeptides.

The term “polynucleotide” as used herein, refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions. Labeled polynucleotides may be used to detect polynucleotides in a sample that are capable of hybridizing thereto.

As used herein, the phrase “capable of hybridizing” refers to base-pairing, where at least one strand of the nucleic acid agent is at least partly homologous to H19 mRNA.

According to some embodiments of the present invention, the detectable agent of the kit of the present embodiments may also be non-directly detectable. For example, the detectable agent may be a substrate for an enzymatic reaction which is capable of generating a detectable product.

Substrates capable of generating a fluorescent product typically comprise fluorophores. Such fluorophores may be derived from many molecules including but not limited to coumarin, fluorescein, rhodamine, resorufine and DDAO.

Examples of substrates which are capable of generating a fluorescent product include, but are not limited to substrates yielding soluble fluorescent products (e.g. substrates derived from water-soluble coumarins, substrates derived from water-soluble green to yellow fluorophores, substrates derived from water-soluble red fluorophores, thiol-reactive fluorogenic substrates, lipophilic fluorophores, pentafluorobenzoyl fluorogenic enzyme substrate); substrates yielding insoluble fluorescent products, substrates based on excited-state energy transfer and fluorescent derivatization reagents for discontinuous enzyme assays). Details regarding such substrates may be found on the Invitrogen website (e.g. www.probes.invitrogen.com/handbook/sections/1001.html).

Specific examples of substrates capable of generating a fluorescent product include, but are not limited to fluorescein di-β-D-galactopyranoside (FDG), resorufine β-D-galactopyranoside, DDAO galactoside, β-methylumbelliferyl β-D-galactopyranoside, 6,8-Difluoro-4-methylumbelliferyl β-D-galactopyranoside, 3-carboxyumbelliferyl-β-D-galactopyranoside, ELF 97 phosphate, 5-chloromethylfluorescein di-β-D-galactopyranoside (CMFDG), 4-methylumbelliferyl-3-D-glucuronide, Fluorescein di-β-D-glucuronide, PFB Aminofluorescein Diglucuronide, ELF 97-β-D-glucuronide, BODIPY FL chloramphenicol Substrate™, and 10-acetyl-3,7-dihydroxyphenoxazine.

Examples of substrates capable of generating a chemiluminescent product include, but are not limited to luciferin, luminol, isoluminol, acridane, phenyl-10-methylacridane-9-carboxylate, 2,4,6-trichlorophenyl-1-0-methylacridane-9-carboxylate, pyrogallol, phloroglucinol and resorcinol.

Examples of substrates capable of generating a chromogenic product include, but are not limited to BCIP, 5-bromo-4-chloro-3-indolyl-β-D-glucuronic acid (X-GlcU) and 5-bromo-6-chloro-3-indolyl-β-D-glucuronide, 5-bromo-4-chloro-3-indolyl-β-D-galactopyranoside (X-Gal), diaminobenzidine (DAB), Tetramethylbenzidine (TMB) and o-Phenylenediamine (OPD).

The kits may be useful in a variety of detection assays.

Following is a list of assays for the detection of polynucleotides, which may be effected using the kits of the present embodiments.

### Northern Blot Analysis

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using enzyme linked nucleotides. Detection may be effected using colorimetric reaction or chemiluminescence. This method allows both quantitation of an amount of particular RNA molecules and determination of its identity by a relative position on the membrane which is indicative of a migration distance in the gel during electrophoresis.

### RNA In Situ Hybridization Stain

In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using chemiluminescence associated probes or to a colorimetric reaction which reveals signals generated using enzyme-linked labeled probes.

### Oligonucleotide Microarray

In this method oligonucleotide probes capable of specifically hybridizing with the polynucleotides of the present embodiments are attached to a solid surface (e.g., a glass wafer). Each oligonucleotide probe is of approximately 20-25 nucleic acids in length. To detect the expression pattern of the polynucleotides of the present embodiments in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetrix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Following is a list of assays for the detection of polypeptides, which may be effected using the kits of the present embodiments.

### Western Blot

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

### Fluorescence Activated Cell Sorting (FACS)

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Immunohistochemical Analysis

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

### In Situ Activity Assay

According to this method, a chromogenic substrate is applied on the cells containing an active enzyme and the enzyme catalyzes a reaction in which the substrate is decomposed to produce a chromogenic product visible by a light or a fluorescent microscope.

According to some embodiments of the present invention, the kits may be used to detect immobilized polypeptides or polynucleotides using a chemiluminescent detection assay.

In this assay, the target analyte is bound either directly or indirectly to an enzyme (e.g. horseradish peroxidase) which in the presence of an oxidizing agent is capable of catalyzing the oxidation of chemiluminescent substrates. Following oxidation the substrates are in an excited state and emit detectable light waves. Strong enhancement of the light emission may be produced by enhancers.

Accordingly, such kits may comprise, in addition to the enriched nanostructure composition of the present embodiments and the detectable agent (i.e. chemiluminescent compounds such as luminol and those described hereinabove) enzymes capable of oxidizing the chemiluminescent substrates. Typically the enzyme is conjugated to an antibody or an avidin derivative such as strepavidin. Examples of such enzymes include, but are not limited to horseradish peroxidase, glucose oxidase, cholesterol oxidase and catalase.

The kits according to the present embodiments may also comprise an oxidant. Exemplary oxidizing agents include hydrogen peroxide, urea hydrogen peroxide, sodium carbonate hydrogen peroxide or a perborate salt. Other oxidants or oxidizing agents known to those skilled in the art may be used herein. The preferred oxidant is either hydrogen peroxide or urea hydrogen peroxide and mixtures thereof.

As noted above, the kits of the present embodiments may, also, include a chemiluminescence enhancer. Generally, the enhancer used herein comprises an organic compound which is soluble in an organic solvent or in a buffer and which enhances the luminescent reaction between the chemiluminescent organic compound, the oxidant and the enzyme or other biological molecule. Suitable enhancers include, for example, halogenated phenols, such as p-iodophenol, p-bromophenol, p-chlorophenol, 4-bromo-2-chlorophenol, 3,4-dichlorophenol, alkylated phenols, such as 4-methylphenol and, 4-tert-butylphenol, 3-(4-hydroxyphenyl)propionate and the like, 4-benzylphenol, 4-(2′,4′-dinitrostyryl)phenol, 2,4-dichlorophenol, p-hydroxycinnamic acid, p-fluorocinnamic acid, p-nitrocinnamic acid, p-aminocinnamic acid, m-hydroxycinnamic acid, o-hydroxycinnamic acid, 4-phenoxyphenol, 4-(4-hydroxyphenoxy)phenol, p-phenylphenol, 2-chloro-4-phenylphenol, 4′-(4′-hydroxyphenyl)benzophenone, 4-(phenylazo) phenol, 4-(2′-carboxyphenylaza)phenol, 1,6-dibromonaphtho-2-ol, 1-bromonaphtho-2-ol, 2-naphthol, 6-bromonaphth-2-ol, 6-hydroxybenzothiazole, 2-amino-6-hydroxybenzothiazole, 2,6-dihydroxybenzothiazole, 2-cyano-6-hydroxybenzothiazole, dehydroluciferin, firefly luciferin, phenolindophenol, 2,6-dichlorophenolindophenol, 2,6-dichlorophenol-o-cresol, phenolindoaniline, N-alkylphenoxazine or substituted N-alkylphenoxazine, N-alkylphenothiazine or substituted N-alkylphenothiazine, N-alkylpyrimidylphenoxazine or substituted N-alkylpyrimidylphenoxazine, N-alkylpyridylphenoxazine, 2-hydroxy-9-fluorenone or substituted 2-hydroxy-9-fluorenone, 6-hydroxybenzoxazole or substituted 6-hydroxybenzoxazole. Still other useful compounds include a protected enhancer that can be cleaved by the enzyme such as p-phenylphenol phosphate or p-iodophenol phosphate or other phenolic phosphates having other enzyme cleavable groups, as well as p-phenylene diamine and tetramethyl benzidine. Other useful enhancers include fluorescein, such as 5-(n-tetradecanyl)amino fluorescein and the like.

According to an aspect of some embodiments of the present invention, the kits may be used to detect immobilized polypeptides or polynucleotides using a fluorescent or chromogenic detection assay. Instead of comprising horseradish peroxidase or a derivate thereof, such kits typically comprise alkaline phosphatase and a fluorescent or chromogenic substrate. Oxidising agents for the production of chromogenic products may also be included in the kits such as potassium ferricyanide and Nitro blue tetrazolium (NBT).

The kits of the present embodiments may also be used for detecting the expression of several common reporter genes in cells and cell extracts. Thus the kits may comprise substrates for β-galactosidase β-glucuronidase, secreted alkaline phosphatase, chloramphenicol acetyltransferase and luciferase.

The kits of the present embodiments may further include inhibitors for the enzymatic reactions. Examples of such inhibitors include, but are not limited to levamisole, L-p-bromotetramisole, tetramisole and 5,6-Dihydro-6-(2-naphthyl)imidazo-[2,1-b]thiazole.

According to an aspect of some embodiments of the present invention there is provided a method of dissolving or dispersing cephalosporin, comprising contacting the cephalosporin with nanostructures and liquid under conditions which allow dispersion or dissolving of the substance, wherein the nanostructures comprise a core material of a nanometric size enveloped by ordered fluid molecules of the liquid, the core material and the envelope of ordered fluid molecules being in a steady physical state.

The cephalosporin may be dissolved in a solvent prior or following addition of the enriched nanostructure composition of the present embodiments in order to aid in the solubilizing process. It will be appreciated that the present embodiments contemplates the use of any solvent including polar, non-polar, organic, (such as ethanol or acetone) or non-organic to further increase the solubility of the substance.

The solvent may be removed (completely or partially) at any time during the solubilizing process so that the substance remains dissolved/dispersed in the enriched nanostructure composition of the present embodiments. Methods of removing solvents are known in the art such as evaporation (i.e. by heating or applying pressure) or any other method.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Examples

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Carbon Measurements

Carbon dioxide enriched compositions containing water, nanostructures and CO₂ phase prepared according to various exemplary embodiments of the present invention were subjected to total carbon (TC), total organic carbon (TOC) and inorganic carbon (IC) measurements. Similar measurements were perfumed for RO water. All measurements were performed using a Sievers Carbon Analyzer.

Table 1 summarizes the results of TOC, IC and TC=TOC+IC, for RO water and the enriched nanostructure composition of the present embodiments. The enriched nanostructure composition of the present embodiments is interchangeable referred to in this example as “gas enriched nanostructure composition” abbreviated “GENC.”

TABLE 1

| sample | | | | | | | | |
|---|---|---|---|---|---|---|---|---|
| type | vial No. | Rep | TOC | | IC | | TC | |
| RO water | 1 | 1 | 96.4 | ppb | 168 | ppb | 264.4 | ppb |
| | | 2 | 85.5 | ppb | 175 | ppb | 260.5 | ppb |
| | | 3 | 83.1 | ppb | 178 | ppb | 261.1 | ppb |
| | | 4 | 83.6 | ppb | 180 | ppb | 263.6 | ppb |
| | | Average | 84.1 | ppb | 178 | ppb | 261.7 | ppb |
| | | SD | 1.27 | ppb | 2.92 | ppb | 1.64 | ppb |
| | | RSD | 1.51% | | 1.42% | | 0.63% | |
| | 2 | 1 | 90.7 | ppb | 139 | ppb | 229.7 | ppb |
| | | 2 | 87.7 | ppb | 141 | ppb | 228.7 | ppb |
| | | 3 | 88.5 | ppb | 142 | ppb | 230.5 | ppb |
| | | 4 | 90.7 | ppb | 144 | ppb | 234.7 | ppb |
| | | Average | 89.0 | ppb | 142 | ppb | 231.3 | ppb |
| | | SD | 1.55 | ppb | 1.53 | ppb | 3.08 | ppb |
| | | RSD | 1.75% | | 1.07% | | 1.33% | |
| GENC | 3 | 1 | 261 | ppb | 1.21 | ppm | 1.471 | ppm |
| | | 2 | 284 | ppb | 1.22 | ppm | 1.504 | ppm |
| | | 3 | 277 | ppb | 1.23 | ppm | 1.507 | ppm |
| | | 4 | 278 | ppb | 1.24 | ppm | 1.518 | ppm |
| | | Average | 280 | ppb | 1.23 | ppm | 1.510 | ppm |
| | | SD | 3.79 | ppb | 10.0 | ppm | 7.37 | ppb |
| | | RSD | 1.35% | | 0.81% | | 0.49% | |
| | 4 | 1 | 178 | ppb | 250 | ppb | 428 | ppb |
| | | 2 | 169 | ppb | 246 | ppb | 415 | ppb |
| | | 3 | 167 | ppb | 243 | ppb | 410 | ppb |
| | | 4 | 168 | ppb | 243 | ppb | 411 | ppb |
| | | Average | 168 | ppb | 244 | ppb | 412 | ppb |
| | | SD | 1.00 | ppb | 1.73 | ppb | 2.65 | ppb |

| | | | |
|---|---|---|---|
| RSD | 0.60% | 0.71% | 0.64% |

Table 1 demonstrates that the enriched nanostructure composition of the present embodiments contains larger amount of carbon as compared to RO water. For RO water, the average TC values were 261.7 ppb (vial No. 1) and 231.3 ppb (vial No. 2). For ENPD liquid, the average TC values were 1.51 ppm (vial No. 3) and 412 ppb (vial No. 4). Thus, the average TC values for GENC is from about 1.5 times to about 6.5 times higher than the average TC values for RO water.

### Example 2

### Effect of Initial Carbon Content

Various batches of enriched nanostructure composition were prepared according to various exemplary embodiments of the present invention. The carbon content was measured immediately following the production of each batch. Additional measurements included electrochemical deposition (ECD), ζ potential, pH and conductivity.

The enriched nanostructure composition of the present embodiments is interchangeable referred to in this example as “gas enriched nanostructure composition” abbreviated “GENC.”

### Methods

### Preparation of GENC

GENC was prepared using system 140 (see FIG. 4). Solid powder was Barium titanate (BT) or Hydroxyapatite (HA). The liquid was water for injection (WFI) grade. The gas was CO₂, at least 99.9% pure, which was introduced either directly to the water or to the sleeve 148.

The reservoir 154 was filled with 10 L of WFI which was brought to a temperature of about 2° C. About 0.45 g of solid powder was placed in the furnace 142 which was heated to a temperature of about 850° C.

### ζ Potential

ζ potential was measured using a ZetaSizer (model ZEN3600, Malvern Instruments, UK).

### Electrochemical Deposition

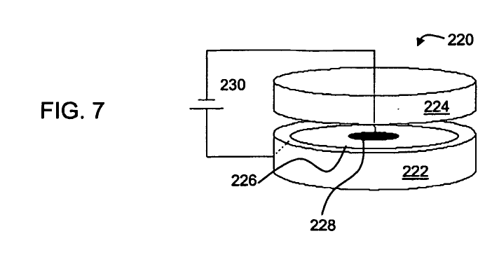

The experimental setup is illustrated in FIG. 7. A quasi-two-dimensional cell 220, 125 mm in diameter, included a Plexiglas base 222 and a Plexiglas cover 224. When cover 224 was positioned on base 222 a quasi-two-dimensional cavity, about 1 mm in height, was formed. Two concentric electrodes 226 (external) and 228 (internal) were positioned in cell 220 and connected to a voltage source 230 of 12.4±0.1 V. External electrode 226 was shaped as a ring, 90 mm in diameter, and made of a 0.5 mm copper wire. Internal electrode 228 was shaped as a disc having a thickness of 0.1 mm and diameter of 28 mm. The external electrode was connected to the positive pole of the voltage source and the internal electrode was connected to the negative pole thereof.

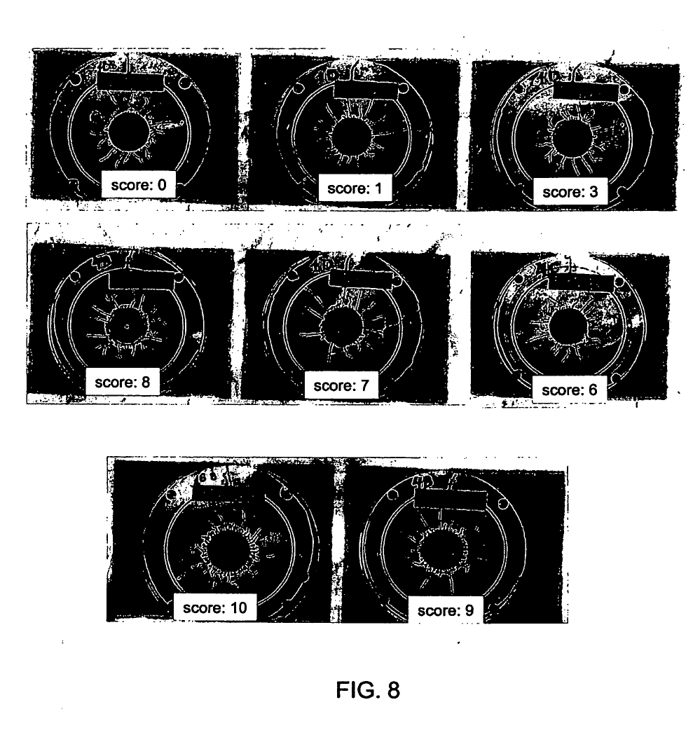

The obtained ECD patterns were scored on a 0 to 10 scale. Typically, eight integer value numbers were used as scores: 0, 1, 3, 6, 7, 8, 9 and 10. These scores are shown in the representative images of FIG. 8. Scores 0 and 1 was declared as a “negative” results, and scores above 8, 9 and 10 were declared “positive” results.

The following protocol was employed:

(a) Testing Solution Preparation
in a 500 ml volumetric flask, prepare a 0.2M ZnSO₄ solution by weighing 28.75 g of ZnSO₄ (MW=287.5) in RO water and fill to volume

(b) System Suitability
(i) thoroughly wash the ECD cell with RO water:
(ii) pour about 12 ml of 0.2M ZnSO₄ solution on the base
(iii) place the internal electrode in the middle of the base
(iv) lay the cover on top of the base, avoiding air bubbles, and put a weight on top of the cover
(v) wait about 10 minutes
(vi) activate the voltage source
(vii) repeat (a)(i) through (a)(vi) until a negative ECD pattern is developed

(c) ECD Test
(i) once a negative pattern has been developed, wash the ECD cell with RO water
(ii) evenly spread about 12 ml of GENC on both cover and base of the ECD cell, wait about 30 minutes and drain the GENC from the cover and base
(iii) pour RO water on the cover and base, wait 5 minutes and drain, repeat this phase once
(iv) pour 0.2M ZnSO₄ solution on the base, and lay the cover on top of the base, avoiding air bubbles
(v) wait about 30 minutes
(vi) activate the voltage source
(vii) observe and score the developed ECD pattern

### pH Test

pH measurements were performed using bromothymol bleu (pH 6.0-7.6) and phenol red (pH 6.8-8.4), both in ethanolic solutions according to the USP monograph

### Conductivity

Conductivity indicators were 1413 μS, 111.8 μS and 12.88 μS. The measurement was performed after checking the calibration. Before measurement, sample of GENC is taken into a class-A vial and the probe washed with ultra pure water.

### Results

Table 2 below lists ECD scores, IC contents in ppm, pH values, conductivity in μS and ζ potential for some samples of the CO₂ enriched nanostructure composition of the present embodiments. Further listed in Table 2 are the solid powders which were used in the preparation of the respective samples, and which form the core of the nanostructures of the enriched nanostructure composition. The core materials were barium titanate (BT) and hydroxyapatite (HA).

Some samples were not subjected to all tests, and the respective entry for these samples is blank. For sample No. 15, the type of solid powder was not recorded and the “core” entry for sample No. 15 is blank.

Table 2 demonstrates that the pH, conductivity and ζ potential generally rise with the IC content. Table 2 further demonstrates that higher IC content generally results in higher ECD scores.

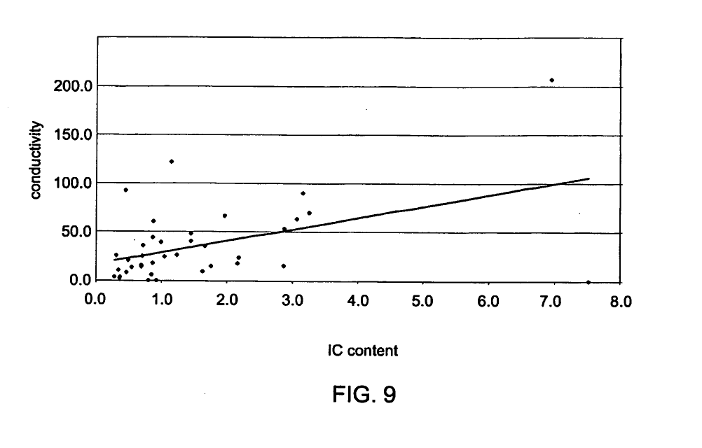

FIG. 9 is a graph showing the conductivity as a function of the IC content. Also shown is a linear regression line demonstrating that the conductivity rises with the IC content.

TABLE 2

| Sample No. | Core | ECD | Initial IC | pH | Conductivity | ζ |
|---|---|---|---|---|---|---|
| 1 | BT | 7 | 0.497 ± 0.10 | 6.61 ± 0.16 | 20.50 ± 12.30 | −7.09 |
| 2 | BT | 8 | 6.950 ± 1.89 | 7.56 ± 0.26 | 207.06 ± 19. | −5.29 |
| 3 | BT | 8 | 2.863 ± 0.09 | 6.58 ± 0.79 | 15.15 ± 1.17 | −35.87 |
| 4 | BT | 6 | 1.962 ± 0.53 | 7.33 ± 0.38 | 65.98 ± 36.55 | −6.67 |
| 5 | BT | 8 | 0.711 ± 0.16 | 6.83 ± 0.30 | 24.58 ± 7.38 | |
| 6 | BT | 10 | 0.450 ± 0.53 | 7.04 ± 0.04 | 92.18 ± 5.09 | |
| 7 | BT | 8 | 1.452 ± 0.37 | 7.05 ± 0.07 | 47.95 ± 5.73 | −12.40 |
| 8 | BT | 10 | 0.863 ± 0.15 | 6.80 ± 0.14 | 43.60 ± 2.83 | |
| 9 | BT | 10 | 0.696 ± 0.05 | 5.75 ± 0.64 | 15.45 ± 2.33 | |
| 10 | BT | 9 | 2.162 ± 0.04 | 6.54 ± 0.25 | 17.76 ± 5.19 | −22.80 |
| 11 | BT | 6 | 0.697 ± 0.02 | 6.64 ± 0.34 | 14.17 ± 1.99 | −4.44 |
| 12 | BT | 9 | 1.630 ± 0.03 | 6.37 ± 0.20 | 9.23 ± 1.00 | −5.67 |
| 13 | BT | 6 | 1.047 ± 0.10 | 6.72 ± 0.24 | 24.30 ± 10.25 | |
| 14 | BT | 10 | 1.233 ± 0.03 | 7.10 ± 0.00 | 25.75 ± 0.78 | −6.28 |
| 15 | | 7 | 1.753 ± 0.06 | 7.15 ± 0.21 | 15.05 ± 0.49 | −14.00 |
| 16 | HA | 8 | 0.723 ± 0.15 | 7.25 ± 0.07 | 35.75 ± 2.62 | |
| 17 | HA | 7 | 0.864 ± 0.08 | 6.80 | 17.80 | −13.10 |
| 18 | HA | | 0.799 ± 0.03 | | | |
| 19 | HA | | 0.923 ± 0.15 | | | |
| 20 | HA | 9 | 2.876 ± 0.84 | 7.70 | 53.00 | −15.37 |
| 21 | HA | | 0.845 ± 0.08 | 12.20 | 5.80 | |
| 22 | HA | | 0.307 ± 0.11 | 6.40 | 25.50 | |
| 23 | HA | | 2.179 ± 0.09 | 6.30 | 23.70 | −25.25 |
| 24 | HA | | 3.251 ± 1.30 | 7.40 | 69.60 | −15.25 |
| 25 | HA | 9 | 1.664 ± 0.15 | 6.80 | 35.20 | −4.88 |
| 26 | HA | | 3.155 ± 0.07 | 7.10 | 90.00 | −10.25 |
| 27 | HA | | 3.066 ± 0.22 | 7.40 | 63.20 | −3.57 |
| 28 | HA | | 7.514 ± 1.29 | | | |
| 29 | HA | | 0.992 | 7.20 | 39.10 | |
| 30 | HA | | 1.446 | 7.50 | 40.20 | |
| 31 | HA | | 1.148 | 9.10 | 121.50 | |
| 32 | HA | | 0.872 ± 0.18 | 7.20 | 60.20 | |
| 33 | HA | | 0.546 | 7.40 | 13.70 | |
| 34 | HA | 7 | 0.472 | 6.90 | 8.00 | |
| 35 | HA | | 0.276 | 7.70 | 3.50 | |
| 36 | HA | | 0.356 | 7.10 | 2.50 | |
| 37 | HA | | 0.373 | 7.00 | 3.80 | |
| 38 | HA | | 0.343 | 6.60 | 10.80 | |

### Example 3

### Effect of Heating

Carbon dioxide enriched compositions containing water, nanostructures and CO₂ phase prepared according to various exemplary embodiments of the present invention were subjected to heating test so as to investigate the effect of plate heating on the IC content of the composition.

The enriched nanostructure composition of the present embodiments is interchangeable referred to in this example as “gas enriched nanostructure composition” abbreviated “GENC.”

### Material and Methods

Five GENC samples, enumerated 1-5 hereinbelow were prepared as described in Example 2 above. In sample Nos. 1 and 4, the core material was HA and the IC before the experiment was about 0.7 ppm; in sample Nos. 2 and 3, the core material was HA and the IC before the experiment was about 1.5 ppm, and in sample No. 5, the core material was BT and the IC before the experiment was about 1.9 ppm IC. Ultra pure water (UPW) was used as control.

Each sample was prepared in two replicates of 50 mL each.

All samples were heated for 2 hours on a hot plate preset to 60° C.

After 2 hours all samples were removed from the hot plate. The IC content of 1 replicate of each sample was measured using the autosampler mode of a TOC instrument. The second replicate of each sample was stored for 1 week closed on bench top in room temperature and no light protection. The IC of the stored samples was measured 1 week later using the autosampler mode of the TOC instrument.

Due to the IC results of the 1 week stored replicates a pH measurement was taken in order to rule out dissolution and ionization of CO₂.

### Results

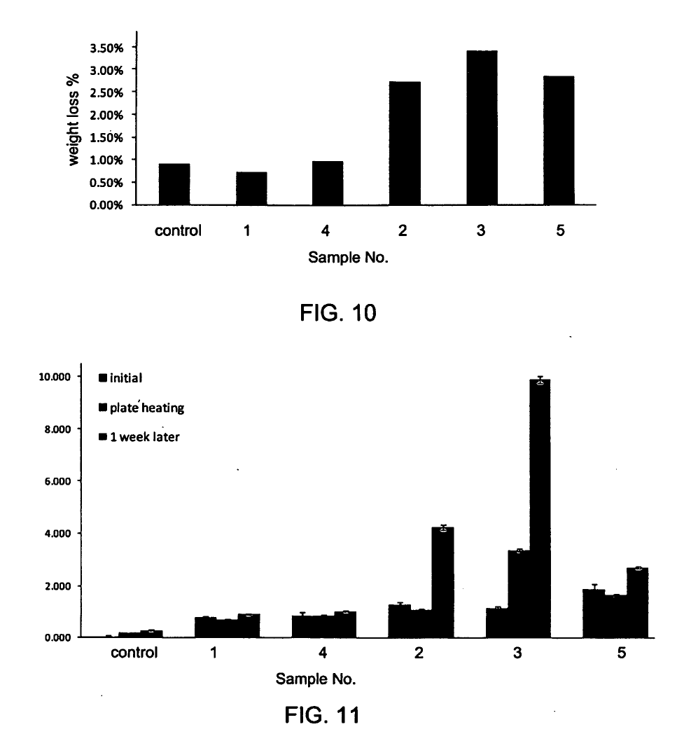

Table 3 and FIG. 10 present the weight losses after two hours of plate heating.

TABLE 3

| Sample | Initial Weight [g] | Weight Following Heating [g] | Weight Loss |
|---|---|---|---|
| Control | 48.911 ± 0.291 | 48.473 ± 0.119 | 0.9% |
| 1 | 48.946 ± 0.191 | 48.595 ± 0.174 | 0.72% |
| 2 | 49.572 ± 0.290 | 49.091 ± 0.422 | 0.97% |
| 3 | 49.008 ± 0.106 | 47.668 ± 0.320 | 2.74% |
| 4 | 48.822 ± 0.790 | 47.146 ± 0.434 | 3.43% |
| 5 | 49.096 ± 0.183 | 47.698 ± 0.770 | 2.85% |

Table 4 and FIG. 11 present the IC content immediately following plate heating and one week after plate heating.

TABLE 4

| | IC Content [ppm] | | |
|---|---|---|---|
| Sample | Before Heating | Following Heating | Week After Heating |
| Control | 0.029 ± 0.038 | 0.181 ± 0.009 | 0.287 ± 0.008 |
| 1 | 0.799 ± 0.022 | 0.710 ± 0.012 | 0.930 ± 0.017 |
| 2 | 0.872 ± 0.126 | 0.878 ± 0.013 | 1.057 ± 0.016 |
| 3 | 1.307 ± 0.091 | 1.101 ± 0.012 | 4.241 ± 0.092 |
| 4 | 1.179 ± 0.063 | 3.368 ± 0.062 | 9.885 ± 0.128 |
| 5 | 1.900 ± 0.178 | 1.652 ± 0.028 | 2.706 ± 0.037 |

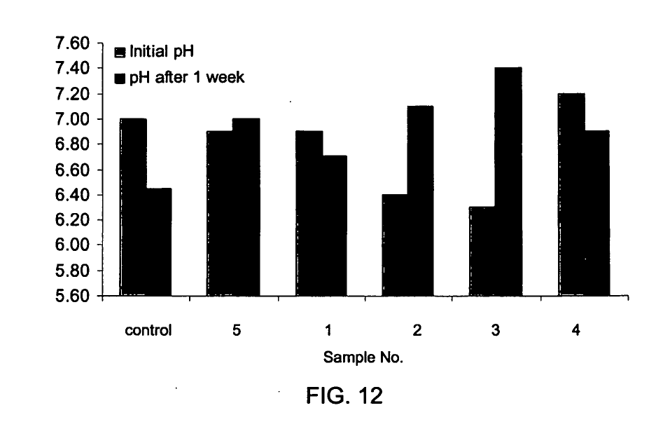

Table 5 and FIG. 12 present the pH values one week after plate heating.

TABLE 5

| | | pH after |
|---|---|---|
| Sample | Initial pH | 1 week |
| Control | 7.0 | 6.45 |
| 5 | 6.9 | 7.0 |
| 1 | 6.9 | 6.7 |
| 2 | 6.4 | 7.1 |
| 3 | 6.3 | 7.4 |
| 4 | 7.2 | 6.9 |

Tables 3-5 and FIGS. 10-12 demonstrate that:

Sample Nos. 1 and 4 that had relatively low initial IC content had identical weight loss and similar IC content change which are also similar to those of the control sample.

Sample Nos. 2, 3 and 5 which had a higher initial IC content had a similar weight loss which was different than the weight loss of the other samples and the control.

In terms of IC content changes measured after 2 hours of 60° C. heating on a plate, all samples except sample No. 2 had the same behavior of a mild change in the IC content values.

The IC content changes of sample Nos. 1 and 4 and of the control measured after 2 hours of 60° C. heating on a plate and one week of storage in room temperature were similar (increase of 0.1-0.2 ppm).

The IC content changes of sample Nos. 2, 3 and 5 measured after 1 week of storage in room temperature were also similar. In each of these samples there was a large increase in the IC content compared to the initial values and to the values measured after 2 hours of heating.

This experiment demonstrates that the compositions with higher IC content lose more weight and therefore evaporate more water during heating compared to the compositions with low IC content. Subsequently to the heat treatment the IC content of the compositions with high initial IC content raised after storage. This rise was not due to dissolution of atmospheric CO₂ since the pH values of these samples was also raised. This indicates the composition of the present embodiments has a stable or meta stable gas phase. The raise in the IC content is more pronounced in compositions with initial IC content which is above 1 ppm. Thus, the technique of hot plate is suitable method for concentrating and reclamation the enriched composition of the present embodiments.

### Example 4

### Prototype CO₂ Recycling Apparatus

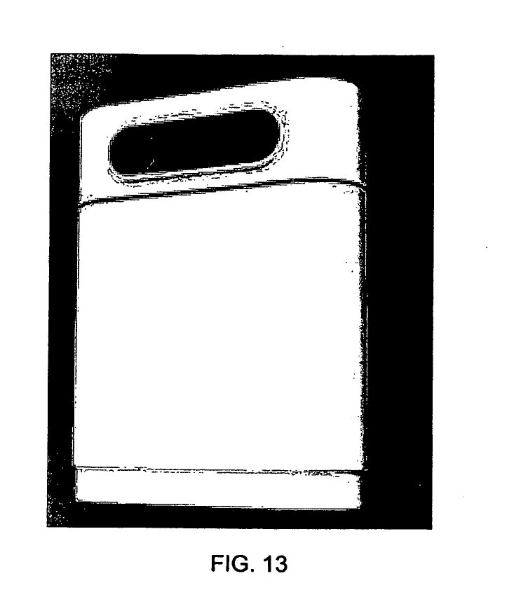

Three prototype apparatus which can be used for recycling CO₂ have been manufactured in accordance with embodiments of the present invention. The apparatus included a CO₂ enriched nanostructure composition prepared according to some embodiments of the present invention. The apparatus further included an excitation device in the form of a radiofrequency generator and an antenna. The antenna was disposed in a sleeve having an outlet as described above. An outlet valve and an inlet valve were used for controlling release and collection of CO₂ as described above. The three prototype apparatus differ in their capacity. The volumes of the liquid chambers (see 42 in FIG. 5) for the first, second and third apparatus were 50 ml, 100 ml and 200 ml, respectively. An image of the second prototype apparatus is shown in FIG. 13.

The prototype apparatus were subjected to CO₂ concentration level tests. For each apparatus, the concentration level of CO₂ was measured and recorded at intervals of 30 seconds at the outlet of the apparatus. The tests were conducted while the outlet valves were operated intermittently according to several scenarios. The operation scenarios are denoted below in close/open ratios. The notation X/Y refers to an operation scenario in which the valve is closed for X seconds and opened for Y seconds. In all the experiments, the operation scenario of the excitation device was according to an active/inactive ratio of 1/10.

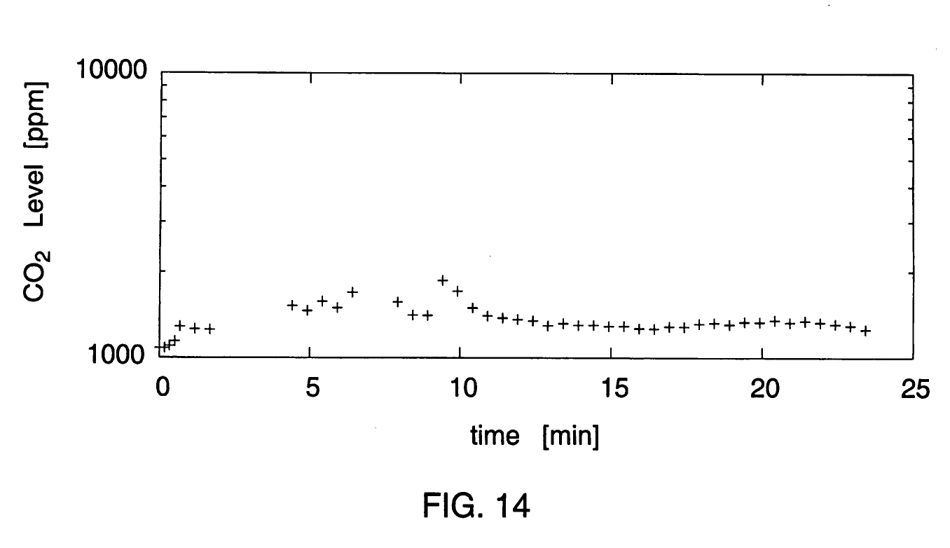

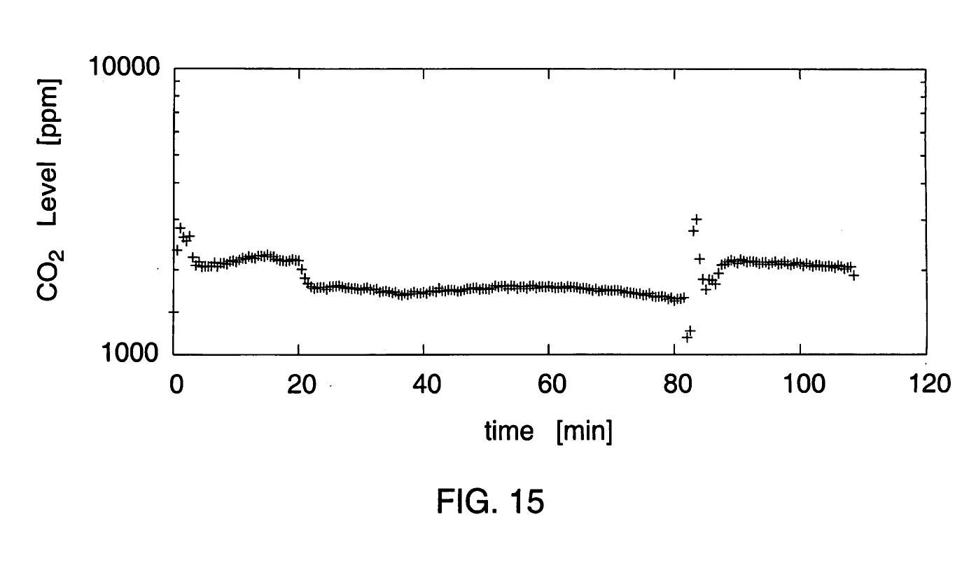

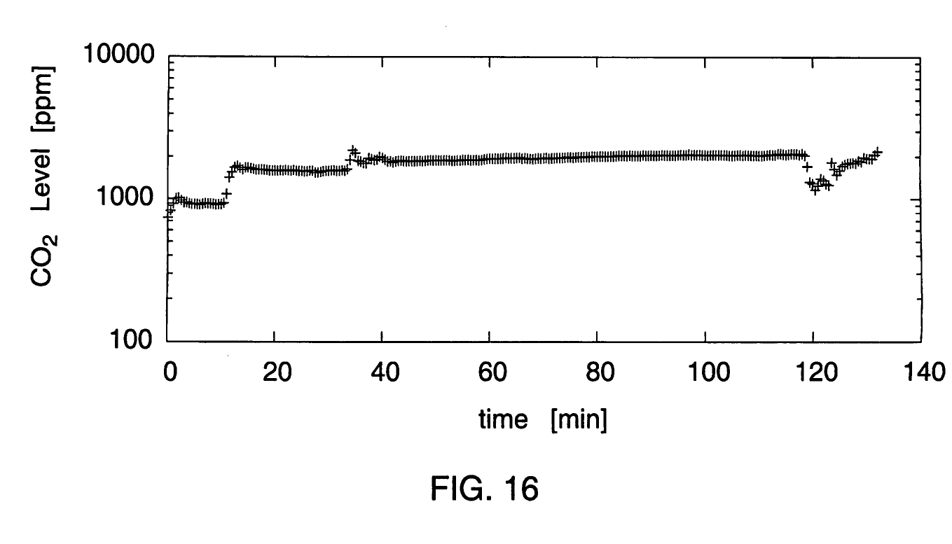

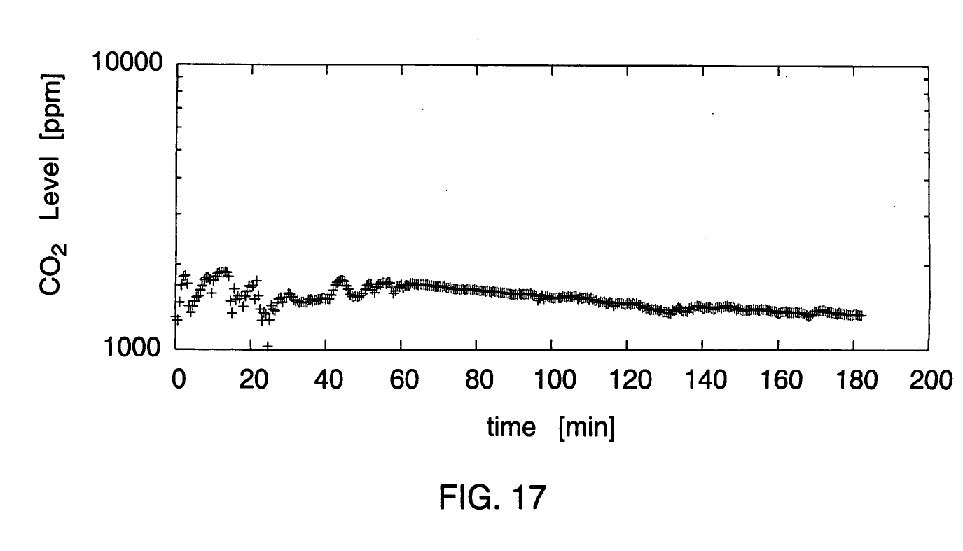

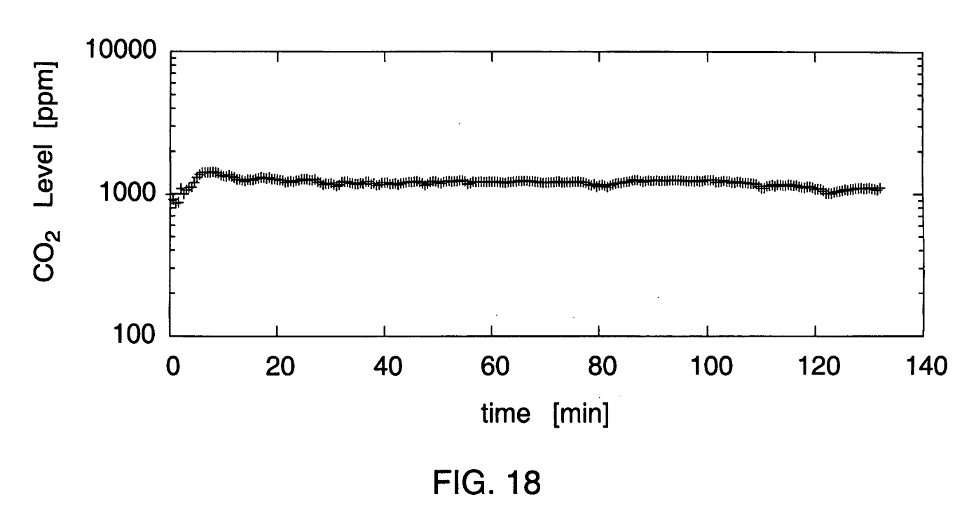

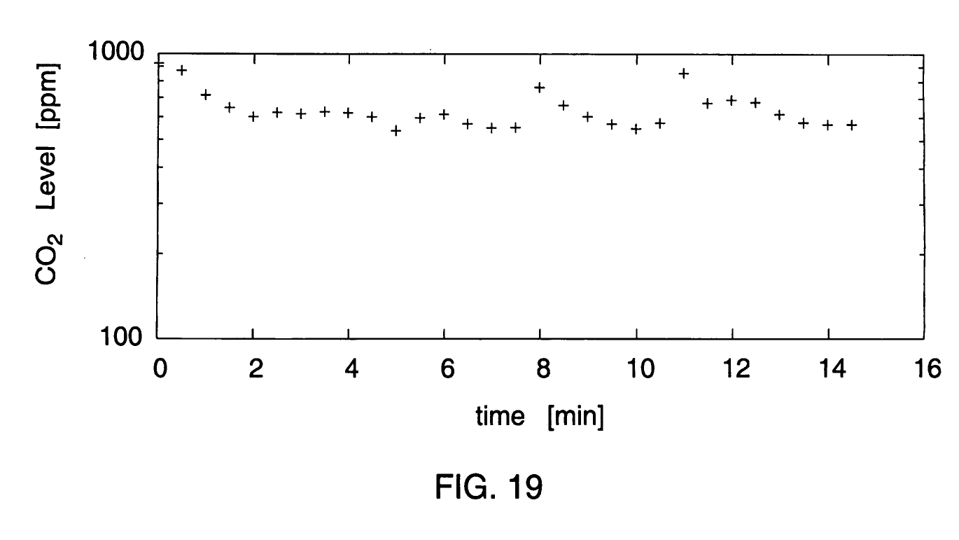

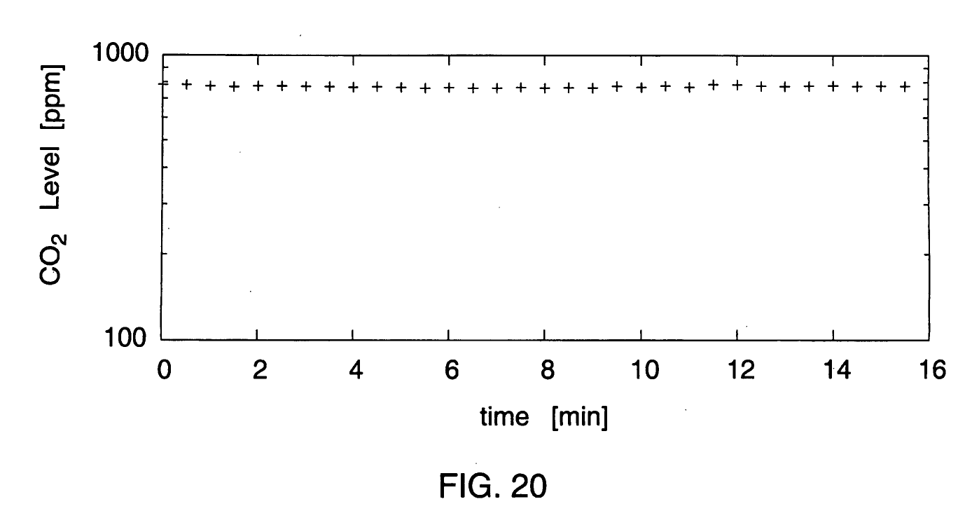

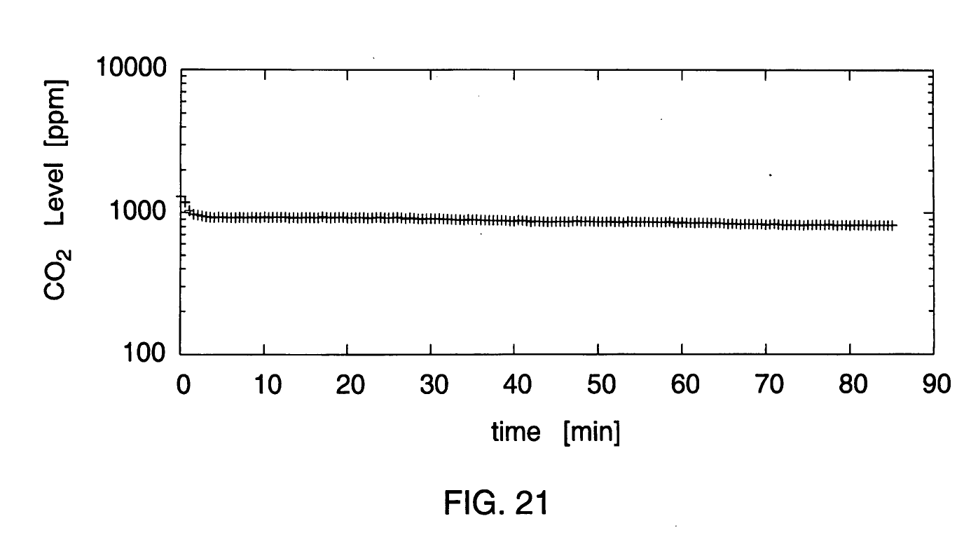

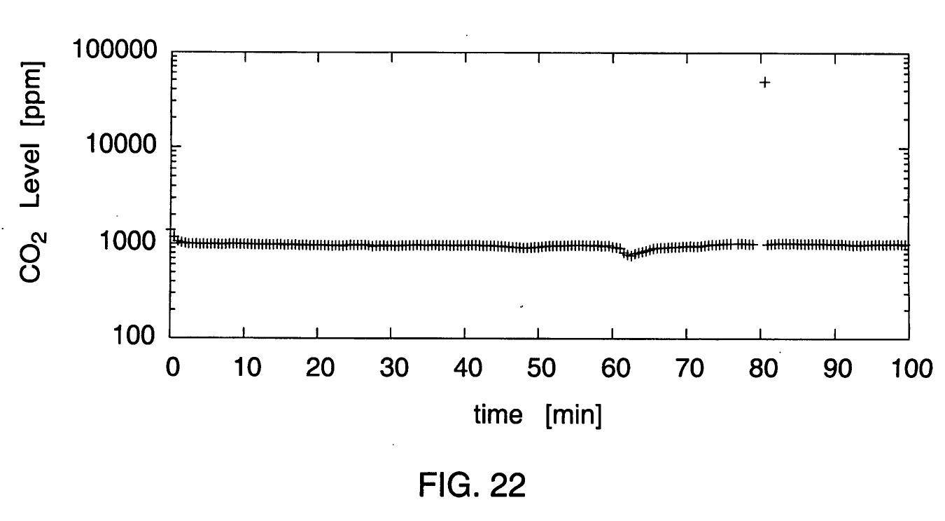

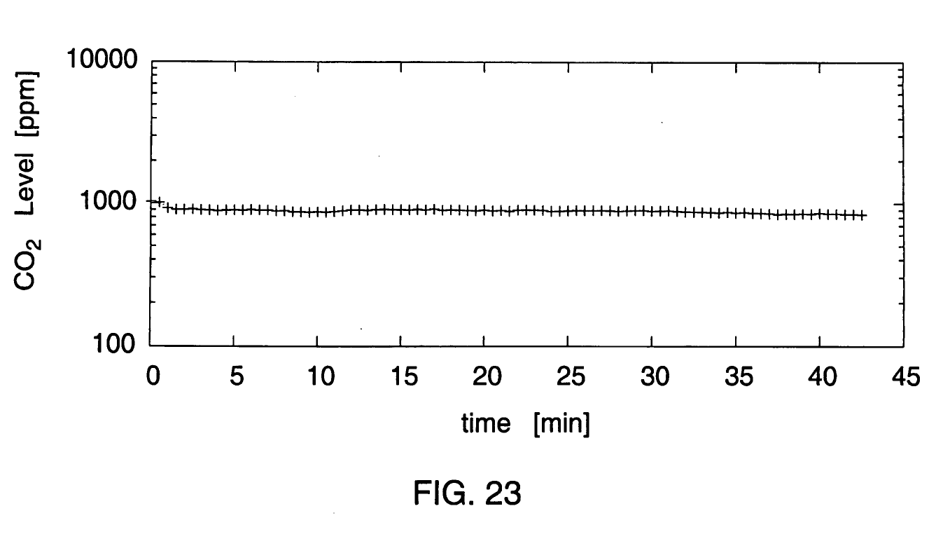

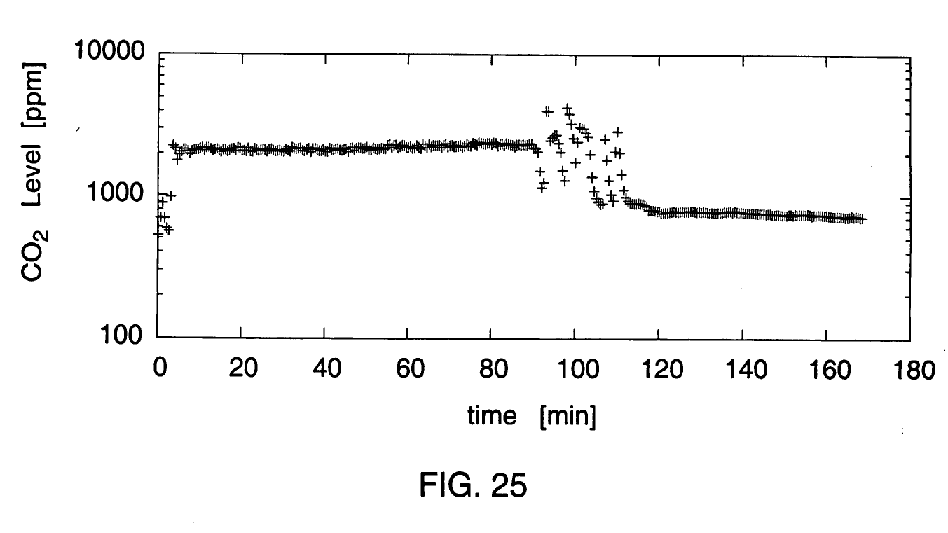

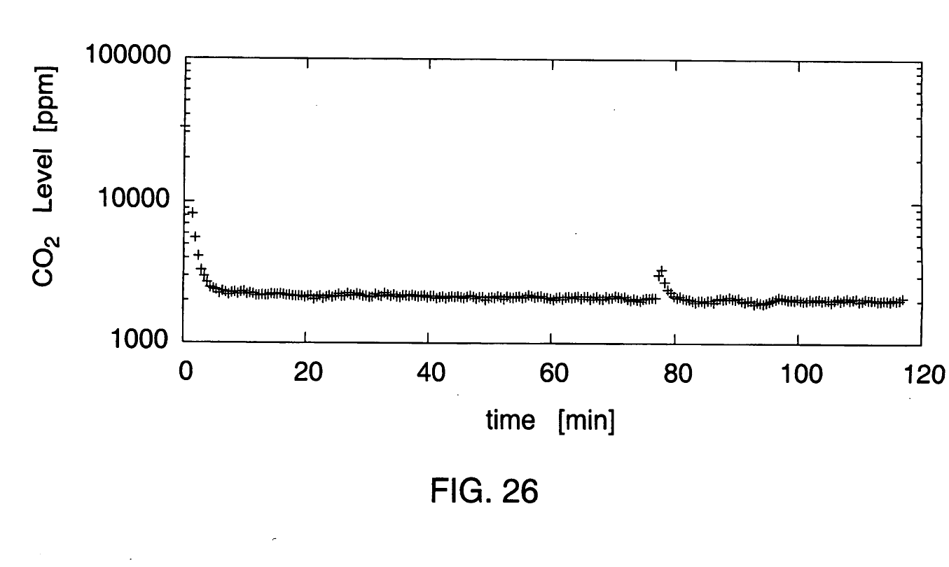

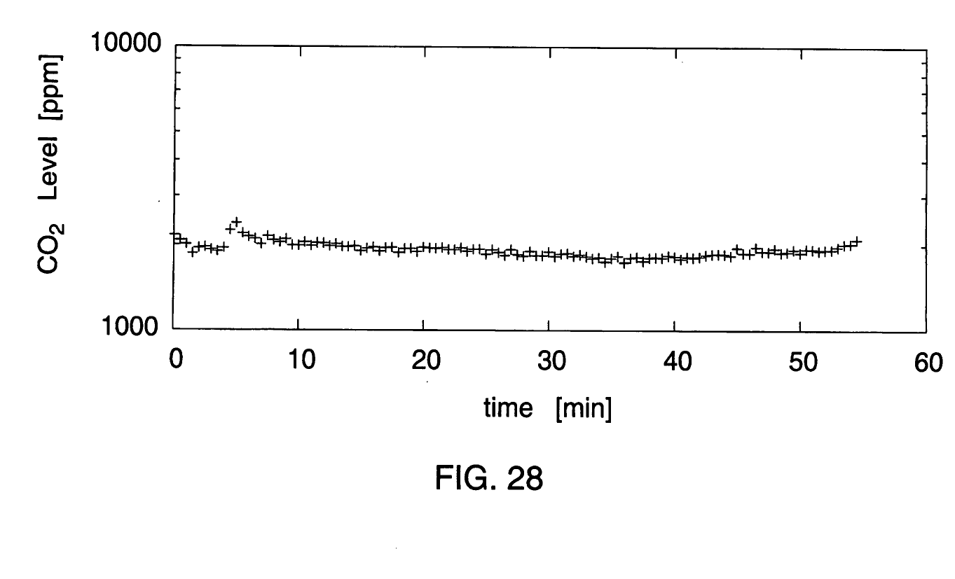

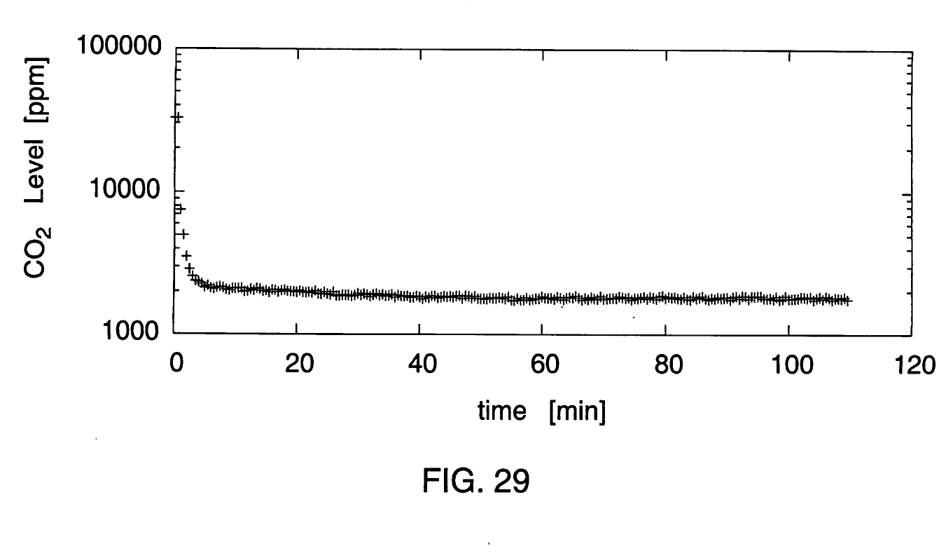

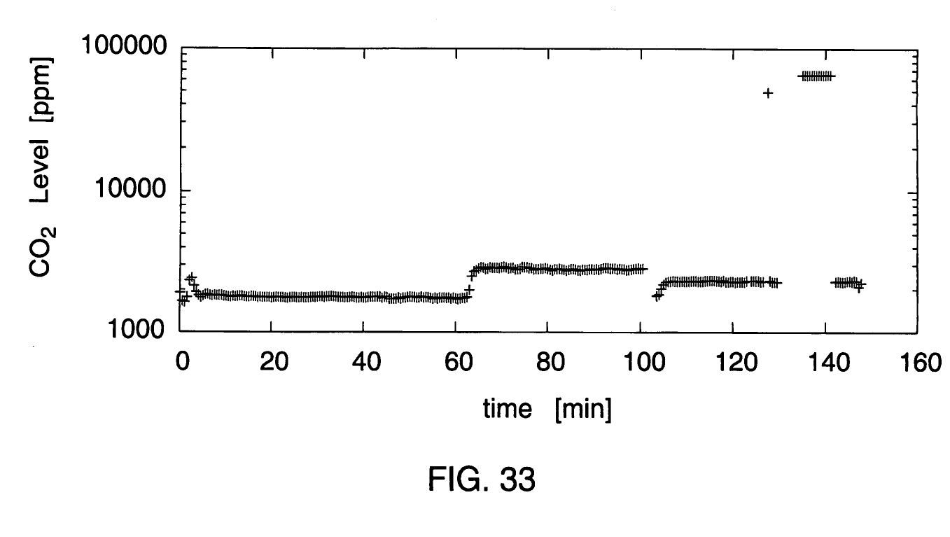

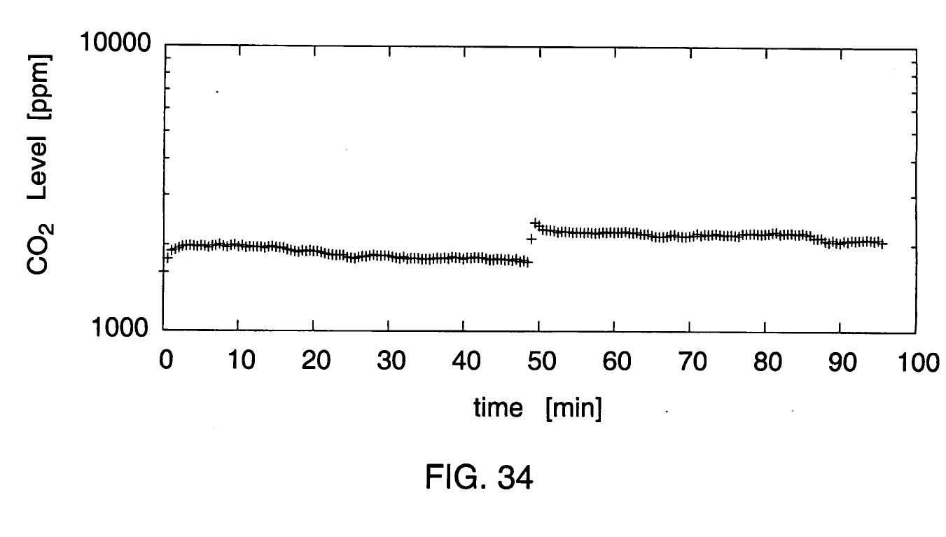

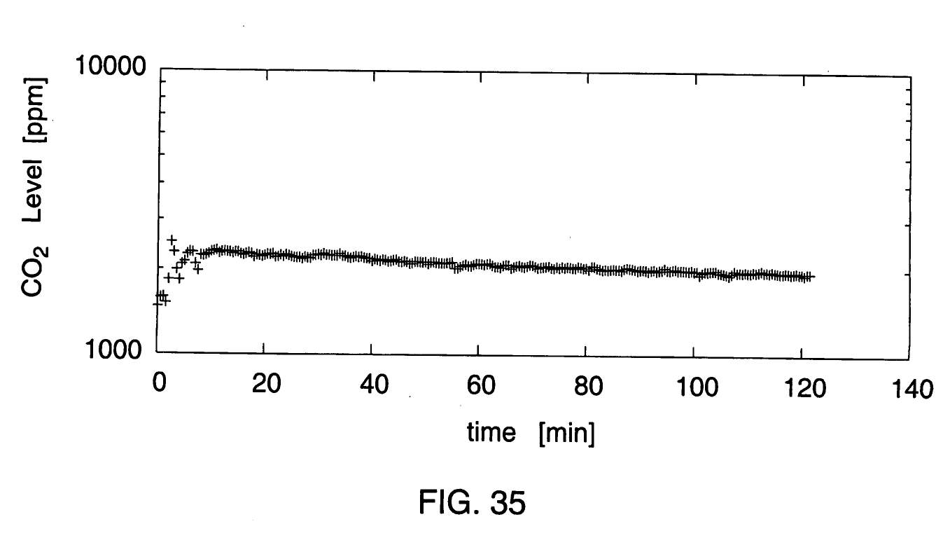

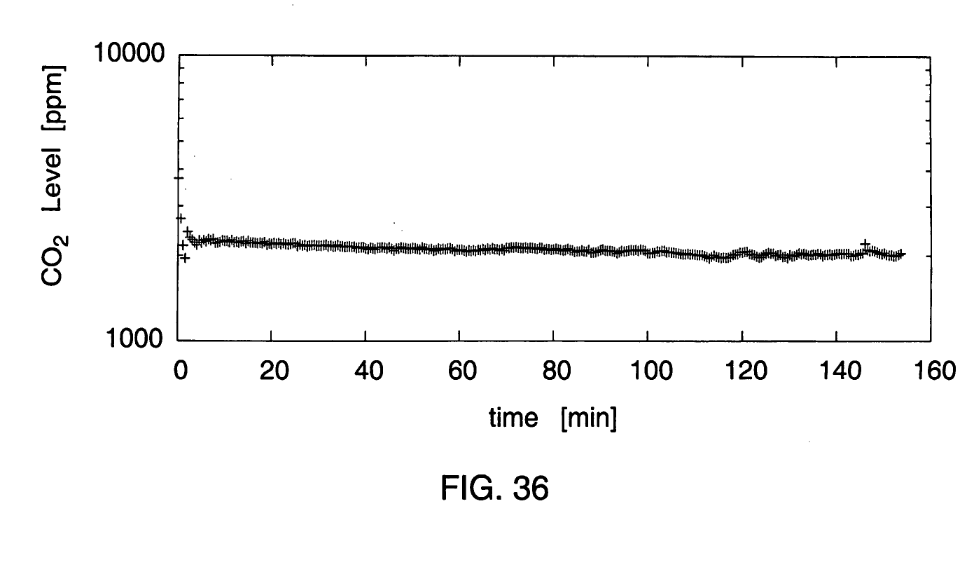

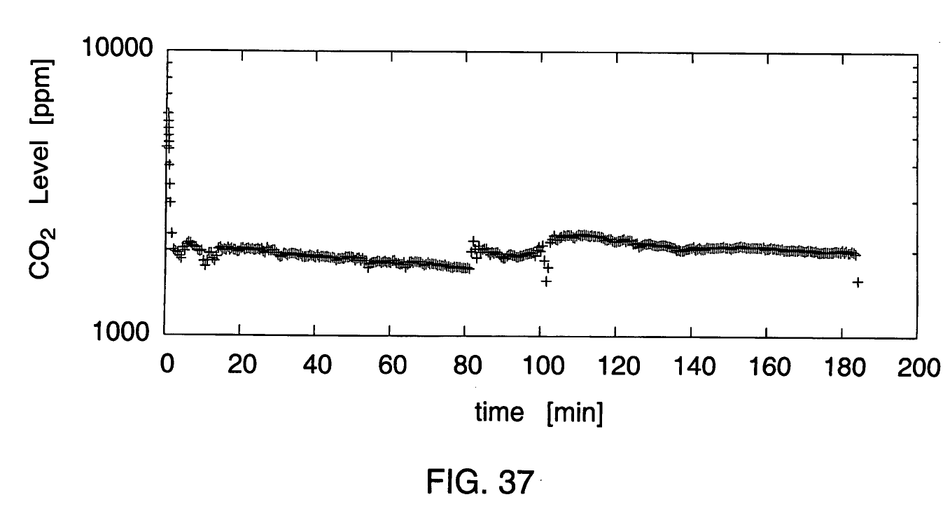

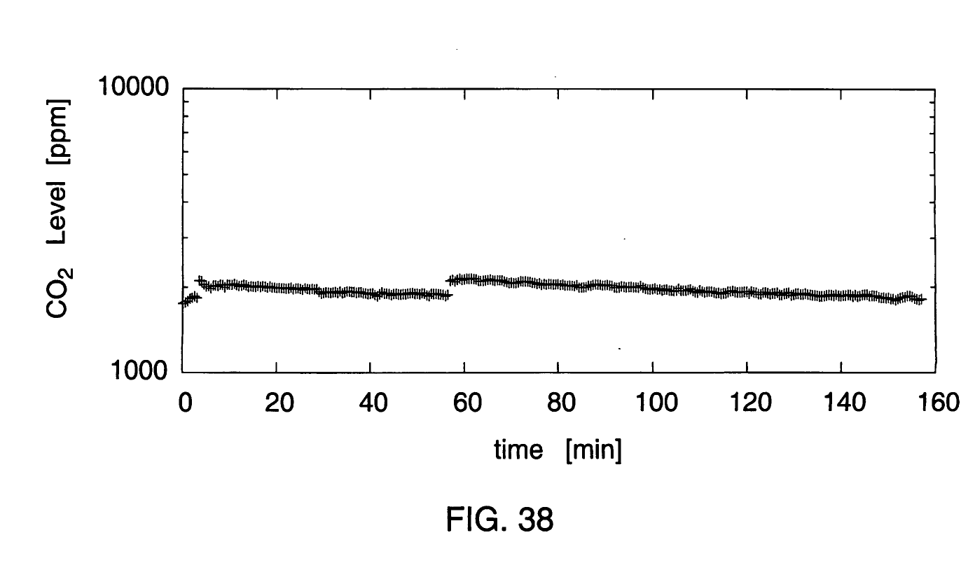

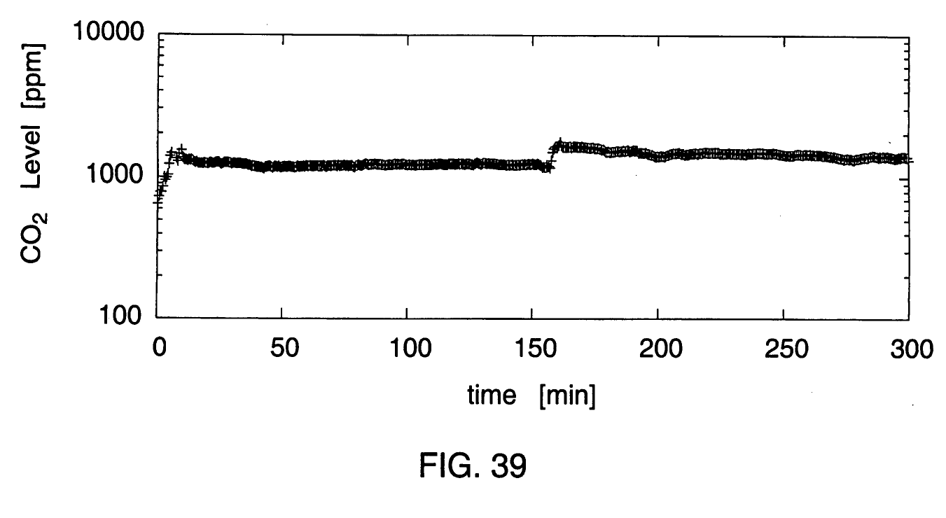

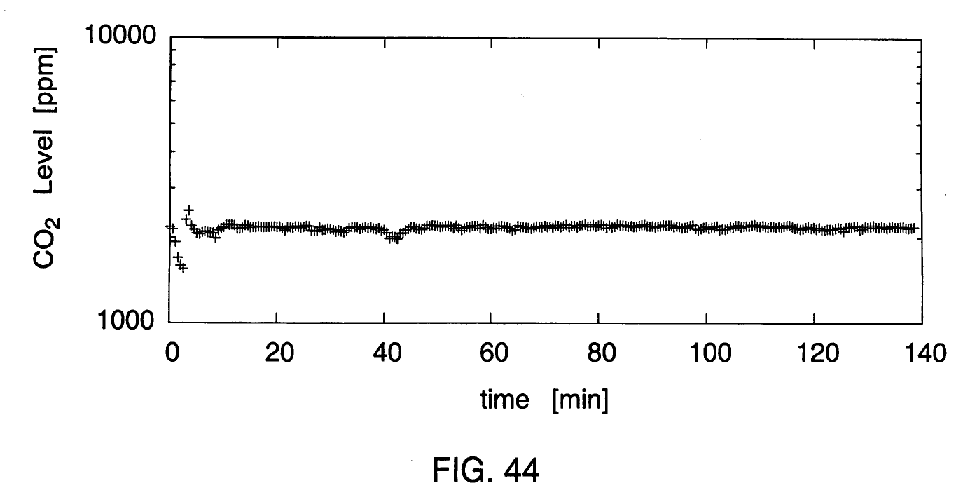

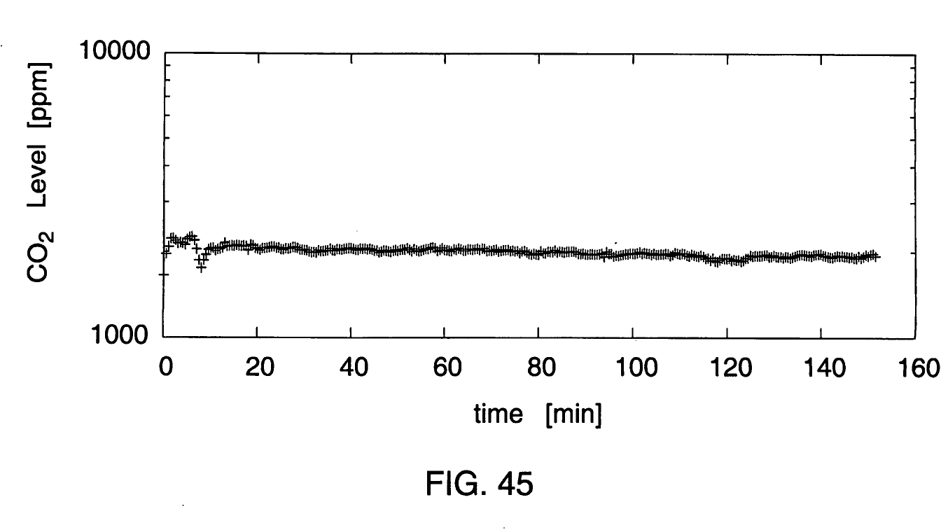

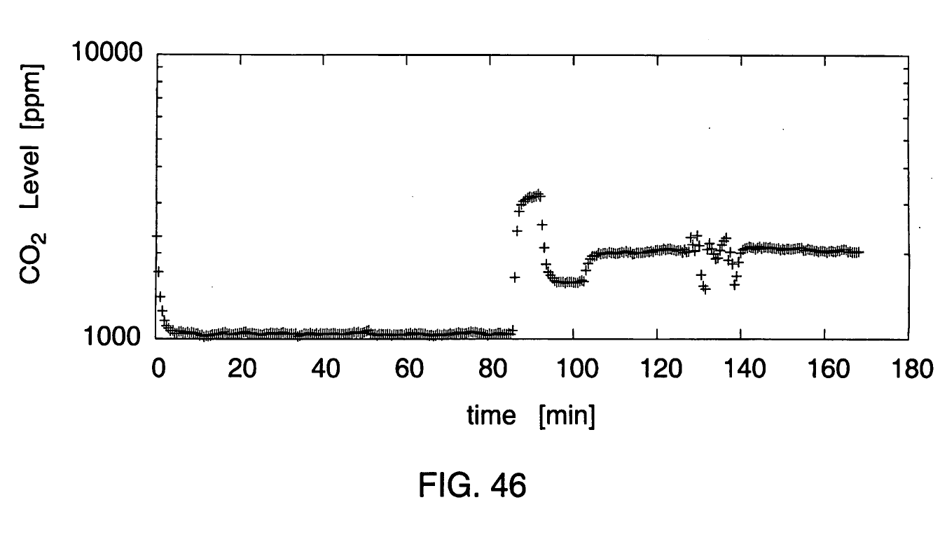

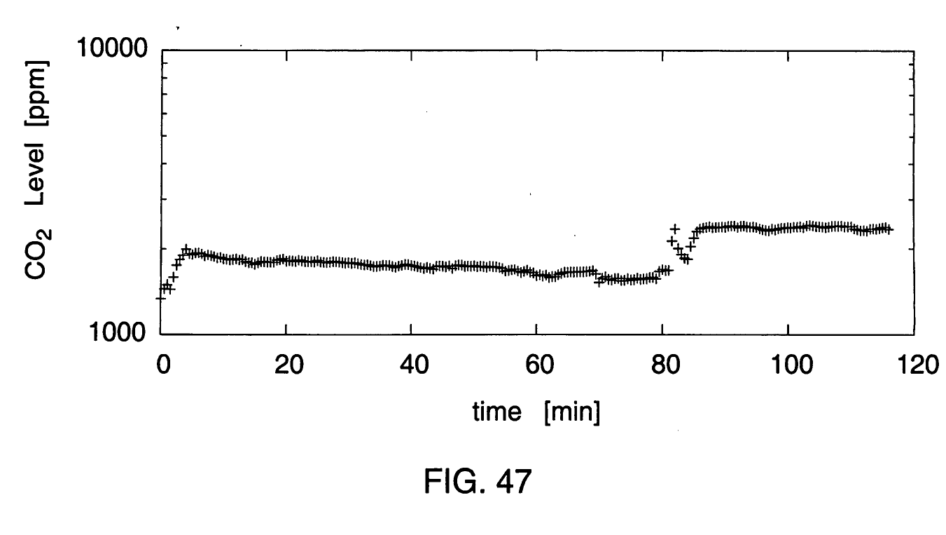

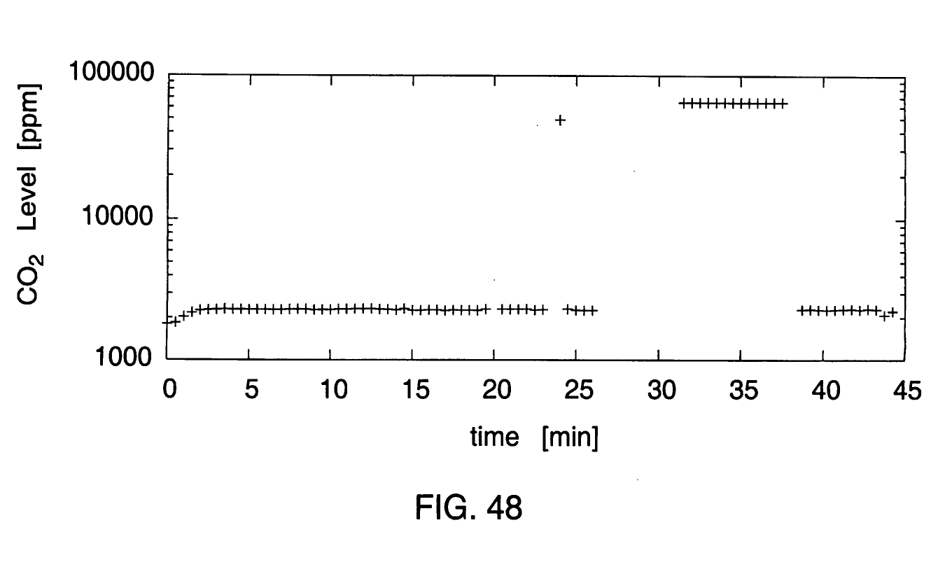

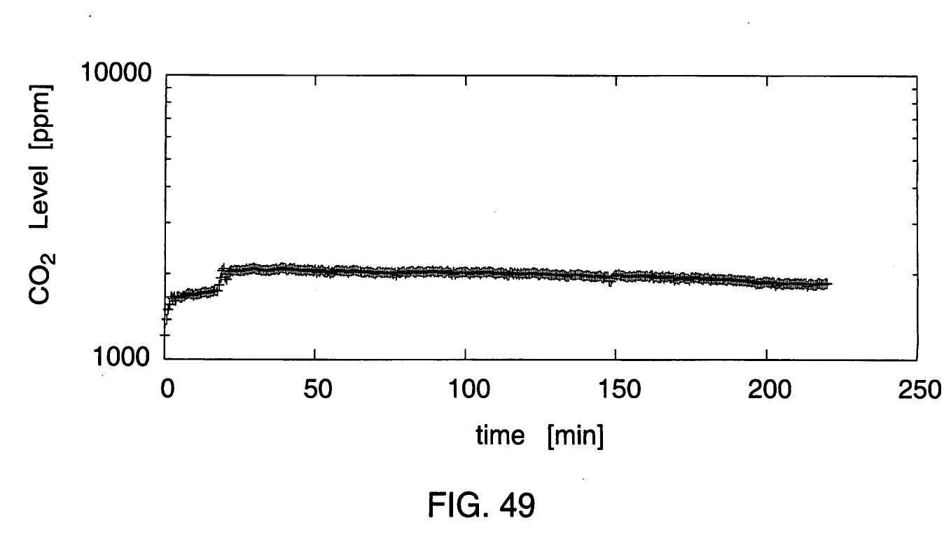

The results are presented in FIGS. 14-53 as plots of CO₂ concentration levels as a function of time, where the CO₂ levels are presented in ppm by volume and the time is presented in minutes. All the experiments begin at t=0.

FIGS. 14-23 show experimental results for the first apparatus. The results shown in FIGS. 14-23 correspond to operation scenarios of 30/1, 20/1, 15\1, 10\1, 5\2, 3\2, 2\2, 1\3, 1\3 and 1\4, respectively.

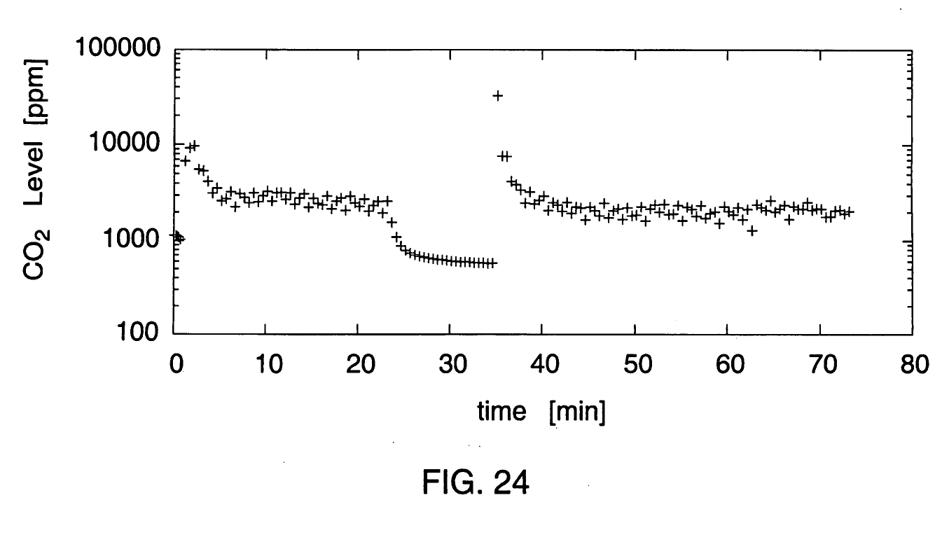

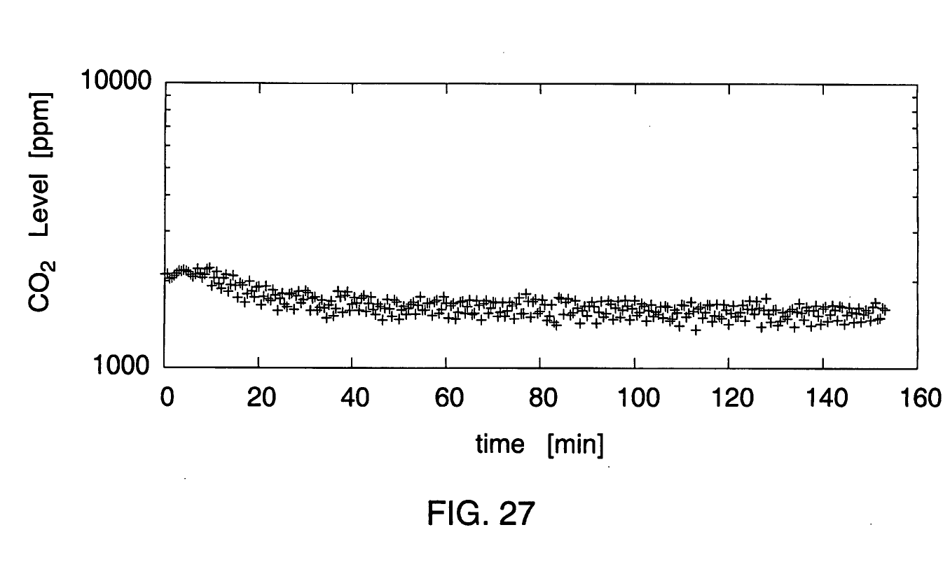

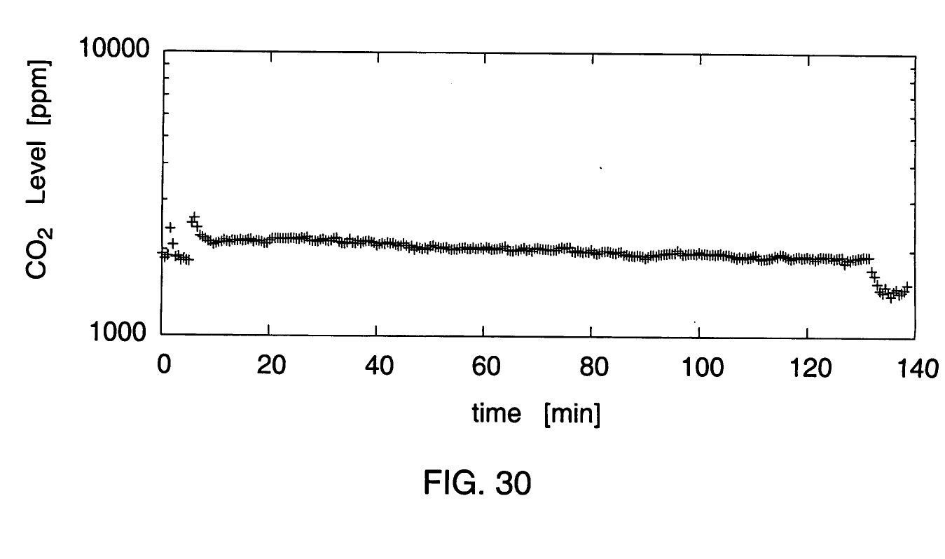

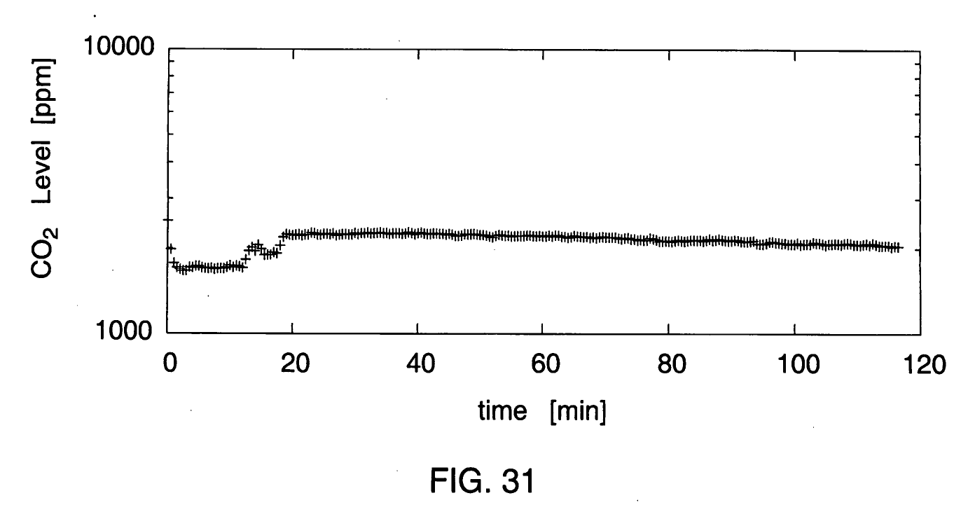

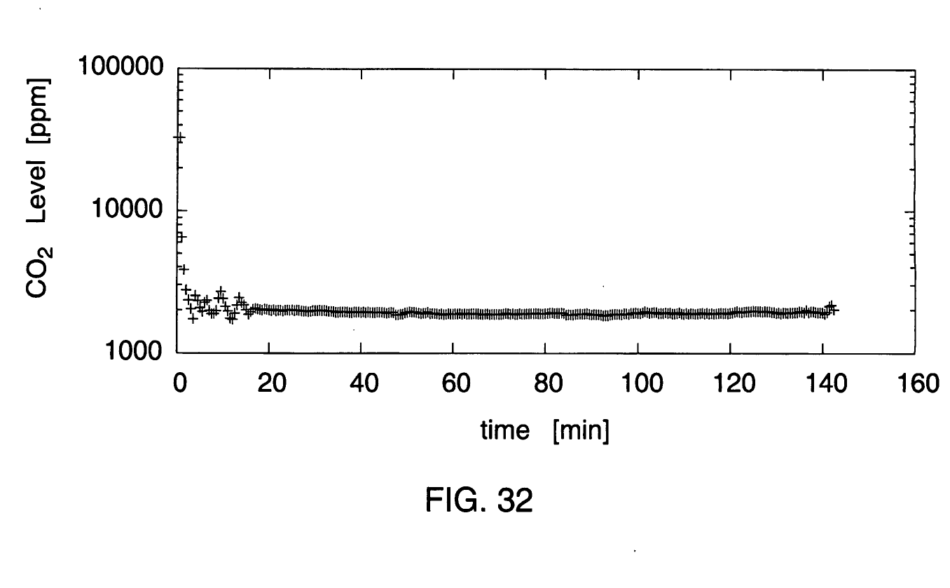

FIGS. 24-39 show experimental results for the second apparatus. The results shown in FIGS. 24-26 correspond to operation scenarios of 30\1; the results shown in FIGS. 27-29 correspond to operation scenarios of 20\1; the results shown in FIGS. 30-31 correspond to operation scenarios of 15\1; and the results shown in FIGS. 32-39 correspond to operation scenarios of 12\1, 10\1, 8\1, 5\2, 3\2, 2\2, 1\2 and 1\3 respectively.

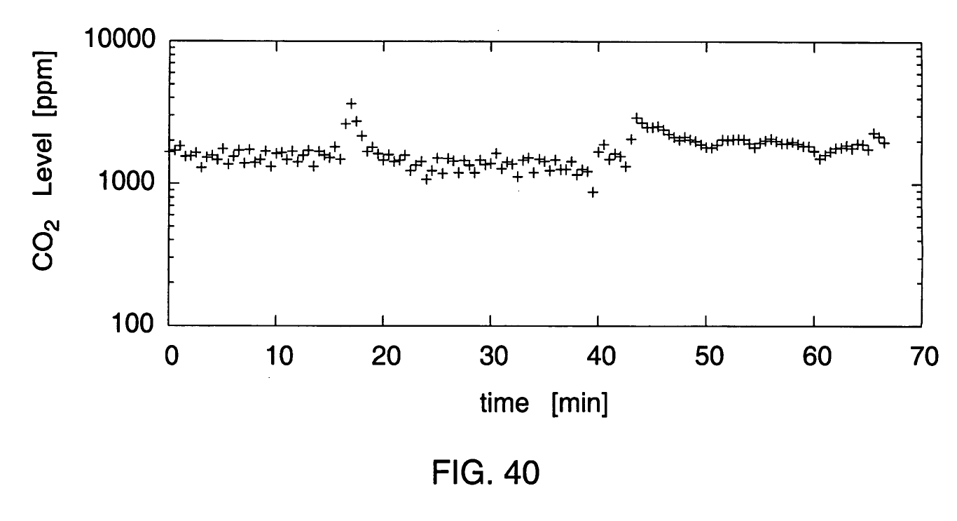

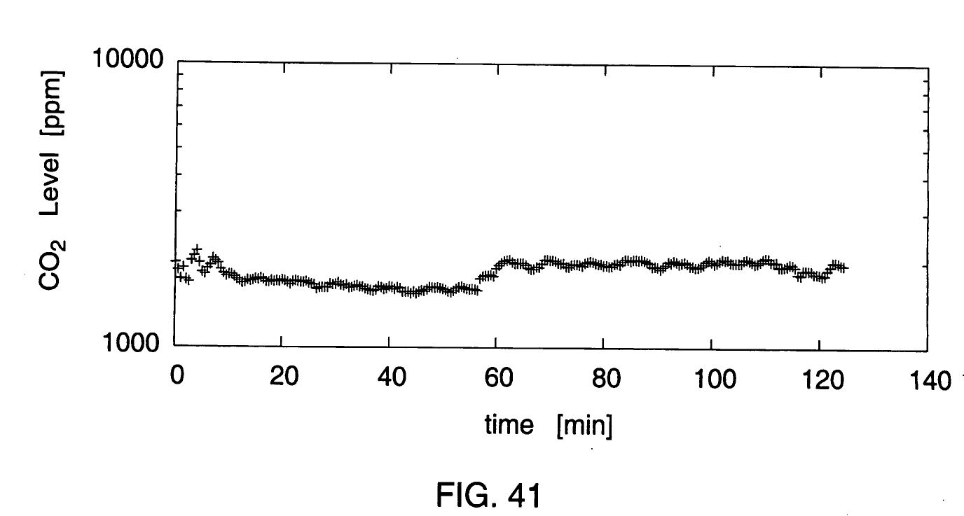

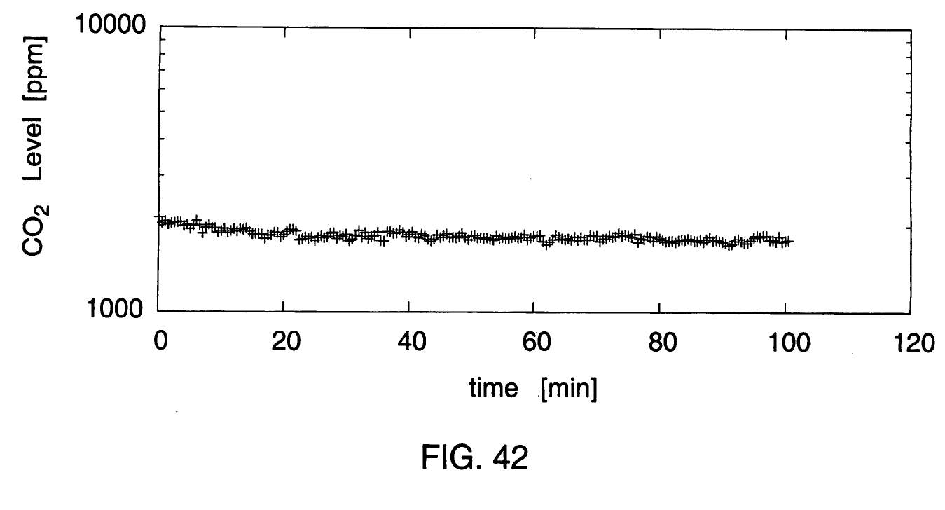

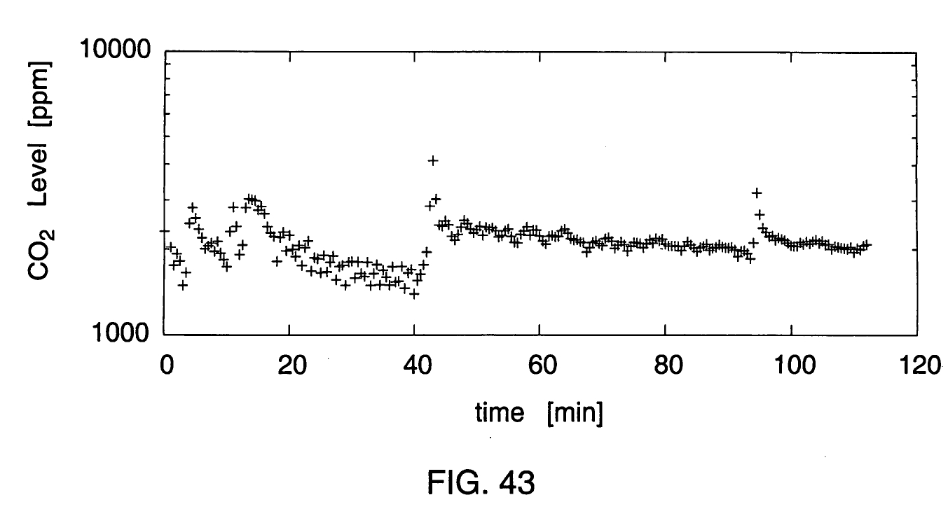

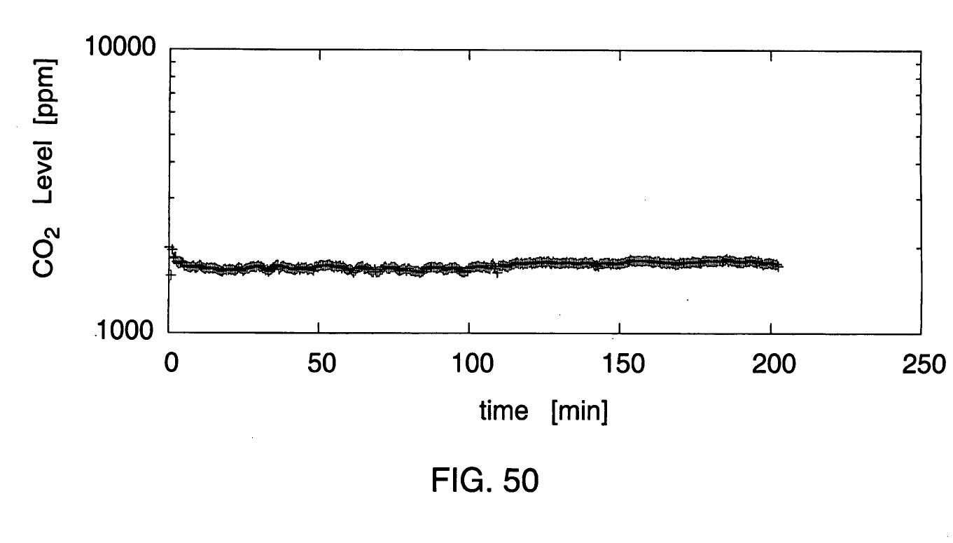

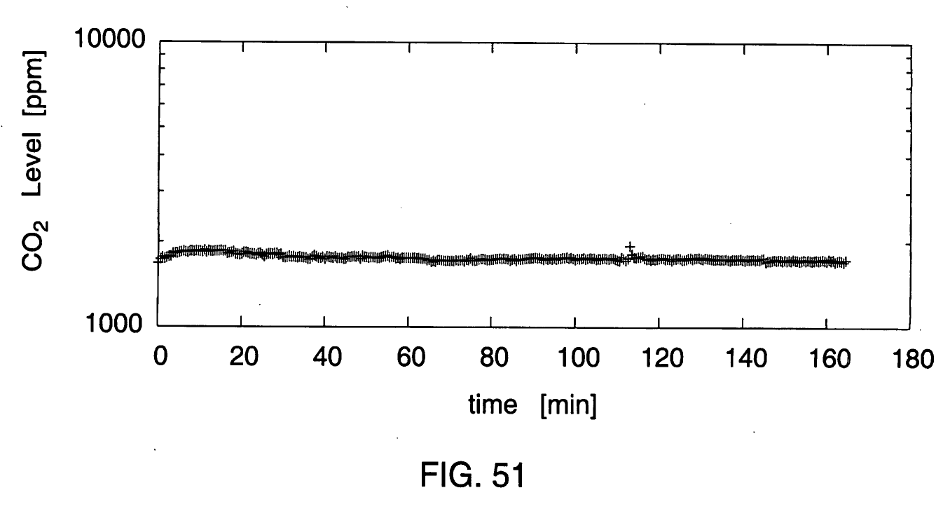

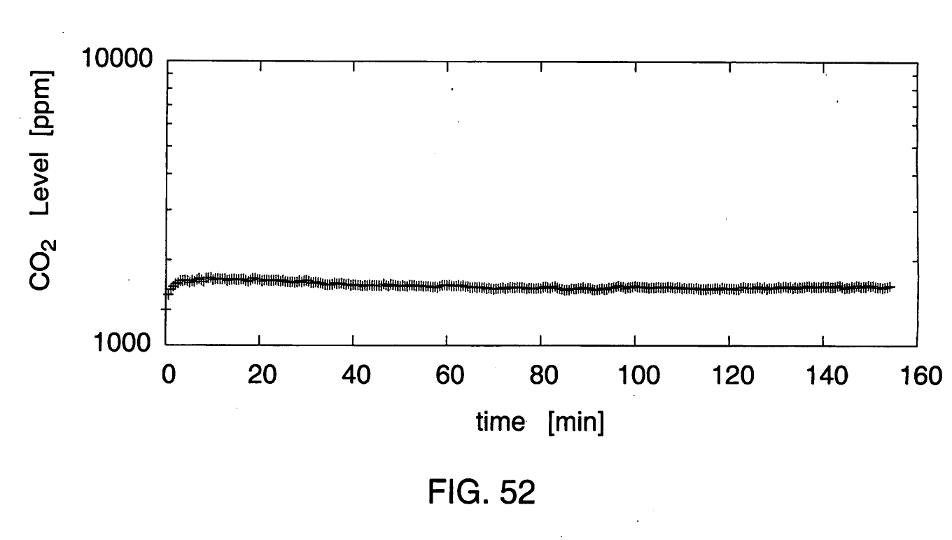

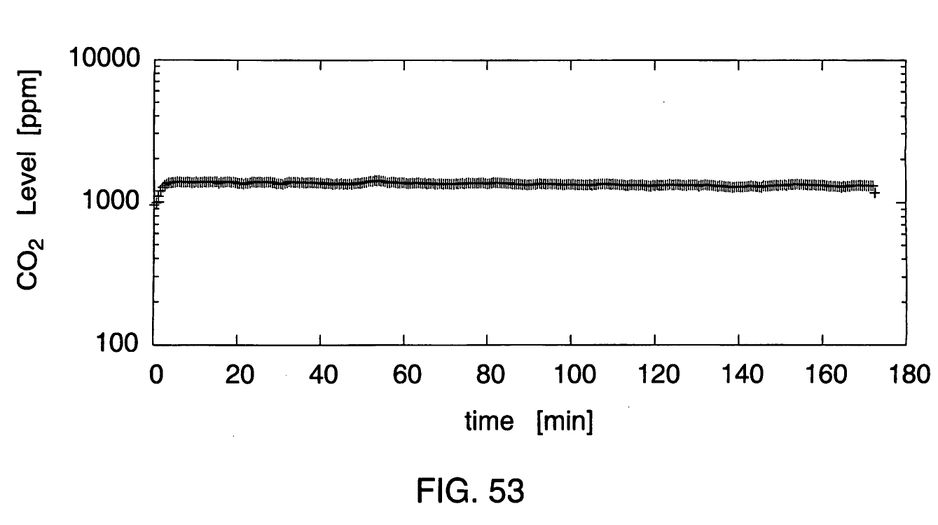

FIGS. 40-53 show experimental results for the third apparatus. The results shown in FIG. 40 correspond to an operation scenario of 30\1; the results shown in FIGS. 41-42 correspond to operation scenarios of 20\1; the results shown in FIGS. 43-49 correspond to operation scenarios of 15\1; the results shown in FIG. 50 correspond to an operation scenario of 10\1; the results shown in FIG. 51 correspond to an operation scenario of 5/2; and the results shown in FIGS. 52-53 correspond to an operation scenario of 1/3.

The results presented above demonstrate that a sufficiently high concentration of CO₂ can be achieved for many operation scenarios. As shown the CO₂ concentration levels at the outlet of the prototype apparatus are considerably above ambient levels which are approximately less than 300 ppm by volume. Generally, the prototype apparatus generate a local concentration which is of the order of 1000 ppm by volume. In several experiments, instantaneous bursts of extremely high levels (above 10,000 ppm by volume) were observed.

### Example 5

### Solid-Fluid Coupling

This Example describes a prophetic experiment for investigating the coupling of the surrounding fluid molecules to the core material Cryogenic-temperature transmission electron microscopy (cryo-TEM), which is a modern technique of structural fluid systems. The analysis can involve the following steps in which in a first step, the enriched nanostructure composition of the present embodiments can be cooled ultra-rapidly, so that vitreous sample was provided, and in a second step the vitreous sample can be examined in via TEM at cryogenic temperatures. Striations surrounding the nanostructures can suggest a crystalline structure thereof, and dark corona can indicate an ordered structure of the fluid molecules surrounding the core, so that the entire nanostructure is in a steady physical state.

### Example 6

### Optical Activity

This Example describes a prophetic experiment for investigating signatures of induced long range order. To this end, the optical activity (in terms of circularly and elliptically polarized light) of the enriched nanostructure composition of the present embodiments can be measured using the Circular Dichroism (CD) method.

CD spectroscopy aims to detect absorption differences between left-handed and righthanded (L and R) polarized lights passed through aqueous solutions. Such differences can be generated from optically active (chiral) molecules immersed in water, distribution of molecules or nanoparticles or any other induced ordered structures in the water or solutions. The measurements can be performed using a Jasco K851 CD polarimeter at room temperature. DDW can be used as the baseline.

The spectrum can be scanned between 190 nm and 280 nm using 1 nm and 10 seconds increments. In order to increase sensitivity and resolution a very long optical pathway can be ensured using a 10 cm quartz cuvette (compared to 1 mm or smaller in regular mode of operation).

Existence of non vanishing signal in the CD spectra of the enriched nanostructure composition of the present embodiments can indicate formation of long range orientational order therein. Such long range order can be formed by the network of nanoparticles and nanobubbles.

### Example 7

### Effect of Dye

This Example describes a prophetic experiment for investigating the interaction of the enriched nanostructure composition of the present embodiments with dye. A enriched nanostructure composition, manufactured as further detailed above can be dyed with a Ru based dye (N3) dissolved in ethanol.

One cuvette containing the enriched nanostructure composition of the present embodiments can be exposed to the dye solution for 24 hours. A second cuvette containing the enriched nanostructure composition can be exposed to the following protocol: (i) stirring, (ii) drying with air stream, and (iii) dying. Two additional cuvettes, containing pure water can be subjected to the above tests as control groups.

Changes of the dye color in the enriched nanostructure composition of the present embodiments in contrast to the case of pure water can indicate interaction with the nanostructures which affects the dye spectrum by either changing the electronic structure or by dye oxidation.

### Example 8

### High g Centrifugation

This Example describes a prophetic experiment for investigating the effect of the enriched nanostructure composition of the present embodiments on of high g centrifugation on the enriched nanostructure composition of the present embodiments.

Tubes containing the enriched nanostructure composition of the present embodiments can be centrifuged at high g values (about 30 g). Integrated light scattering (ILS) measurements of the enriched nanostructure composition of the present embodiments after centrifugation can then be taken. Different records at the lower portion and upper of the tubes, can indicate that the nanostructures have a specific gravity which is lower than the specific gravity of the host liquid (water).

### Example 9

### Bacteriophage Reaction

This Example describes a prophetic experiment for investigating the effect of the enriched nanostructure composition of the present embodiments on bacteriophage typing. Bacteriophages of a standard international kit for phage typing of staphylococcus aureus (SA), can be examined (e.g., bacteriophages No. 6 and 83A). Media for agar plates can be Nutrient agar Oxoid No2 (catalog number CM 67 Oxoid Ltd.)+CaCl₂. After autoclave sterilization, 20 ml of CaCl₂ can be added for each liter of medium. Media for liquid cultures can be Nutrient Broth No2 Oxoid: 28 gr/1 liter.

Each bacteriophage can be tested at 1 and 100 RTD (Routine Test Dilution). Each phage can be propagated in parallel in control and in tested media based on the enriched nanostructure composition of the present embodiments. The bacteriolysis surface can be measured using computerizes “Sketch” software for surface area measurements. Analysis-of-variance (ANOVA) with repeated measures can be used for optic density analysis, and 2 ways ANOVA for lysis surface area measurements using SPSS™ software for Microsoft Windows™.

An increase in phage reaction area with the enriched nanostructure composition of the present embodiments compared to control can demonstrate that the enriched nanostructure composition of the present embodiments has identical trends of effect the phages.

In the RTD test, a different trends in time between the control and the enriched nanostructure composition of the present embodiments can demonstrate the effect of the enriched nanostructure composition of the present embodiments on phage reaction.

### Example 10

### Phage-Bacteria Interaction

This Example describes a prophetic experiment for investigating the effect of the enriched nanostructure composition of the present embodiments on Lambda (λ) phage. λ phage is used in molecular biology for representing the genome DNA of organisms. The experiments can rely on standard λ phage interaction applications. The materials in the test groups can be prepared with the enriched nanostructure composition of the present embodiments as a solvent. The materials in control groups can be prepared as described hereinbelow. The pH of the control groups can be adjusted to the pH of the enriched nanostructure composition of the present embodiments, which was between 7.2 and 7.4.

1) LB Medium
10 g. of Bacto Tryptone, 5 g of Yeast extract, 10 g of NaCl can be dissolved in 1000 ml of distilled water, and then sterilized by autoclave (121° C., 1.5 atm for 45 minutes).

2) LB Plates
15 g of Bacto Agar can be added to 1000 ml of LB medium, mixed and autoclaved as described above. After cooling to 50° C., the medium can be poured into sterile plastic plates. The plates can be pre-incubated for two days before use.

3) Top Agarose 0.7%
100 ml of LB medium can be mixed with 0.7 g of chemically pure, electrophoresis grade agarose (from Difco or other supplier), and then sterilized by autoclave (121° C., 1.5 atm during 45 minutes).

4) MgSO₄—10 mM
1.2 g of MgSO₄ can be dissolved in 1000 ml distilled water and sterilized by autoclaving.

5) Maltose 20% (w/v)
200 g of maltose can be dissolved in 1000 ml distilled water, and sterilized by filtration through a 20 μm filter.

6) MgSO₄—1 M
120.37 g of MgSO₄ can be dissolved in 1000 ml distilled water and sterilized by autoclaving.

7) LB with 10 mM of MgSO₄ and 0.2% of maltose
100 μl of MgSO₄ 1M and 100 μl of maltose 20% can be added to 99.8 ml of LB medium.

8) SM Buffer (Phage Storage Buffer)
5.8 g of NaCl, 2 g of MgSO₄, 50 ml of 1M Tris Hydrochloric acid (pH 7.5), 5 ml of 2% (w/v) gelatin can be dissolved in distilled water, to a final volume of 1000 ml, and then, sterilized by autoclaving.

9) Bacterial Strain (Host)
E. coli XL1 Blue MRA (Stratagene).

10) Phage:
λ GEM 11 (Promega).

11) Bacterial Cultivation on LB Plates
XL1 cells can be dispersed on the LB plate with a bacteriological loop according to a common procedure of bacterial inoculation. The plates can be incubated at 37° C. for 16 hours.

12) Bacterial Cultivation in LB Liquid Medium
A single colony of XL1 cells can be picked from an LB plate and can be inoculated in LB liquid medium with subsequent incubation at 37° C. for 16 hours (overnight), with shaking at 200 rpm.

13) Infection of the Host Bacterial Strain by the Phage
XL1 cells can be inoculated into the LB medium supplemented with 10 mM of MgSO₄ and 0.2% of maltose. Incubation at 37° C. with shaking at 200 rpm continued, until turbidity of 0.6 at a wavelength of 600 nm is achieved (estimated 4-5 hours). The grown culture can be centrifuged at 4000 rpm for 5 minutes. Supernatant can be discarded, and the bacteria can be re-suspended into the 10 mM of MgSO₄, until turbidity of 0.6 at wavelength of 600 nm is achieved. A required volume of SM buffer containing the phages can be added to 200 ml of the re-suspended bacteria. After incubation at 37° C. for 15 minutes two alternative procedures can be carried out:

(i) For lysate preparation an appropriate volume of LB medium can be added to the host-phage mixture, and incubated at 37° C. for 16 hours (overnight), with shaking at 200 rpm.

(ii) For phage appearance on solid medium (plaques), a molten Top Agarose (50° C.) can be poured on the host-phage mixture and quickly mixed and spread on the pre-warmed LB plate. After agarose solidification, incubation can be performed at 37° C. for 16 hours (overnight).

14) Extraction of the Phage DNA
Bacterial lysates can be centrifuged at 6000 rpm for 5-10 minutes for sedimentation of the bacterial debris. Supernatant can be collected and centrifuged at 14000 rpm for 30 minutes for sedimentation of the phage particles. Supernatant can be discarded and the phage pellet was re-suspended in SM buffer without gelatin. A mixture of nucleases (RNase and DNase from any supplier) can be added to the re-suspended phage for a final concentration of 5-10 Weiss units per 1 μl of the phage suspension. After an incubation of 30 minutes at 37° C. the DNA of the phage can be extracted by the following procedure:

(i) extraction with phenol: chloroform: iso-amil-alcohol (25:24:1 v/v);

(ii) removing of phenol contamination by chloroform;

(iii) precipitation to final concentration of 0.3 M Potassium Acetate and one volume of iso-propanol;

(iv) washing with 70% ethanol; and

(v) drying and re-suspension in distilled water for further analysis.

An increase in PFU at low phage dilutions (10⁻³ and 10⁻⁴) compared to the control can indicate that that the enriched nanostructure composition of the present embodiments affects the phage's ability to infect their hosts, and that the enriched nanostructure composition of the present embodiments increases the affinity between bacterial receptors and phage particles.

### Example 11

### Adherence to Microliter Plate

This Example describes a prophetic experiment for investigating the effect of the enriched nanostructure composition of the present embodiments on the adherence of coagulase-negative staphylococci to microtiter plate.

Production of slime polysaccharide, is crucial to biofilm generation and maintenance, and plays a major part as a virulence factor in bacteria [Gotz F., “Staphylococcus and biofilms,” Mol Microbiol 2002, 43(6):1367-78]. The slime facilitates adherence of bacteria to a surface and their accumulation to form multi-layered clusters. Slime also protects against the host's immune defense and antibiotic treatment [Kolari M. et al., “Colored moderately thermophilic bacteria in paper-machine biofilms,” to appear in J Ind Microbiol Biotechnol 2003]. Biofilm produced by bacteria can cause problems also in industry.

Most of current concepts for the prevention of slime are associated with search for new anti-infective active in biofilm and new biocompatible materials that complicate biofilm.

It has been demonstrated [Besnier J M et al., “Effect of subinhibitory concentrations of antimicrobial agents on adherence to silicone and hydrophobicity of coagulase-negative staphylococci,” Clin Microbiol Infect 1996, 1(4):244-248] that the adherence of coagulase-negative staphylococci onto silicone can be modified by sub-MICs of antimicrobial agents. This effect was different in the slime-producing and non-slime-producing strains, and was not correlated with the mechanism of the inhibitory effect of these antimicrobial agents, or the modification of hydrophobicity suggesting that some surface components, not involved in hydrophobicity, could play a role in vitro adherence.

The bacterial resistance of Staphylococcus epidermidis, a serious pathogen of implant-related infections, to antibiotics is related to the production of a glycocalyx slime that impairs antibiotic access and the killing by host defense mechanisms [Konig D P et al., “In vitro adherence and accumulation of Staphylococcus epidermidis RP 62 A and Staphylococcus epidermidis M7 on four different bone cements,” Langenbecks Arch Surg 2001, 386(5):328-32]. In vitro studies of different bone cements containing antibiotics, developed for the prevention of biomaterial-associated infection, could not always demonstrate complete eradication of biomaterial-adherent bacteria. Further efforts are done to find better protection from slime adherence.

In addition, surface interaction can modify slime adherence. For example, Farooq et al. [Farooq M et al., “Gelatin-sealed polyester resists Staphylococcus epidermidis biofilm infection,” J Surg Res 1999, 87(1):57-61] demonstrated that gelatin-impregnated polyester grafts inhibit Staphylococcus epidermidis biofilm infection in a canine model of aortic graft interposition. Gelatin-impregnated polyester grafts demonstrated in vivo resistance to coagulase-negative staphylococcal biofilm infection.

The objectives of the prophetic experiments in this example is to investigate the effect of the enriched nanostructure composition of the present embodiments on the adherence to plastic of a slime-producing Staphylococcus epidermidis.

Slime adherence can be quantitatively examined with a spectrophotometer optical density (OD) technique, as follows. Overnight cultures in TSB with the enriched nanostructure composition of the present embodiments and with regular water can be diluted 1:2.5 with corresponding media and placed in sterile micro titer tissue culture plates in a total volume of 250 μl each and incubated at 37° C. The plates can be rinsed 3 times with tap water, stained with crystal violet, and rinsed 3 more times with tap water. After drying, the OD of the stained adherent bacterial films can be measured with a MicroElisa Auto reader using wavelength of 550 nm. OD of bacterial culture can be measured before each staining using dual filter of 450 nm and 630 nm. The test of each bacterial strain can be performed in quadruplicates.

The experiment can be designed to evaluate slime adherence at intervals. The time table for the kinetics assessment can be 18, 20, 22, 24 and 43 hours. Strains were evaluated on the same plate. The enriched nanostructure composition of the present embodiments can be used for standard media preparation and can undergo standard autoclave sterilization.

Adherence values can be compared using ANOVA with repeated measurements for the same plate examination; grouping factors were plate and strain. A three-way ANOVA can be used for the different plate examination using SPSS™ 11.0 for Microsoft Windows™.

Difference in adherence, with higher adherence in the presence of the enriched nanostructure composition of the present embodiments compared to control can indicate a new order introduced by the nanostructures, leading to a change in water hydrophobic ability.

### Example 12

### Bacterial Colonies Growth

Colony growth of Bacillus subtilis can be investigated in the presence of the enriched nanostructure composition of the present embodiments. The control group can include the same bacteria in the presence water, purified by reverse osmosis (RO). Acceleration of colony growth in the presence of the enriched nanostructure composition of the present embodiments is expected.

### Example 13

### Macromolecule Binding to Solid Phase Matrix

A myriad of biological treatments and reactions can be performed on solid phase matrices such as Microtitration plates, membranes, beads, chips and the like. Solid phase matrices may have different physical and chemical properties, including, for example, hydrophobic properties, hydrophilic properties, electrical (e.g., charged, polar) properties and affinity properties.

The objectives of the present prophetic experiment is to investigate the effect of the enriched nanostructure composition of the present embodiments on the binding of biological material to microtitration plates and membranes having different physical and chemical properties.

Several types of microtitration plates can be used (e.g., MaxiSorp™, which contains mixed hydrophilic/hydrophobic regions and is characterized by high binding capacity of and affinity for IgG and other molecules; PolySorp™, which has a hydrophobic surface and is characterized by high binding capacity of and affinity for lipids; MedimSorp™, which has a surface chemistry between PolySorp™ and MaxiSorp™, and is characterized by high binding capacity of and affinity for proteins; Non-Sorp™, which is a non-treated microtitration plate characterized by low binding capacity of and affinity for biomolecules; MultiSor™, which has a hydrophilic surface and is characterized by high binding capacity of and affinity for Glycans; a medium binding microtitration plate, which has a hydrophilic surface and a binding capacity to IgG of 250 ng/cm²; a carbon binding microtitration plate, which covalently couples to carbohydrates; a high binding microtitration plate, which has a high adsorption capacity; and a high binding black microtitration plate, also having high adsorption capacity).

The binding efficiency of bio-molecules to the microtitration plates can be tested in four categories: ionic strengths, buffer pH, temperature and time.

The binding experiments can be conducted by coating the microtitration plate with fluorescent-labeled bio-molecules or with a mixture of labeled and non-labeled bio-molecules of the same type, removal of the non-bound molecules by washing and measuring the fluorescent signal remaining on the plate.

The following protocol can be employed:

1) Pre-diluting the fluorescent labeled bio-molecules to different concentrations (typically 0.4-0.02 μg/ml) in a binding buffer. Each set of dilutions can be performed in two binding buffers: (i) the enriched nanostructure composition of the present embodiments; and (ii) control water, purified by reverse osmosis.

2) Dispensing (in triplicates) 100 μl samples from each concentration to the microtitration plates, and measuring the initial fluorescence level.

3) Incubating the plates overnight at 4° C. or 2 hours at 37° C.

4) Discarding the coating solution.

5) Adding 150 μl of washing solution to each well and agitating at room temperature for 5 minutes. This washing step can be repeated three times. Typical washing solution includes 1×PBS, pH 7.4; 0.05% Tween20™; and 0.06 M NaCl.

6) Adding 200 μl fluorescence reading solution including 0.01 M Sodium hydroxide and incubating for 180 minutes or overnight at room temperature.

7) Reading the fluorescence using a fluorescence bottom mode, with excitation wavelength of 485 nm, emission wavelength of 535 and optimal gain of 10 flashes.

The effect of the enriched nanostructure composition of the present embodiments on the biding efficiency of glycoproteins (IgG of 150,000 D either labeled with Fluorescein isothiocyanate (FITX) or non-labeled) to the above described plates can be investigated. IgG is a polyclonal antibody composed of a mixture of mainly hydrophilic molecules. The molecules have a carbohydrate hydrophilic region, at the universal region and are slightly hydrophobic at the variable region. Such types of molecules are known to bind to MaxiSorp™ plates with very high efficiency (650 ng/cm²). It is expected that the enriched nanostructure composition of the present embodiments will enhance the binding efficiency of IgG.

The effect of the enriched nanostructure composition of the present embodiments on the binding efficiency of Peanut (Arachis hypogaea) agglutinin (PNA) can be investigated on the MaxiSorp™ and Non-Sorp™ plates. PNA is a 110,000 Dalton lectin, composed of four identical glycoprotein subunits of approximately 27,000 Daltons each. PNA lectin binds glycoproteins and glycolipids with a specific configuration of sugar residues through hydrophilic regions. PNA also possesses hydrophobic regions. The assay can include the use of three coating buffers: (i) carbonate buffer, pH 9.6, (ii) acetate buffer, pH 4.6 and (iii) phosphate buffer, pH 7.4. It is expected that the enriched nanostructure composition of the present embodiments will inhibit the binding of PNA.

The effect of the enriched nanostructure composition of the present embodiments on binding efficiency of nucleic acid can be investigated on the MaxiSorp™, Polysorp™ and Non-Sorp™ plates. Generally, DNA molecules do not bind well to polystyrene plates. Even more problematic is the binding of oligonucleotides, which are small single stranded DNA molecules, having a molecular weight of several thousand Daltons. It is expected that the enriched nanostructure composition of the present embodiments will enhance binding efficiency with and without the addition of salt to the coating buffer.

### Example 14

### Isolation and Purification of DNA

The effect of enriched nanostructure composition of the present embodiments on the purification of the PCR product can be studied by reconstitution of a PCR kit. It is expected that the use of enriched nanostructure composition of the present embodiments will improve the efficiency of the nucleic acid amplification process. In the following prophetic experiment, reconstitution of a Promega kit “Wizard—PCR preps DNA purification system” (A7170) is described.

The use of Promega Wizard™ kit involves the following steps:

1) Mix the purification buffer with the PCR sample to create conditions for binding the DNA to the Resin;

2) Mix the Resin suspension with the PCR mixture, for binding the DNA to the Resin, applies the resin samples to syringes and generate vacuum;

3) Add Isopropanol and suck the solution by vacuum to remove non bound DNA;

4) Elute the bound DNA with water; and

5) Performing gel electrophoresis as further detailed hereinbelow.

Reconstitution of the kit can be performed with the original water supplied with the kit (hereinafter control) or by replacing aqua solutions of the kit with either RO water or the enriched nanostructure composition of the present embodiments for steps 1, 2 and 4. In step 3 the identical 80% isopropanol solution as found in the kit can be used in all experiments.

The following protocol can be used for gel electrophoresis:

(a) Gel solution: 8% PAGE (+Urea) can be prepared with either RO water or the enriched nanostructure composition of the present embodiments;

(b) Add polymerization reagents containing 405 μl 10% APS and 55 μl TEMED (Sigma T-7024) to 50 ml of gel solution;

(c) Pour the gel solution into the gel cassette (Rhenium Ltd, Novex NC2015, 09-01505-C2), place the plastic combs and allow to polymerize for 30 minutes at room temperature;

(d) Remove the combs and strip off tape to allow assembling of two gels on two opposite sides of a single device;

(e) Fill in the inner chamber to the top of the gel and the outer chamber to about fifth of the gel height with running buffer-TBE ×1 in either RO water or the enriched nanostructure composition of the present embodiments;

(f) Prepare samples by diluting them in sample buffer containing TBE Ficoll, Bromophenol blue and urea (SBU), and mix 1:1 with the DNA sample;

(g) Load 8-10 μl of the mix into each well; and

(h) Set the power supply to 100 V and let the DNA migrate continue until the color dye (Bromophenol blue) reaches 1 cm from the bottom.

The following protocol can be used for gel staining visualization photographing and analyzing:

(a) Place the gels in staining solution containing 1 U/μl GelStar™ in 1×TBE for 15 minutes whilst shaking;

(b) Destain the gels for 30 minutes in 1×TBE buffer;

(c) Place the gels on U.V. table; use 365 nm light so as to see the DNA; and

(d) Using DC120™ digital camera, photograph the gels and store the digital information for further analysis.

PCR can be prepared from Human DNA using ApoE gene specific primers (fragment size 265 bp), according to the following protocol (for 100 reactions):

(a) Mark 0.2 μl PCR-tubes according to the appropriate serial number;

(b) Add 2.5 μl of 40 μg/ml Human DNA (Promega G 3041) or water to the relevant tubes;

(c) Adjust to 17 μl with 14.5 μl DDW;

(d) Prepare 3630 μl of the PCR mix;

(e) Add 33 μl of the mix to each tube;

(f) Place the samples in the PCR machine;

(g) Run a PCR program;

(h) Analyze 5 μl of each product on 8% PAGE gel; and

(i) Store reactions at −20° C.

### Example 15

### Column Capacity

This Example describes a prophetic experiment for investigating the effect of the enriched nanostructure composition of the present embodiments on column capacity. A plurality of (e.g., 100 or more) PCR reactions, each according to the protocols of Example 14 can be prepared and combined to make a 5 ml stock solution. The experiment can included two steps, in which in a preliminary step (hereinafter step A) can be directed at examining the effect of volume applied to the columns on binding and elution, and a primary step (hereinafter step B) can be directed at investigating the effect of the enriched nanostructure composition of the present embodiments on the column capacity.

In Step A, four columns (e.g., columns 1-4) can be applied with 50, 150, 300 or 600 μl stock PCR product solution, and 13 columns (e.g., 5-17) can be applied with 300 μl of stock PCR solution. All columns can be eluted with 50 μl of water. The eluted solutions can be loaded in lanes 7-10 in the following order: lane 7 (original PCR, concentration factor ×1), lane 8 (original ×3), lane 9 (×6) and lane 10 (×12). A “mix” of all elutions from columns 5-17 (×6) can be loaded in lane 11. Lanes 1-5 can be loaded with elutions from columns 1-4 and the “mix” of columns 5-17, pre-diluted to the original concentration (×1). Lane 6 can be the ladder marker.

The following protocol can be employed in Step A:

1) Mark the Wizard™ minicolumn and the syringe for each sample, and insert into the Vacuum Manifold;

2) Dispense 100 μl of each direct PCR purification buffer solution into a micro-tube;

3) Vortex briefly;

4) Add 1 ml of each resin solution and vortex briefly 3 times for 1 minute;

5) Add the Resin/DNA mix to the syringe and apply vacuum;

6) Wash by adding 2 ml of 80% isopropanol solution to each syringe and apply vacuum;

7) Dry the resin by maintaining the vacuum for 30 seconds;

8) Transfer the minicolumn to a 1.5 ml microcentrifuge tube;

9) Centrifuge at 10000 g for 2 minutes;

10) Transfer the minicolumn to a clean 1.5 ml tube;

11) Add 50 μl of the relevant water (nuclease free or the enriched nanostructure composition of the present embodiments);

12) Centrifuge at 10000 g for 20 second;

13) Transfer to 50 μl storage microtube and store at −20° C.;

14) Repeat steps 9-11 for a second elution cycle;

Visualization Steps:

15) Mix 6 μl of each sample with 6 μl loading buffer;

16) Load 10 μl of each mix in acrylamide urea gel (AAU) and run the gel at 70 V 10 mAmp;

17) Stain the gel with Gel Star™ solution (5 μl of 10000 u solution in 50 ml TBE), shake for 15 minutes at room temperature;

18) Shake in TBE buffer at room temperature for 30 minutes to destain the gel; and

19) photograph the gel.

In Step B the “mixed” elution of Step A can be used as “concentrated PCR solution” and applied to 12 columns. Columns 1-5 can be applied with 8.3 μl, 25 μl, 50 μl, 75 μl and 100 μl respectively using the kit reagents. The columns can be eluted by 50 μl kit water and 5 μl of each elution can be applied to the corresponding lane on the gel. Columns 7-11 can be treated as column 1-5 but with the enriched nanostructure composition of the present embodiments as binding and elution buffers. The samples can be applied to the corresponding gel lanes. Column 13 can be serve as a control with the “mix” of columns 5-17 of Step A.

The following protocol can be employed in Step B:

1) Mark the Wizard™ minicolumn and syringe to be used for each sample and insert into the vacuum manifold;

2) Dispense 100 μl of each direct PCR purification buffer solution into micro-tube;

3) Vortex briefly;

4) Add 1 ml of each resin solution and vortex briefly 3 times for 1 minute;

5) Add the Resin/DNA mix to the syringe and apply vacuum;

6) Wash by adding 2 ml of 80% isopropanol solution to each syringe and apply vacuum;

7) Dry the resin by continuing to apply the vacuum for 30 seconds.

8) Transfer the minicolumn to 1.5 ml microcentrifuge tube.

9) Centrifuge at 10000 g for 2 minutes.

10) Transfer the minicolumn to a clean 1.5 ml tube.

11) Add 50 μl of nuclease free or the enriched nanostructure composition of the present embodiments.

12) Centrifuge at 10000 g for 20 seconds.

13) Transfer to a 50 μl storage micro-tube and store at −20° C.

14) Repeat steps 11-13 for a second elution cycle.

Visualization steps can be the same as in Step A.

Higher intensity in lanes 7-11 compared to lanes 1-5 and 7-11 can indicate that the enriched nanostructure composition of the present embodiments is capable of binding more DNA than the kit reagents.

### Example 16

### Isolation of DNA by Gel Electrophoresis

Gel Electrophoresis is a routinely used method for determination and isolation of DNA molecules based on size and shape. DNA samples are applied to an upper part of the gel, serving as a running buffer surrounding the DNA molecules. The gel is positively charged and forces the negatively charged DNA fragments to move downstream the gel when electric current is applied. The migration rate is faster for smaller and coiled or folded molecules and slower for large and unfolded molecules. Once the migration is completed, DNA can be tagged by fluorescent label and is visualized under UV illumination. The DNA can be also transferred to a membrane and visualized by enzymatic coloration at high sensitivity. DNA is evaluated according to its position on the gel and the band intensity.

Following is a description of prophetic experiments for investigating the effect of the enriched nanostructure composition of the present embodiments on DNA migration by gel electrophoresis.

Two types of DNA can be used: (i) PCR product, 280 base pair; and (ii) ladder DNA composed of eleven DNA fragments of the following sizes: 80, 100, 200, 300, 400, 500, 600, 700, 800, 900 and 1030 bp. The gel can be prepared according to the protocols of Example 14.

It is expected that the migration speed in the presence of RO water will be higher than the migration speed in the presence of the enriched nanostructure composition of the present embodiments. It is expected that the enriched nanostructure composition of the present embodiments will cause the retardation of DNA migration as compared to RO water. Such results can indicate that under the influence of the enriched nanostructure composition of the present embodiments, the DNA configuration is changed in a manner such that the folding of the DNA is decreased (un-folding). The un-folding of DNA in the enriched nanostructure composition of the present embodiments may indicate that stronger hydrogen boned interactions exists between the DNA molecule and the enriched nanostructure composition of the present embodiments in comparison to RO water.

### Example 17

### Enzyme Activity and Stability

Increasing both enzyme activity and stability are important for enhancing efficiency and reducing costs of any process utilizing enzymes. During long term storage, prolonged activity and also when over-diluted, enzymes are typically exposed to stress which may contribute to loss of stability and ultimately to loss of activity.

This Example describes a prophetic experiment for investigating the effect of the enriched nanostructure composition of the present embodiments on the activity and stability of enzymes. This prophetic study relates to two commonly used enzymes in the biotechnological industry: Alkaline Phosphatase (AP), and β-Galactosidase. Two forms of AP can be used: an unbound form and a bound form in which AP was bound to Strept-Avidin (ST-AP).

Following is a description of prophetic experiments in which the effect of the enriched nanostructure composition of the present embodiments on diluted enzymes can be investigated. The dilutions can be performed either in RO water or in the enriched nanostructure composition of the present embodiments without additives and in neutral pH (7.4).

### Unbound Form of Alkaline Phosphatase

Alkaline Phosphatase (Jackson INC) can be serially diluted in either RO water or the enriched nanostructure composition of the present embodiments. Diluted samples 1:1,000 and 1:10,000 can be incubated in tubes at room temperature.

At different time intervals, enzyme activity can be determined by mixing 10 μl of enzyme with 90 μl pNPP solution (AP specific colorimetric substrate). The assay can be performed in microtitration plates (4 or more repeats for each test point). Color intensity can be determined by an ELISA reader at wavelength of 405 nm.

Enzyme activity can be determined at time t=0 for each dilution, both in RO water and in the enriched nanostructure composition of the present embodiments. Stability can be determined as the activity after 22 hours (t=22) and 48 hours (t=48) divided by the activity at t=0.

It is expected that the activity in the presence of the enriched nanostructure composition of the present embodiments will be higher that the activity in the presence of RO water.

### Bound Form of Alkaline Phosphatase

Binding an enzyme to another molecule typically increases its stability. Enzymes are typically stored at high concentrations, and only diluted prior to use to the desired dilution. Following is a description of prophetic experiments for investigating the stabilization effect of the enriched nanostructure composition of the present embodiments on enzymes which are stored at high concentrations for prolonged periods of time.

Strept-Avidin Alkaline Phosphatase (Sigma) can be diluted 1:10 and 1:10,000 in RO water and in the enriched nanostructure composition of the present embodiments. The diluted samples can be incubated in tubes for 5 days at room temperature. All samples can be diluted to a final enzyme concentration of 1:10,000 and the activity can be determined as further detailed hereinabove. Enzyme activity can be determined at time t=0 and after 5 days.

It is expected that the enzyme will be substantially more active in the enriched nanostructure composition of the present embodiments than in RO water.

### β-Galactosidase

Experiments with β-Galactosidase can be performed according to the same protocol used for the Alkaline Phosphatase prophetic experiments described above with the exception of enzyme type, concentration and in incubation time. β-Galactosidase (Sigma) can be serially diluted in RO water and in the enriched nanostructure composition of the present embodiments. The samples can be diluted to 1:330 and 1:1000 and can be incubated at room temperature.

The enzyme activity can be determined at time intervals 0, 24 hours, 48 hours, 72 hours and 120 hours, by mixing 10 μl of enzyme with 100 μl of ONPG solution (β-Gal specific colorimetric substrate) for 15 minutes at 37° C. and adding 50 μl stop solution (1M Na₂Hco₃). The assay can be performed in microtitration plates (about 8 repetitions from each test point). An ELISA reader at wavelength of 405 nm can be used to determine color intensity.

The enzyme activity can be determined at time t=0 for each dilution, for the RO water and for the enriched nanostructure composition of the present embodiments. It is expected that the activity in the presence of the enriched nanostructure composition of the present embodiments will be higher than the activity in the presence of RO water.

### Activity and Stability of Dry Alkaline Phosphatase

Many enzymes are dried before storage. The drying process and the subsequent storage in a dry state for a prolonged period of time are known to effect enzyme activity. dilution. Following is a description of prophetic experiments for investigating the stabilization effect of the enriched nanostructure composition of the present embodiments on the activity and stability of dry alkaline phosphatase.

Alkaline Phosphatase (Jackson INC) can be diluted 1:5000 in RO water and in the enriched nanostructure composition of the present embodiments as further detailed hereinabove.

Several (e.g., 9) microtitration plates can be filled with aliquots of 5 μl solution. One plate can be tested for enzyme activity at time t=0, as further detailed hereinabove, and the remaining plates can be dried at 37° C. overnight. The drying process can be performed in a desiccated environment for 16 hours.

Two of the plates can be tested for enzyme activity by initial cooling to room temperature and subsequent addition of 100 μl pNPP solution at room temperature. Color intensity can be determined by an ELISA reader at a wavelength of 405 nm and the stability can be calculated as further detailed hereinabove. The other plates can be transferred to 60° C. for 30 minutes and the enzyme activity can be determined thereafter. The purpose of the drying and heat treatments is to damage the enzyme. It is expected that the enriched nanostructure composition of the present embodiments will at least partially stabilize the activity of the enzyme.

### Example 18

### Anchoring of DNA

This Example describes a prophetic experiment for investigating the effect of anchoring DNA with glass beads in the presence or absence of the enriched nanostructure composition of the present embodiments.

The enriched nanostructure composition of the present embodiments is interchangeable referred to in this example as “gas enriched nanostructure composition” abbreviated “GENC.”

Anchoring polynucleotides to a solid support such as glass beads can be of utmost benefit in the field of molecular biology research and medicine. Typically, DNA manipulations comprise a sequence of reactions, one following the other, including PCR, ligation, restriction and transformation. Each reaction is preferably performed under its own suitable reaction conditions requiring its own specific buffer. Typically, in between each reaction, the DNA or RNA sample must be precipitated and then reconstituted in its new appropriate buffer. Repeated precipitations and reconstitutions takes time and more importantly leads to loss of starting material, which can be of utmost relevance when this material is rare.

Following is a description of prophetic experiments for investigating what effect the enriched nanostructure composition of the present embodiments has on DNA in the presence of glass beads during a PCR reaction.

PCR can be prepared from a pBS plasmid cloned with a 750 base pair gene using a T7 forward primer (TAATACGACTCACTATAGGG) SEQ ID NO:1 and an M13 reverse primer (GGAAACAGCTATGACCATGA) SEQ ID NO:2 such that the fragment size obtained is 750 bp. The primers can be constituted in PCR-grade water at a concentration of 200 μM (200 pmol/μl). These can be subsequently diluted 1:20 in the enriched nanostructure composition of the present embodiments to a working concentration of 10 μM each to make a combined mix. For example 1 μl of each primer (from 200 μM stock) can be combined and diluted with 18 μl of the enriched nanostructure composition of the present embodiments mixed and spun down. The concentrated DNA can be diluted 1:500 with the enriched nanostructure composition of the present embodiments to a working concentration of 2 ρg/μl. The PCR can be performed in a Biometra T-Gradient PCR machine. The enzyme can be SAWADY Taq DNA Polymerase (PeqLab 01-1020) in buffer Y.

A PCR mix can be prepared as follows:

| Final conc | X1 | | Concentration | Reagents |
|---|---|---|---|---|
| X1 | 1 | μl | X10 | Buffer Y |
| 0.2 mM | 0.2 | μl | 10 mM each | dNTPs |
| 0.4 units | 0.08 | μl | 5 u/μl | Taq |
| | 3.22 | μl | | GENC |

| | |
|---|---|
| Pick a few beads with a tip end and gently tap on the | Glass Beads - |
| tip on top of an open tube - a few glass beads will | without |
| fall into the tube. The amount of the powder in the | any treatment. |
| mix is preferably almost invisible, since too much | |
| glass powder may inhibit the PCR reaction | |

The samples can be mixed preferably without forming vortex. They can be placed in a PCR machine at 94° C. for 1 min and then removed. 4.5 μl of the PCR mix can be then aliquoted into clean tubes to which 0.5 μl of primer mix and 5 μl of diluted DNA can be added in that order. After mixing the samples can be placed in the PCR machine and the following PCR program can be used

| Time | Temp | Step |
|---|---|---|
| 10 sec | 94° C. | Step 1 |
| 10 sec | 50° C. | Step 2 |
| 10 sec | 74° C. | Step 3 |

The products of the PCR reaction can be run on 8% PAGE gels for analysis as described hereinabove.

It is expected that the enriched nanostructure composition of the present embodiments will be required during the PCR reaction in the presence of glass beads for the PCR product to be visualized.

### Example 19

### Real-Time PCR

The detection and quantification of DNA and cDNA nucleic acid sequences is of importance for a wide range of applications including forensic science, medicine, drug development and molecular biology research. Real-time PCR monitors the fluorescence emitted during a PCR reaction as an indicator of amplicon production during each PCR cycle (i.e. in real time) as opposed to the endpoint detection of conventional PCR which relies on visualization of ethidium bromide in agarose gels.

Due to its high sensitivity, real-time PCR is particularly relevant for detecting and quantifying very small amounts of DNA or cDNA. Improving sensitivity and reproducibility and decreasing the reaction volumes required for real-time PCR would aid in conserving precious samples.

This Example describes a prophetic experiment for investigating the effect of the enriched nanostructure composition of the present embodiments on the sensitivity and reaction volumes of real-time PCR reactions.

The enriched nanostructure composition of the present embodiments is interchangeable referred to in this example as “gas enriched nanostructure composition” abbreviated “GENC.”

### Sensitivity Testing

Real-time PCR reactions can be performed using SYBR Green method on Applied Biosystem 7300 PCR System. Reactions can be performed on 96 well plates (Corning, N.Y.). Primer sequences can be as follows:

| | | |
|---|---|---|
| Forward primer: | CACCAGACTGACTCCTCATT | SEQ ID NO: 3 |
| Reverse primer: | CCTGTTGCTGCACATATTCC | SEQ ID NO: 4 |

Two sets of 12 samples each can be prepared as detailed hereinbelow, one with nuclease-free water and the other with the enriched nanostructure composition of the present embodiments. For each set a 13× mix can be prepared. The sample can be prepared according to the following protocol

| Component | μl/well | Pool per 13 reactions (μl) |
|---|---|---|
| Forward primer | 0.5 | 6.5 |
| Reverse primer | 0.5 | 6.5 |
| ABI SYBR green mix | 10 | 130 |
| Water or GENC | 6 | 78 |

The cDNA sample can be diluted in water or GENC in serial dilutions starting from 1:5 and ending with 1:2560 (10 dilutions in total). The 1:5 dilution can be prepared using 3 μl of the original cDNA+12 μl H₂O or the GENC. The dilutions which followed were prepared by taking 7.5 μl of sample and 7.5 μl of H₂O or GENC.

17 μl of the mix can be added to 3 μl of cDNA sample. The first reaction in each set can be an undiluted cDNA sample.

A standard curve can be plotted of the number of PCR cycles needed for the fluorescence to exceed a chosen level (threshold cycle (Cₜ)) versus their corresponding Log cDNA concentrations for the diluted samples. This standard curve is a measure of the linearity of the process, the reaction efficiency.

A dissociation curve can be plotted for the reactions of each standard curve for the diluted samples.

Both standard and dissociation curves can be plotted using an automatic baseline determination. Standard curves only can be plotted at a manual background cut-off of 0.2 and following removal of identical or non-identical outlier values from each set.

It is expected that for the enriched nanostructure composition of the present embodiments there will be higher regression value compared to water. Such results can indicate that the presence of the enriched nanostructure composition of the present embodiments provides a more accurate assessment of quantity for a wider dynamic range of concentrations. It is further expected that the dynamic range and efficiency of amplification will be higher in the presence of the enriched nanostructure composition of the present embodiments.

### Volume Testing

Following is a description of a prophetic experiment for examining the possibility that execution of real-time PCR reactions using the enriched nanostructure composition of the present embodiments instead of water would enable lower reaction volumes while retaining sensitivity.

All materials can be identical to those used above for determining sensitivity. The cDNA samples can be diluted 1:80.

The following reaction volumes can be tested: 5 ul, 10 ul and 15 ul. Each of the three volume sets can include a strip of 8 reactions: triplicates of reactions with and without GENC and one negative control (minus template). In addition to decreased reaction volumes the ratio between the SYBR green solution and the solvent (either water or GENC) can be changed as further detailed below.

| | standard | 20 μl | 5 μl vol. | 10 μl vol. | 15 μl vol. |
|---|---|---|---|---|---|
| | 20 μl | reaction | test | test | test |
| Component | reaction | for vol. test | pool 30 μl | pool 60 μl | Pool 80 μl |
| Forward | 0.5 | 0.5 | 0.75 | 1.5 | 2 |
| primer | | | | | |
| Reverse | 0.5 | 0.5 | 0.75 | 1.5 | 2 |
| primer | | | | | |
| ABI SYBR | 10 | 5 | 7.5 | 15 | 20 |
| green mix | | | | | |
| water/GENC | 6 | 11 | 16.5 | 33 | 44 |
| cDNA | 3 | 3 | 4.5 | 9 | 12 |
| sample | | | | | |

Pools for each volume test can be prepared in water or in GENC as indicated and then aliquoted at the desired volume, to reaction wells. All results can be read at background cutoff value of 0.2.

It is expected that the reactions performed in the presence of the enriched nanostructure composition of the present embodiments will be more reproducible.

### Example 20

### Ultrasonic Tests

This Example describes a prophetic experiment for subjecting the enriched nanostructure composition of the present embodiments to a series of ultrasonic tests in an ultrasonic resonator.

Measurements of ultrasonic velocities in the enriched nanostructure composition of the present embodiments and double dest. water can be performed using a ResoScan® research system (Heidelberg, Germany).

### Calibration

Both cells of the ResoScan® research system can be filled with standard water supplemented with 0.005% Tween 20 and measured during an isothermal measurement at 20° C. The difference in ultrasonic velocity between both cells can be used as the zero value in the isothermal measurements as further detailed hereinbelow.

### Isothermal Measurements

Cell 1 of the ResoScan® research system can be used as reference and can be filled with dest. Water. Cell 2 can be filled with the enriched nanostructure composition of the present embodiments. Absolute Ultrasonic velocities can be measured at 20° C. In order to allow comparison of the experimental values, the ultrasonic velocities can be corrected to 20.000° C.

It is expected that the absolute ultrasonic velocity as measured for the enriched nanostructure composition of the present embodiments will be higher than the absolute ultrasonic velocity as measured for the dist. water.

### Example 21

### Hybridization of RNA to a Chip

This Example describes a prophetic experiment for investigating the strength of hybridization between RNA samples to a DNA chip in the presence and absence of the enriched nanostructure composition of the present embodiments.

A GEArray Q Series Human Signal Transduction PathwayFinder Gene Array: HS-008 can be used.

RNA can be extracted from human lymphocytes using Rneasy kit (QIAGEN). The RNA can be labeled using the GEArray AmpoLabeling-LPR Kit (Catalog Number L-03) according to the Manufacturers protocol.

Hybridization of the RNA sample to the array can be performed according to the Manufacturers protocol. Essentially the membrane can be pre-wet in deionized water for five minutes following which it can be incubated in pre-warmed GEAhyb Hybridization Solution (GEArray) for two hours at 60° C. Labelled RNA can be added to the hybridization solution and left to hybridize with the membrane overnight at 60° C. Following rinsing, the membrane can be exposed to an X ray film for autoradiography for a two second or ten second exposure time.

It is expected that hybridization will be increased in the presence of the enriched nanostructure composition of the present embodiments to a DNA chip. This can be demonstrated by observing the signal strength following identical exposure periods.

### Example F21

### Buffering Capacity

This Example describes a prophetic experiment for investigating the effect of the enriched nanostructure composition of the present embodiments on buffering capacity.

Sodium hydroxide and Hydrochloric acid can be added to either 50 ml of RO water or the enriched nanostructure composition of the present embodiments and the pH can be measured, according to the following protocol:

Sodium hydroxide titration: —add 1 μl to 15 μl of 1M Sodium hydroxide.

Hydrochloric acid titration: —add 1 μl to 15 μl of 1M Hydrochloric acid.

It is expected that for the enriched nanostructure composition of the present embodiments, greater amounts of Sodium hydroxide or Hydrochloric acid will be required in order to reach the same pH level that is needed for RO water. Such result can indicate that the enriched nanostructure composition of the present embodiments has buffering capacities.

### Example 22

### Solvent Capability

This Example describes various prophetic experiments for investigating the ability of the enriched nanostructure composition of the present embodiments to dissolve Daidzein-daunomycin conjugate (CD-Dau), Daunrubicine (Cerubidine hydrochloride), and t-boc derivative of daidzein (tboc-Daid), all of which are known not to dissolve in water.

The enriched nanostructure composition of the present embodiments is interchangeable referred to in this example as “gas enriched nanostructure composition” abbreviated “GENC.”

### Solubilizing CD-Dau

Required concentration: 3 mg/ml GENC.

Properties: The material dissolves in DMSO, acetone, acetonitrile.

Properties: The material dissolves in EtOH.

5 different glass vials can be prepared:

(i) 5 mg CD-Dau+1.2 ml GENC.

(ii) 1.8 mg CD-Dau+600 μl acetone.

(iii) 1.8 mg CD-Dau+1500 acetone+450 μl GENC (25% acetone).

(iv) 1.8 mg CD-Dau+600 μl 10% *PEG (Polyethylene Glycol).

(v) 1.8 mg CD-Dau+600 μl acetone+600 μl GENC.

The samples can be vortexed and spectrophotometer measurements can be performed. Vials (ii), (iii) and (v) can be were left opened in order to evaporate the acetone. Dissolvent of the CD-Dau can be compared to assess the capability of the enriched nanostructure composition of the present embodiments to dissolve CD-Dau.

### Solubilizing Daunorubicine (Cerubidine Hydrochloride)

Required concentration: 2 mg/ml

2 mg Daunorubicine+1 ml GENC can be prepared in one vial and 2 mg of Daunorubicine+1 ml RO water can be prepared in a second vial. It is expected that the material will be dissolved easily both in the enriched nanostructure composition of the present embodiments and in RO water.

Solubilizing t-boc

Required concentration: 4 mg/ml

1.14 ml of EtOH can be added to one glass vial containing 18.5 mg of t-boc (an oily material). This can then be divided into two vials and 1.74 ml GENC or RO water can be added to the vials such that the solution will comprise 25% EtOH. Following spectrophotometer measurements, the solvent can be evaporated from the solution and the enriched nanostructure composition of the present embodiments can be added to both vials to a final volume of 2.31 ml in each vial. The solutions in the two vials can be merged to one clean vial and packaged for shipment under vacuum conditions.

It is expected that following addition of the enriched nanostructure composition of the present embodiments and subsequent evaporation of the solvent with heat, the material will be dissolved in the enriched nanostructure composition of the present embodiments.

### Example 23

### Solvent Capability

This Example describes two prophetic experiments for investigating the ability of the enriched nanostructure composition of the present embodiments to dissolve two herbal materials—AG-14A and AG-14B, both of which are known not to dissolve in water at a concentration of 25 mg/ml.

### Part 1

2.5 mg of each material (AG-14A and AG-14B) can be diluted in either the enriched nanostructure composition of the present embodiments alone or a solution comprising 75% of the enriched nanostructure composition of the present embodiments and 25% ethanol, such that the final concentration of the powder in each of the four tubes will be 2.5 mg/ml. The tubes can be vortexed and heated to 50° C. so as to evaporate the ethanol.

It is expected that suspension of AG-14B in the enriched nanostructure composition of the present embodiments will not aggregate, contrary to RO water in which it aggregates. It is further expected that the enriched nanostructure composition of the present embodiments will not AG-14A and AG-14B.

### Part 2

5 mg of AG-14A and AG-14B can be diluted in 62.5 μl EtOH+187.5 μl of the enriched nanostructure composition of the present embodiments. A further 62.5 μl of the enriched nanostructure composition of the present embodiments can be added. The tubes can be vortexed and heated to 50° C. so as to evaporate the ethanol.

It is expected that suspension in EtOH prior to addition of the enriched nanostructure composition of the present embodiments and then evaporation thereof will dissolve AG-14A and AG-14B.

### Example 24

### Solvent Capability

This Example describes two prophetic experiments for investigating the ability of the enriched nanostructure composition of the present embodiments to dissolve cytotoxic peptides. In addition, This Example describes a prophetic experiment for measuring the effect of the peptides on Skov-3 cells in order to ascertain whether the enriched nanostructure composition of the present embodiments influenced the cytotoxic activity of the peptides.

The following peptides can be used: Peptide X, X-5FU, NLS-E, Palm-PFPSYK (CMFU), PFPSYKLRPG-NH₂, NLS-p2-LHRH and F-LH-RH-palm kGFPSK), all of which are known not to dissolve in water. All seven peptides can be dissolved in the enriched nanostructure composition of the present embodiments at 0.5 mM. Spectrophotometric measurements can then be taken.

Skov-3 cells can be grown in McCoy's 5A medium, and diluted to a concentration of 1500 cells per well, in a 96 well plate. After 24 hours, 2 μl (0.5 mM, 0.05 mM and 0.005 mM) of the peptide solutions can be diluted in 1 ml of McCoy's 5A medium, for final concentrations of 10⁻⁶ M, 10⁻⁷ M and 10⁻⁸ M respectively. Several repeats (e.g., 9) can be made for each treatment. Each plate can contain two peptides in three concentration, and 6 wells of control treatment. 90 μl of McCoy's 5A medium+peptides can be added to the cells. After 1 hour, 10 μl of FBS can be added (in order to prevent competition). Cells can be quantified after 24 and 48 hours in a viability assay based on crystal violet. This dye stains DNA. Upon solubilization, the amount of dye taken up by the monolayer can be quantified in a plate reader.

It is expected that the peptides will be diluted in the enriched nanostructure composition of the present embodiments and that the dissolved peptides will comprise cytotoxic activity.

### Example 25

### Solvent Capability

This Example describes two prophetic experiments for investigating the ability of the enriched nanostructure composition of the present embodiments to dissolve retinol.

The enriched nanostructure composition of the present embodiments is interchangeable referred to in this example as “gas enriched nanostructure composition” abbreviated “GENC.”

Retinol can be solubilized in GENC under the following conditions:

1% retinol (0.01 gr in 1 ml) in EtOH and GENC.

0.5% retinol (0.005 gr in 1 ml) in EtOH and GENC.

0.5% retinol (0.125 gr in 25 ml) in EtOH and GENC.

0.25% retinol (0.0625 gr in 25 ml) in EtOH and GENC. Final EtOH concentration: 1.5%

It is expected that the retinol will be solubilized easily in alkali enriched nanostructure composition rather than acidic enriched nanostructure composition.

### Example 26

### Solvent Capability

This Example describes prophetic experiments for investigating the ability of the enriched nanostructure composition of the present embodiments to dissolve material X at a final concentration of 40 mg/ml.

The enriched nanostructure composition of the present embodiments is interchangeable referred to in this example as “gas enriched nanostructure composition” abbreviated “GENC.”

### Part 1

This part can be directed to the solubility in GENC and DMSO. In a first test tube, 25 μl GENC can be added to 1 mg of material “X”. In a second test tube 25 μl of DMSO can be added to 1 mg of material “X”. Both test tubes can be vortexed and heated to 60° C. and shaken for 1 hour on a shaker. Dissolvent of material “X” in the vials can assessed and compared.

### Part 2

This part can be directed to the reduction of DMSO and examination of the material stability/kinetics in different solvents over the course of time.

6 different test tubes can be analyzed each containing a total reaction volume of 25 μl:

(i) 1 mg “X”+25 μl GENC (100%).

(ii) 1 mg “X”+12.5 μl DMSO+12.5 μl GENC (50%).

(iii) 1 mg “X”+12.5 μl DMSO+12.5 μl GENC (50%).

(iv) 1 mg “X”+6.25 μl DMSO+18.75 μl GENC (25%).

(v) 1 mg “X”+25 μl GENC+sucrose (10%). The latter being 0.1 g sucrose+1 ml GENC.

(vi) 1 mg+12.5 μl DMSO+12.5 μl of dehydrated enriched nanostructure composition according to various exemplary embodiments of the present invention (50%), the latter can be achieved by dehydration GENC for 90 min at 60° C.

All test tubes can be vortexed, heated to 60° C. and shaken for 1 hour.

Dissolvent of material “X” in the vials can assessed and compared.

### Part 3

This part can be directed to further reduction of DMSO and examination of the material stability/kinetics in different solvents over the course of time.

1 mg of material “X”+50 μl DMSO can be placed in a glass tube. 50 μl GENC can be titred (every few seconds 5 μl) into the tube, and then 500 μl of a solution GENC (9% DMSO+91% GENC) can be added.

In a second glass tube, 1 mg of material “X”+50 μl DMSO can be added. 50 μl of RO can be titred (every few seconds 5 μl) into the tube, and then 500 μl of a solution of RO (9% DMSO+91% RO) can be added.

Dissolvent of material “X” in the vials can assessed and compared.

### Example 27

### Solvent Capability

This Example describes prophetic experiments for investigating the ability of the enriched nanostructure composition of the present embodiments to dissolve SPL 2101 and SPL 5217 at a final concentration of 30 mg/ml.

SPL 2101 can be dissolved in its optimal solvent (ethanol) and SPL 5217 can be dissolved in its optimal solvent (acetone). The two compounds can be put in glass vials and can be kept in dark and cool environment. Evaporation of the solvent can be performed in a dessicator and over a long period of time the enriched nanostructure composition of the present embodiments can be added to the solution until all traces of the solvents are disappeared.

It is expected that the enriched nanostructure composition of the present embodiments will dissolve SPL 2101 and SPL 5217.

### Example 28

### Solvent Capability

This Example describes a prophetic experiment for investigating the ability of the enriched nanostructure composition of the present embodiments to dissolve Taxol (Paclitaxel) at a final concentration of 0.5 mM.

0.5 mM Taxol solution can be prepared (0.0017 gr in 4 ml) in either DMSO or the enriched nanostructure composition of the present embodiments with 17% EtOH. Absorbance can be detected with a spectrophotometer.

About 150,000 293T cells can be seeded in a 6 well plate with 3 ml of DMEM medium. Each treatment can be grown in DMEM medium based on RO or the enriched nanostructure composition of the present embodiments. Taxol (dissolved in the enriched nanostructure composition of the present embodiments or DMSO) can be added to final concentration of 1.666 μM (10 μl of 0.5 mM Taxol in 3 ml medium). The cells can be harvested following a 24 hour treatment with taxol and counted using trypan blue solution to detect dead cells.

It is expected that Taxol will be dissolved both in DMSO and the enriched nanostructure composition of the present embodiments and that the Taxol will comprise a cytotoxic effect following solution in the enriched nanostructure composition of the present embodiments.

### Example 29

### Solvent Capability

This Example describes another prophetic experiment for investigating the ability of the enriched nanostructure composition of the present embodiments to dissolve Taxol at a final concentration of 0.5 mM in the presence of 0.08% ethanol.

0.5 mM Taxol solution can be prepared (0.0017 gr in 4 ml). Taxol can be dissolved in ethanol and exchanged to the enriched nanostructure composition of the present embodiments using an RT slow solvent exchange procedure which can be extended until less than 40% ethanol remain in the solution. The solution can be sterilized using a 0.2 μm filter. Taxol can be separately prepared in DMSO (0.5 mM). Both solutions can be kept at −20° C. Absorbance can be detected with a spectrophotometer.

About 2000 PC3 cells can be seeded per well in a 96-well plate with 100 μl of RPMI based medium with 10% FCS. 24 hours post seeding, 2 μl, 1 μl and 0.5 μl of 0.5 mM taxol can be diluted in 1 ml of RPMI medium, reaching a final concentration of 1 μM, 0.5 μM and 0.25 μM respectively. Several (e.g., eight or more) replicates can be run per treatment. Cell proliferation can be assessed by quantifying the cell density using a crystal violet colorimetric assay 24 hours after the addition of taxol.

24 hours post treatment, the cells can be can behed with PBS and fixed with 4% paraformaldehyde. Crystal violet can be added and incubated at room temperature for 10 minutes. After washing the cells several (e.g., three) times, a solution with 100 M Sodium Citrate in 50% ethanol can be used to elute the color from the cells. Changes in optical density can be read at 570 nm using a spectrophotometric plate reader. Cell viability can be expressed as a percentage of the control optical density, deemed as 100%, after subtraction of the blank.

It is expected that Taxol will be dissolved in the enriched nanostructure composition of the present embodiments and that it will show similar in vitro cell viability/cytotoxicity on a human prostate cancer cell line as taxol dissolved in DMSO.

### Example 30

### Solvent Capability

This Example describes a prophetic experiment for investigating the ability of the enriched nanostructure composition of the present embodiments to dissolve insoluble Cephalosporin at a concentration of 3.6 mg/ml using a slow solvent exchange procedure, and for assessing its bioactivity on E. Coli DH5α strain transformed with the Ampicillin (Amp) resistant bearing pUC19 plasmid.

The enriched nanostructure composition of the present embodiments is interchangeable referred to in this example as “gas enriched nanostructure composition” abbreviated “GENC.”

25 mg of cephalosporin can be dissolved in 5 ml organic solvent Acetone (5 mg/ml). The procedure of exchanging the organic solvents with GENC can be performed on a multi block heater which can be set at 30° C., and inside a desiccator and a hood. Organic solvent concentration can be calculated according to the equations below:

Analytical Balance

% Acetone ml 1−0.1739X=Weighed value

% EtOH ml 1−0.2155X=Weighed value

Refractometer

% Acetone ml 0.0006X+1.3328=Refractive Index (RI) value

% EtOH ml 0.0006X+1.3327=Refractive Index (RI) value

The solution can be filtered successfully using a 0.45 μm filter. Spectrophotometer readouts of the solution can be performed before and after the filtration procedure.

DH5α E. Coli bearing the pUC19 plasmid (Ampicillin resistant) can be grown in liquid LB medium supplemented with 100 μg/ml ampicillin overnight at 37° C. and 220 Rounds per minute (rpm). 100 μL of the overnight (ON) starter can be re-inoculated in fresh liquid LB as follows:

(i) 3 tubes with 100 μl GENC: (only 2^{nd} experiment) and no antibiotics (both experiments).

(ii) 3 tubes with 10 μl of the Cephalosporin stock solution (50 ug/ml).

(iii) 3 tubes with 100 μl of the Cephalosporin stock solution (5 ug/ml).

Bacteria can be incubated at 37° C. and 220 rpm. Sequential OD readings can be taken every hour using a 96 wells transparent plate with a 590 nm filter using a TECAN SPECTRAFlour Plus.

It is expected that cephalosporin will be bioavailable and bioactive as a bacterial growth inhibitor even after substantial dilution in the enriched nanostructure composition of the present embodiments.

### Example 31

### Stabilizing Effect

This Example describes a prophetic experiment for investigating the effect of the enriched nanostructure composition of the present embodiments on the stability of a protein.

Commercially available Taq polymerase enzymes (e.g., Peq-lab and Bio-lab) can be checked in a PCR reaction to determine their activities in ddH₂O(RO) and the enriched nanostructure composition of the present embodiments. The enzyme can be heated to 95° C. for different periods of time, from one hour to 2.5 hours.

Two types of reactions can be made: “water only,” in which only the enzyme and water are boiled; and “all inside,” in which all the reaction components (enzyme, liquid, buffer, dNTPs, genomic DNA and primers) are boiled.

Following boiling, any additional reaction component that is required can be added to PCR tubes and an ordinary PCR program can be set with about 30 cycles.

It is expected that the enriched nanostructure composition of the present embodiments will protect the enzyme from heating, both under conditions where all the components are subjected to heat stress and where only the enzyme are subjected to heat stress.

### Example 32

### Stabilizing Effect

This Example describes another prophetic experiment for investigating the effect of the enriched nanostructure composition of the present embodiments on the stability of a protein. To this end, experiments with Peq-lab and Bio-lab are described.

The enriched nanostructure composition of the present embodiments is interchangeable referred to in this example as “gas enriched nanostructure composition” abbreviated “GENC.”

PCR reactions can be set as follows:

### Peq-Lab Samples

20.4 μl of either GENC or distilled water (Reverse Osmosis, RO)

0.1 μl Taq polymerase (Peg-lab, Taq DNA polymerase, 5 U/μl)

Samples can be set up and placed in a PCR machine at a constant temperature of 95° C. Incubation time can be: 60, 75 or 90 minutes. Three samples can be prepared, one for each incubation time.

Following boiling of the Taq enzyme the following components can be added:

2.5 μl 10× reaction buffer Y (Peg-lab)

0.5 μl dNTPs 10 mM (Bio-lab)

1 μl primer GAPDH mix 10 pmol/μl

0.5 μl genomic DNA 35 μg/μl

### Biolab Samples

18.9 μl of either GENC or RO water.

0.1 μl Taq polymerase (Bio-lab, Taq polymerase, 5 U/μl)

Samples can be set up and placed in a PCR machine at a constant temperature of 95° C. Incubation time can be: 60, 75, 90 120 and 150 minutes. Five samples can be prepared, one for each incubation time.

Following boiling of the Taq enzyme the following components can be added:

2.5 μl TAQ 10× buffer Mg-free (Bio-lab)

1.5 μl MgCl₂ 25 mM (Bio-lab)

0.5 μl dNTPs 10 mM (Bio-lab)

1 μl primer GAPDH mix (10 pmol/μl)

0.5 μl genomic DNA (35 μg/μl)

For each treatment (RO water or GENC), a positive and negative control can be made. Positive control can be without boiling the enzyme, and negative control can be without boiling the enzyme and without DNA in the reaction. A PCR mix can be made for the boiled tag assays as well for the control reactions.

### PCR Program

(i) 94° C. 2 minutes denaturation

(ii) 94° C. 30 seconds denaturation

(iii) 60° C. 30 seconds annealing

(iv) 72° C. 30 seconds elongation

repeat steps (ii)-(iv) 30 times

(v) 72° C. 10 minutes elongation

It is expected that the enriched nanostructure composition of the present embodiments will protect both enzymes from heat stress.

### Example 33

### Heat Dehydrated Multiplex PCR Mix

This Example describes a prophetic experiment for investigating the applicability of the enriched nanostructure composition of the present embodiments in a multiplex PCR system.

The enriched nanostructure composition of the present embodiments is interchangeable referred to in this example as “gas enriched nanostructure composition” abbreviated “GENC.”

Standard PCR mixture can be prepared (for example, KCl buffer, dNTPs, Taq, BPB) which can also include the following ingredients:

Additives (final concentration): Sucrose (150 mM, 200 mM)

Taq enzyme: Biolab

Primers against Human Insulin Gene (internal control)

Human Genomic DNA (internal control)

The samples can be heat-dehydrated in an oven until GENC or the RO water is evaporated.

Rehydration can be performed with (A) only DDW (for RO water or GENC) and (B) EGD-Primers mix of PBFDV DNA segment, for RO water and GENC (multiplex)

It is expected that is will be possible to heat dehydrate a complete PCR mix and rehydrate it using the enriched nanostructure composition of the present embodiments while maintaining fidelity of reaction. This method may by used as an internal control for multiple purpose PCR reactions, a property that assures that the PCR reaction is performed correctly on a per sample basis (eliminating false negative results).

### Example 34

### Micro Volume PCR

This Example describes a prophetic experiment for investigating the applicability of the enriched nanostructure composition of the present embodiments in a small volume PCR reaction.

MVP can be performed at a final volume of 2 ul. The target DNA can be, for example, a plasmid comprising the PDX gene. A mix can be prepared and 2 ul of complete mix (containing both DNA, primer and the enriched nanostructure composition of the present embodiments) can be aliquoted into tubes and PCR can be performed.

It is expected that that the enriched nanostructure composition of the present embodiments will take part in a microreaction volume in PCR.

### Example 35

### Quantitative PCR

This Example describes a prophetic experiment for investigating the applicability of the enriched nanostructure composition of the present embodiments quantitative PCR reaction (QPCR). QPCR can be performed with Syber Green against several DNA targets (plasmid and genomic) and gene targets (Beta Actin, PDX, PCT etc.).

It is expected that QPCR of PDX plasmid with the enriched nanostructure composition of the present embodiments is proficient and utilizes amplification in an exponential manner (efficiency 101%, exponential slope) with no primer-dimer formations.

### Example 36

### Dispersion of Antiseptic Active Agents

This Example describes a prophetic experiment for investigating dispersion of antiseptic active agents in the enriched nanostructure composition of the present embodiments.

Strips comprising an antiseptic composition (comprising thymol, methyl salicylate, menthol and eucalyptol) can be dissolved in both the enriched nanostructure composition of the present embodiments and reverse osmosis water in order to compare their solvent properties.

### Materials

The enriched nanostructure composition of the present embodiments, RO water, Listerine™ (Pocket Pak) strips (Pfizer Consumer Healthcare, New Jersey).

### Method

A strip comprising an antiseptic composition can be removed from the package and cut in half. Each half can be placed in a vial with 5 ml of either the enriched nanostructure composition of the present embodiments or RO water. Both vials can be shaken for a few seconds and left to stand for a few minutes. The bottles can be visually inspected to ensure the strip halves are fully dissolved. OD can be measured at t=0 and t=2 hours using a USB 2000 Spectrophotometer (scan 180-850 nm).

It is expected that following an incubation, the antiseptic composition present in the strip created finer micelles over time, with more dispersion in the enriched nanostructure composition of the present embodiments compared with RO water. It is further expected that no or low phase separation will be apparent with the enriched nanostructure composition of the present embodiments.

### Example 37

### Hydrophobic Properties

This Example describes a prophetic experiment for investigating the hydrophobic properties of the enriched nanostructure composition of the present embodiments.

### Materials

The enriched nanostructure composition of the present embodiments, coloring agent (e.g., Phenol Bromide Blue), and a plastic apparatus such as the plastic apparatus described in International Patent Publication No. WO2007/077562, the contents of which are hereby incorporated by reference. The apparatus comprises an upper and lower chamber made from a hydrophobic plastic resin. The upper and lower chambers are molded such that very narrow channels which act as hydrophobic capillary channels interconnect the four upper chambers with the single lower chamber. These hydrophobic capillary channels simulate a typical membrane or other biological barriers.

### Method

The color mix can be diluted with the enriched nanostructure composition of the present embodiments or with water at a 1:1 dilution. A ten microlitre drop of the enriched nanostructure composition of the present embodiments+color composition can be placed in the four upper chambers of a first plastic apparatus. In parallel, a five hundred microlitre drop of the enriched nanostructure composition of the present embodiments can placed in the lower chamber of the plastic apparatus directly above the upper chambers.

Similarly, a ten microlitre drop of water+color composition can be placed in the four upper chambers, of a second plastic apparatus (similar to the first apparatus) whilst in parallel a five hundred microlitre drop of water can be placed in the lower chamber directly above the upper chambers. The location of the dye in each plastic apparatus can be analyzed fifteen minutes following placement of the drops.

It is expected that the lower chamber of the plastic apparatus comprising the water and color mix will be clear, and that the lower chamber of the plastic apparatus comprising the enriched nanostructure composition of the present embodiments and color mix will exhibit color. Such a result can indicate that the enriched nanostructure composition of the present embodiments comprises hydrophobic properties as it is able to flow through a hydrophobic capillary.

### Example 38

### Cryoprotection

This Example describes a prophetic experiment for investigating the effect of the enriched nanostructure composition of the present embodiments with standard cryoprotective solution on sperm quality post freezing and thawing.

Sperm motility can be measured under a light microscope, with the aid of a Helber small camera, by counting the number of motile sperm cells. Sperm viability can be measured by staining, e.g., Eosine Nigrozine staining. Sperm DNA fragmentation can be measured by Sperm Chromatin Structural Assay (SCSA). The ability of sperm to fertilize an egg can be measured by motile sperm organelle morphology examination (MSOM). MSOM examines the number of sperm cells with specific normal morphology and progressive motility, each shown in the literature to act as a marker for fertile cells.

The cryoprotective buffer can be a standard cryoprotective buffer (e.g., TES buffer) which comprises TRIS buffer, egg yolk and glycerol. Such cryoprotective buffer is commercially available from Irvine scientific, Santa Anna, Calif.

Sperm samples can be obtained from sub-fertile male volunteers and frozen, for example, in a PLANER KRYO-10 instrument using a gradual temperature reducing program. The specimens can be frozen either in the presence of TES (50% semen, 50% TES) or the enriched nanostructure composition of the present embodiments (50% semen, 25% TES and 25% of the enriched nanostructure composition). The frozen semen can be thawed after about two days for analysis. The protective effects of the two buffers following freezing on semen quality can be compared with a non-frozen native sample of the same semen. The experiment can be repeated plurality (e.g., three or more) times.

An improvement in sperm motility, viability and DNA fragmentation, with a higher percentage of normal cells surviving is expected for a freezing cocktail containing the enriched nanostructure composition of the present embodiments.

### Example 39

### Transformation Efficiencies in Electrocompetent Cells

This Example describes a prophetic experiment for investigating the effect of the enriched nanostructure composition of the present embodiments on transformation efficiencies in electrocompetent cells.

Electro-competent cells can be prepared according to a standard protocol in which the water component (H₂0) is substituted with the enriched nanostructure composition of the present embodiments at different steps and in different combinations.

E. Coli cells can be grown in rich media until the logarithmic phase and then harvested by centrifugation. This rich media has a rich nutrient base which provides amino acids, vitamins, inorganic and trace minerals at levels higher than those of LB Broth. The medium can be buffered at pH 7.1±0.2 with potassium phosphate to prevent a drop in pH and to provide a source of phosphate. These modifications permit higher cell yields than can be achieved with LB.

The pellets can be washed several (e.g., three) times in standard cold water and re-suspended in either water containing 10% glycerol (standard) or in the enriched nanostructure composition of the present embodiments containing 2, 5, or 10% glycerol and frozen at −80° C.

Electroporation can be performed under standard conditions using pUC plasmid DNA diluted in water and the bacteria can be plated on LB plates comprising antibiotic to for colony counting. Colonies can be counted the following day and transformation efficiency can be determined.

It is expected that resuspension of electrocompetent bacteria in the dilutions of the enriched nanostructure composition of the present embodiments will increase transformation efficiencies.

### Example 40

### DNA Uptake in Chemically Competent Cells

This Example describes a prophetic experiment for investigating the effect of the enriched nanostructure composition of the present embodiments on DNA uptake by different chemically competent cells was studied.

pUC plasmid DNA can be diluted 1:10 in either water or the enriched nanostructure composition of the present embodiments and can be used for transformation of bacteria strains XL1 Blue, for example using the heat shock method. Following incubation for ten minutes on ice, the DNA together with the bacteria can be incubated at 42° C. for 30 seconds and plated on LB plates comprising antibiotic for colony counting. Colonies can be counted the following day and transformation efficiency can be determined.

It is expected that dilution of DNA in the enriched nanostructure composition of the present embodiments will improve DNA uptake by competent cells.

### Example 41

### DNA Uptake in Human Cells

This Example describes a prophetic experiment for investigating the effect of the enriched nanostructure composition of the present embodiments on dna uptake in a primary human cell culture.

Human bone marrow primary cells can be grown in Mem-alpha 20% fetal calf serum and plated so that they are 80% confluent about 24 hours prior to cell culture. Cells can be transfected using a standard Lipofectamine 2000 (Invitrogen™) transfection procedure with a green fluorescent protein (GFP) construct. The transfection can be repeated using a mix of the enriched nanostructure composition of the present embodiments and 12.5% of the amount of Lipofectamine 2000 of a control experiment.

It is expected that the combination of the enriched nanostructure composition of the present embodiments and Lipofectamine 2000 will increase transfection efficiency in primary cells.

### Example 42

### Uptake of Antibiotic

This Example describes a prophetic experiment for investigating the effect of the enriched nanostructure composition of the present embodiments on colony uptake of antibiotic.

Bacterial colonies can be grown on peptone/agar plates in the presence and absence of antibiotic. To this end Bacillus subtilis bacterial colonies can be pre-grown in the presence and absence of the enriched nanostructure composition of the present embodiments and can subsequently be plated on 0.5% agar with 10 g/l peptone. Bacterial colonies can also be pre-grown in the presence of reverse osmosis-water mixed with the same source powder as that used in the preparation of the enriched nanostructure composition of the present embodiments and can subsequently be plated on 0.5% agar with 10 g/l peptone. T strain bacterial colonies can be pre-grown in the presence and absence of the enriched nanostructure composition of the present embodiments and can subsequently be plated on 1.75% agar with 5 g/l peptone (prepared using the enriched nanostructure composition of the present invention) both in the presence and absence of streptomycin at the same minimum inhibitory concentration (MIC).

It is expected that the bacterial colony will be larger in the presence of the enriched nanostructure composition of the present embodiments. It is also expected that the colonies will show a different pattern in the presence of the enriched nanostructure composition of the present embodiments compared to control plates. It is also expected that the enriched nanostructure composition of the present embodiments will lead to faster bacterial growth relative to reverse osmosis-water, and that reverse osmosis-water supplemented with powder will exhibit slower growth. It is also expected that the combination of the streptomycin and the enriched nanostructure composition of the present embodiments will diminish the size of the colony.

### Example 43

### Bacterial Growth and Luminescence

This Example describes a prophetic experiment for investigating the effect of the enriched nanostructure composition of the present embodiments on growth and photo-luminescence of bacteria.

Bioluminescent Vibrio Harveyi bacteria (e.g., BB120 strain) can be grown in either a medium comprising the enriched nanostructure composition of the present embodiments or a medium comprising distilled water. Luminescent and turbidity measurements can be made using a standard ELISA reader.

It is expected that the enriched nanostructure composition of the present embodiments will increase the growth of Vibrio bacteria and the expression of the luminescence gene.

### Example 44

### Skin Cream Uptake

This Example describes a prophetic experiment for investigating the effect of the enriched nanostructure composition of the present embodiments on skin cream uptake in-vivo.

Patients suffering from acne can be topically administered with a commercial skin cream (e.g., Clearasil, Alleon Pharmacy) in the presence and absence of the enriched nanostructure composition of the present embodiments. The dilution of the cream with the enriched nanostructure composition of the present embodiments can be at a 1:1 ratio.

The therapeutic benefit of the enriched nanostructure composition of the present embodiments to the skin cream can be measured by UV light Facial Stage (commercially available from Moritex, Japan). The skin cream can be applied to the patients once a day for several (e.g., three) days.

It is expected that the number of acne spots will declined rapidly following treatment with the combination of the skin cream and the enriched nanostructure composition of the present embodiments.

### Example 45

### Isolation of Human Hybridomas

This Example describes a prophetic experiment for investigating the effect of the enriched nanostructure composition of the present embodiments on the first stage of monoclonal antibody production-isolation of hybridomas.

### Reagents for Cell Growth

RPMI 1640 (commercially available as powder from Beit-HaEmek, Israel) can be reconstituted either in the enriched nanostructure composition of the present embodiments or in control water, purified by reverse osmosis. Following reconstitution, sodium bicarbonate can be added to the media according to the manufacturers' recommendation, and the pH can be adjusted to 7.4. The culture media can be supplemented with 10% fetal calf serum, L-glutamine (4 mM), penicillin (100 U/mL), streptomycin (0.1 mg/mL), MEM-vitamins (0.1 mM), non-essential amino acids (0.1 mM) and sodium pyruvate (1 mM), all commercially available from GIBCO BRL, Life Technologies. HCF is commercially available from OriGen. All the supplements can be bought in a liquid, water-based form and diluted into the test and control media. 8-Azaguanine, HT and HAT (commercially available from Sigma) can be reconstituted from powder form with either the enriched nanostructure composition of the present embodiments or control RPMI.

### Chemical Reagents

Powdered PBS (commercially available from GIBCO BRL, Life Technologies) can be reconstituted with either the enriched nanostructure composition of the present embodiments or control water. Flaked PEG-1500 (commercially available from Sigma) can be reconstituted with both forms of sterile PBS (50% w/v); the pH of the liquid PEG can be adjusted to about 7 and it can be filter-sterilized. Hanks balanced salt solution is commercially available from Beit-HaEmek. Carbonate-bicarbonate buffer (0.05 M, pH=9.6) for ELISA plate-coating, OPD (can be used in 0.4 mg/mL) and phosphate-citrate buffer (0.05 M, pH=5.0) are commercially available from Sigma.

### Antibodies

Goat anti-human IgM and HRP-conjugated goat anti-human IgM are commercially available from Jackson ImmunoResearch, and standard human IgM are commercially available from Sigma.

### Fusion

Human peripheral blood mononuclear cells (PBMC) and fusion partner (MFP-2) cells can be washed several (e.g., four) times in serum-free culture medium prior to mixing and pelleting. 300 μl of PEG-1500, pre-warmed to 37° C. can be added to the cell mixture (about 0.10-200×10⁶ cells) and can be incubated for 3 minutes with constant shaking. PEG can then be diluted out of the cell mixture with Hanks balanced salt solution and complete RPMI. Fetal calf serum (10%) and HT (×2) can be added to the resultant cell suspension. The hybridomas that are generated during this process can be cultured in a 96-well plate in complete RPMI with HAT selection. The screening of the supernatants for antibodies can begin when the hybridoma cells occupy approximately ¼ of the well.

### Sandwich ELISA

A sandwich ELISA can be used to screen hybridoma supernatants for IgM. A capturing antibody (e.g., goat anti-human IgM) can be prepared in a carbonate/bicarbonate buffer and applied to the 96-well plate in a concentration of 100 ng/100 μl/well. The plate can be incubated overnight at 4° C.

The following steps can be performed at room temperature. After 1 hour of blocking with 0.3% dry milk in PBS, the supernatants from the hybridomas can be added for duration of 1.5 hours. Human serum diluted 1:500 in PBS can be used as a positive control. Hybridoma growth medium can be used as a negative control. The secondary antibody (e.g., HRP-conjugated goat anti-human IgM) can be prepared in blocking solution at a concentration of 1:5000 and incubated for 1 hour. To produce a colorimetric reaction, the plates can be incubated with OPD in phosphate-citrate buffer, containing 0.03% H₂O₂. The color reaction can be stopped with 10% Hydrochloric acid after about 15 minutes. The reading and the recording of the reaction can be performed on the Multiscan-Ascent using the 492 nm wavelength filter.

It is expected that the enriched nanostructure composition of the present embodiments will enables more consistency in the production of hybridomas relative to the control, due to the stabilizing influence. It is also expected that the process of creating and isolating stable hybridoma clones that secrete human monoclonal antibodies will be enhanced in the enriched nanostructure composition of the present embodiments.

### Example 46

### Cloning of Human Hybridomas

The next step in monoclonal antibody production following isolation of a relevant hybridoma is stabilizing it by cloning. This Example describes a prophetic experiment for investigating the effect of the enriched nanostructure composition of the present embodiments on the cloning of human hybridomas.

### Cloning

Cloning of hybridomas can be performed according to standard protocols. A limited number (approximately 10⁴) of cells can be serially diluted across the top row of a 96 well dish and then the contents of the first row can be serially diluted down the remaining 8 rows. In this way, wells toward the right bottom of the plate tend to have single cells.

### Screening for IgM Content

A sandwich ELISA can be used to screen hybridoma supernatants for IgM. A capturing antibody (e.g., goat anti-human IgM) can be prepared in a carbonate bicarbonate buffer and applied on a 96-well plate in a concentration of 100 ng/100 μL/well. The plate can be incubated overnight at +4° C.

The following steps are preferably performed at room temperature. Following 1 hour of blocking with 0.3% dry milk in PBS, the supernatants from the hybridomas can be applied for 1.5 hours. Human serum diluted 1:500 in PBS can be used as a positive control. For a background and as a negative control hybridoma growth medium can be used. The secondary antibody (e.g., HRP-conjugated goat anti-human IgM) can be prepared in blocking solution at a concentration of 1:5000 and incubated for 1 hour. To produce colorimetric reaction, the plates can be incubated with OPD in phosphate-citrate buffer, containing 0.03% H₂O₂. The color reaction can be stopped with 10% Hydrochloric acid after 15 minutes. The reading and the recording of the reaction can be performed on the Multiscan-Ascent using a 492 nm wavelength filter.

Lack of statistically significant difference among the frequency of IgM-producing clones or the distributions of antibody amounts produced in each plate can indicate that the hybridomas clone in the enriched nanostructure composition of the present embodiments as in the control media with HCF. Lack of cloning in the control media without the addition of HCF, can indicate that the enriched nanostructure composition of the present embodiments creates an environment that enhances clonability of unstable hybridomas. Enhanced frequency of hybridoma recovery following fusion in the enriched nanostructure composition of the present embodiments can also indicate enhanced clonability.

It is expected that the enriched nanostructure composition of the present embodiments will improve the fusion process, by means of elevating the physical cell fusion efficiency or stabilizing the hybridomas created in the process of fusion.

### Example 47

### Proliferation

This Example describes a prophetic experiment for investigating the effect of the enriched nanostructure composition of the present embodiments on proliferation. This prophetic experiment can be performed on human Mesenchymal cells.

Proliferation of human mesenchymal stem cells can be examined in media based on RO water or the enriched nanostructure composition of the present embodiments.

### Medium Preparation

250 ml of MEM alpha medium can be prepared by addition of 2.5 g of MEM and 0.55 g of Na HCO3 either to RO water of the enriched nanostructure composition of the present embodiments.

### Cell Culture

The cells can be maintained in MEM a supplemented with 20% fetal calf serum, 100 u/ml penicillin and 1 mg/ml streptomycin (see, e.g., Colter et al., 2001, PNAS 98:7841-7845). Cells can be counted and diluted to the concentration of 500 cells per ml, in 2 types of MEM a medium; one based on RO water, and the other based on the enriched nanostructure composition of the present embodiments. Cells can be grown in a 96 well plate, 100 μl medium with 50 cells in each well. After 8 days, cell proliferation can be estimated by a crystal violet viability assay. The dye in this assays, stains DNA. Upon solubilization, the amount of the dye taken up by the monolayer can be quantified in a plate reader, at 590 nm.

It is expected that the enriched nanostructure composition of the present invention will increase the proliferation of cells.

### Example 48

### Isolation of Human Hybridomas

This Example describes another prophetic experiment for investigating the effect of the enriched nanostructure composition of the present embodiments on the isolation of human hybridomas.

### Materials

### Reagents for Cell Growth

Media and supplements for cell growth are commercially available from GIBCO BRL, Life Technologies. RPMI 1640 and DMEM can be obtained in powder form and reconstituted either in the enriched nanostructure composition of the present embodiments or in DI water. After reconstitution sodium bicarbonate can be added to the media according to the manufacturers' recommendation, and there can be no further adjustment of pH. Prior to use, the media can be filter-sterilized through a 0.22 μm filter (Millipore). For the growth of hybridoma cells, RPMI can be supplemented with 10% fetal calf serum, L-glutamine (4 mM), penicillin (100 U/mL), streptomycin (0.1 mg/mL), MEM-vitamins (0.1 mM), non-essential amino acids (0.1 mM) and sodium pyruvate (1 mM). All the supplements above can be provided in a liquid form and used as is from the manufacturer. Thus they can be diluted into the enriched nanostructure composition of the present embodiments or control water. The control water can be DI based media—18.2 mega ohm ultrapure deionized water (DI water, UHQ PS, ELGA Labwater). 8-Azaguanine, HT and HAT (commercially available from Sigma) can be reconstituted from powder form with the enriched nanostructure composition of the present embodiments or DI RPMI. DMEM used for human primary fibroblasts, and CHO cells growth can be supplemented with 10% fetal calf serum, L-glutamine (4 mM), penicillin (100 U/mL), streptomycin (0.1 mg/mL).

Hybridoma cloning factor is commercially available from BioVeris.

### Chemical Reagents

Powdered PBS is commercially available from GIBCO BRL, Life Technologies. PEG-1450 (P5402) is commercially available from Sigma and can be reconstituted with sterile PBS based on the enriched nanostructure composition of the 5. present embodiments or on control water (50% w/v). The preparation can be adjusted to pH 7.2, DMSO (v/v) (commercially available from Sigma) can be added to 10% followed by sterile filtration of the PEG solution through a 0.45 μm filter (commercially available from Millipore). Hanks balanced salt solution is commercially available from Biological Industries Beit-HaEmek LTD, Israel and can be used as is for the enriched nanostructure composition of the present embodiments and the control. Carbonate-bicarbonate buffer (0.05 M, pH=9.6) for ELISA plate-coating, OPD (used in 0.4 mg/mL) and phosphate-citrate buffer (0.05 M, pH=5.0) are commercially available from Sigma.

### Antibodies

Goat anti-human IgM/IgG and HRP-conjugated goat anti-human IgM/IgG; are commercially available from Jackson ImmunoResearch. Standard human IgM/IgG is commercially available from Sigma.

### Cells

MFP-2, CHO and primary human fibroblasts can be maintained for a week in a medium containing the enriched nanostructure composition of the present embodiments and a control medium so that the cells can be adapted to the media prior to experimentation. In addition, the fusion partner cell line MFP-2 can be maintained in RPMI 1640 with the addition of fetal bovine serum and additives along with 8-azaguanine to maintain the HGPRT minus phenotype. Primary human fibroblasts can be obtained from the ATCC and maintained in DMEM. The CHO cell line can be maintained in DMEM. All cell culture can be performed in complete media, which consists of culture media with the addition of fetal calf serum, glutamine and penicillin/streptomycin. For the MFP-2 cell line vitamins, nonessential amino acids and pyruvate can be added in complete medium.

### Methods

### Cell Fusion

The chemical fusion technique [Kohler G, Milstein C (1975) Nature 256: 495-497] with PEG 1450 which acts as a fusogen for creation of hybridomas with human peripheral blood lymphocytes, can be employed. PEG 1450 is typically prepared in PBS with the addition of 10% DMSO. For these experiments, the enriched nanostructure composition of the present embodiments can be used to prepare PBS, which can be used to make a PEG/DMSO solution; as a control preparation PEG can be prepared in control water based PBS. For fusion experiments comparing the enriched nanostructure composition of the present embodiments to control water, the reagents can be prepared in either the enriched nanostructure composition of the present embodiments or control water except for fetal bovine serum and concentrates of supplements. In addition, dilution of cells in Hanks balanced salts (HBSS) (see below), following fusion with PEG-1450, can be performed with a liquid form of HBSS (commercially available from Beit HaEmek, Israel) and used as is from the manufacturer.

For production of hybridomas, human peripheral blood mononuclear cells (PBMC) can be isolated from 40 mL of freshly drawn whole blood, purified with Histopaque 1077 (commercially available from Sigma), and washed 4 times in control water based culture medium without serum. The MFP-2 fusion partner cells can be either grown in a medium based on the enriched nanostructure composition of the present embodiments or a medium based on control water and then washed with the respective medium several (e.g., 4) times without serum. For each experiment a single batch of PBMC can be divided into two equal fractions, one of which is, used for the enriched nanostructure composition of the present embodiments and the other is used for control water fusions. Next, MFP-2 and PBMC can be mixed in a medium based on the enriched nanostructure composition of the present embodiments or a medium based on control water without serum and pelleted. PEG-1450 pre-warmed to 37° C. can be then added at 300 μL for 10-200×10⁶ of mixed cells. The cell mixture can be incubated with PEG for several (e.g., 3) minutes with constant shaking. PEG can be diluted out of the cell mixture with Hanks balanced salt solution and complete RPMI (prepared in either the enriched nanostructure composition of the present embodiments or control water). To the resultant cell suspension can be added: fetal calf serum (10%) and HT (×2). The hybridomas that are generated in this process can be cultured in 96-well plates (cell density—2×10⁶ lymphocytes/well) in complete RPMI with HAT selection. The screening of the supernatants for immunoglobulin production can be performed after the hybridoma cells occupied approximately ¼ of the well.

### Sandwich ELISA

A sandwich ELISA can be used to screen hybridoma supernatants for IgM/IgG. A capturing antibody (e.g., goat anti-human IgM/IgG) can be prepared in a carbonate/bicarbonate buffer and applied on a 96-well plate in a concentration of 100 ng/100 μL/well. The plate can be incubated overnight at 4° C.

The following steps are preferably performed at room temperature.

After 1 hour of blocking with 0.3% dry milk in PBS, the supernatants from the hybridomas can be applied for 1.5 hours. Human serum diluted 1:500 in PBS can be used as a positive control. For a background and as a negative control hybridoma growth medium can be used. The secondary antibody (e.g., HRP-conjugated goat anti-human IgM/IgG) can be prepared in blocking solution at a concentration of 1:5000 and incubated for 1 hour. To produce a colorimetric reaction the plates can be incubated with OPD in phosphate-citrate buffer, containing 0.03% H₂O₂. The color reaction can be stopped with 10% HCl after 15 minutes. The reading and the recording of the reaction can be performed with a Multiscan-Ascent (commercially available from Thermo Scientific) ELISA reader using a 492 nm wavelength filter. All reagents can be standard with the exception of the sandwich layer, which is preferably consisted of the enriched nanostructure composition of the present embodiments or DI based hybridoma supernatant.

### Cloning

Two hundred cells of a chosen clone can be diluted in a volume of 10 mL of media and seeded in a 96-well plate (100 μL/well), so that on average the wells contained 1-2 cells. The cells can be incubated and periodically fed and microscopically monitored for clonal growth. Clone's supernatant can be analyzed once it occupies ¼-½ of the well. The efficiency of cloning can be expressed in a number of viable clones per plate. Ten percent HCF (hybridoma cloning factor) can be added according to the experimental design.

### Cell Growth Assay

Growth of primary and immortalized cell lines can be monitored with a crystal violet dye retention assay. A fixed number of cells can be seeded in 96-well plates in multiple repeats. Cell growth can be stopped by fixation in 4% formaldehyde. Fixed cells can be stained with 0.5% crystal violet followed by extensive washing with water. The retained dye can be extracted in 100 μL/well of 0.1 M sodium citrate in 50% ethanol (v/v). The absorbance of the wells can be read at 550 nm, for example, using a Multiscan-Ascent microplate reader and the appropriate filter.

### Primary Human Fibroblast Culture

Starting at about passage twenty, human fibroblasts can be cultured and passed every week as long as the cells display typical fibroblast morphology and their number does not drop below the initially seeded amount. The number of passages and the number of calculations of population doublings can be recorded. The morphology and viability of the cells can be monitored microscopically.

### Data Analysis

The statistical significance of difference in the efficiency of fusion and cloning between experiments with the enriched nanostructure composition of the present embodiments and control experiments can be determined by the Chi-square test. The results of the growth test with primary human fibroblasts can be analyzed by an unpaired Students' t-test. Statistical p-values <0.05 can be considered significant.

The following results are expected in the presence of the enriched nanostructure composition of the present embodiments: enhance efficiency of hybridoma formation for production of human monoclonal antibodies, increased yield of hybridoma subclones, increased secretion of monoclonal antibodies from hybridomas, enhanced cell proliferation, faster growth of immortal cell lines and slower growth of primary human fibroblasts.

### Example 49

### Stem Cells Growth

MSCs are auto/paracrine cells [Caplan and Dennis 2006, J Cell Biochem 98(5): 1076-84], known to secrete factors that influence themselves and their surrounding cells. Gregory et al., [Gregory, Singh et al. 2003, J Biol Chem. 2003 Jul. 25; 278(30):28067-78. Epub 2003 May 9] have shown that cultured MSCs at 5 cells per cm² secrete dickkpof1 (DKK1) of the Wnt signaling pathway which enhance their proliferation. A similar effect can be achieved by adding 20% media from highly proliferating cells seeded at very low densities.

This Example describes a prophetic experiment for investigating the effect of the enriched nanostructure composition of the present embodiments on the growth of mesenchymal stem cells (MSCs)

### Cell Culture

Human bone marrow (BM) cells can be obtained from adult donors under approved protocols. They can be cultured as follows. Culture medium components are commercially available from Biological Industries, Beth Haemek, Israel.

10-ml BM aspirates can be taken from the iliac crust of male and female donors between the ages of 19-70. Mononuclear cells can be isolated using a density gradient (ficoll/paque, commercially available from Sigma) and resuspended in αMEM medium containing 25 mM glucose and supplemented with 16% FBS (lot no. CPB0183, commercially available from Hyclone, Logan, Utah), 100 units/ml penicillin, 100 mg/ml streptomycin, and 2 mM L-glutamine. Cells can be plated in 10-cm culture dishes (commercially available from Corning, N.Y.), and incubated at 37° C. with 5% humidified CO₂. After 24 hours, nonadherent cells can be discarded, and adherent cells can be thoroughly washed twice with PBS. The cells can be incubated for 5 to 7 days, harvested by treatment with 0.25 trypsin and 1 mM EDTA for 5 min at 37° C., seeded at 50-100 cells per cm² and cultured to confluence.

Following the above passage cells can be seeded in 24 well plates in densities of 50-100 cells per cm² and cultured in media based on the enriched nanostructure composition of the present embodiments or RO water, which can be prepared out of powdered media (e.g., 01-055-1A, commercially available from Biological Industries, Beth Haemek, Israel). The cell viability can be assayed via crystal violet assay, once every 5 days for a total of 20 days. Cells from one of the donor's can be seeded in the above densities in 6 well plates (triplicates) and the cells can be counted using a hemocytometer.

It is expected that the growth rate of MSC's in the enriched nanostructure composition of the present embodiments will be enhanced at least at low cell density.

### Example 50

### Electrochemiluminescent Reaction

This Example describes a prophetic experiment for investigating the effect of the enriched nanostructure composition of the present embodiments on electrochemiluminescent (ECL) detection system. The procedure can include detection of an HRP-conjugated secondary antibody using an immunoperoxidase ECL detection system in the presence and absence of the enriched nanostructure composition of the present embodiments.

### Preparation of ECL Reagents:

Stock A

(i) 50 μl of 250 mM Luminol (Sigma C-9008) in DMSO (Fluca 0-9253).

(ii) 22 μl of 90 mM p-Coumaric acid (Sigma C-9008) in DMSO.

(iii) 0.5 ml Tris 1M, pH 8.5.

(iv) 4.428 ml H₂O (total of 5 ml).

Stock B

(i) 3 μl H₂O₂.

(ii) 0.5 ml Tris 1M, pH 8.5.

(iii) 4.5 ml H₂O (total of 5 ml).

The following sources of ECL reagents can be used.

Standard; Ver 1.0, in which the dH₂O is replaced for all the reagents and buffers with the enriched nanostructure composition of the present embodiments; and Ver 1.1, in which the dH₂O of the reaction volume is replaced with the enriched nanostructure composition of the present embodiments.

Whole cell protein extract can be generated from Jurkat cells. The protein extract can be subjected to SDS-PAGE followed by protein blotting onto a nitrocellulose membrane. An antibody specific for ZAP70 protein (home made polyclonal serum Ab) can be incubated with the membrane at a dilution of 1:30000 (regular working dilution 1:3000). The antibody immunoreactive protein bands can be visualized by reaction with HRP-conjugated secondary antibody followed by development with an immunoperoxidase ECL detection system. An equal volume of stock A and stock B can be combined and the detection mix can be equilibrated for 5 minutes. The detection mix can be added directly to the blot (protein side up) and incubated about 3 minutes at room temperature. An x-ray film can be exposed to the nitrocellulose membrane for 1 minute, 5 minutes and 10 minutes.

It is expected that the replacement of the water with the enriched nanostructure composition of the present embodiments will increase the sensitivity of the ECL reaction.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

The teachings of International Patent Publication Nos. WO2007/077562, WO2007/077560, WO2007/077561, WO2007/077563, WO2008/081456 and WO2008/081455, are excluded from the scope of some embodiments of the present invention.

1. A method of producing a nanostructure composition from a solid powder, the method comprising:
(a) heating the solid powder, thereby providing a heated solid powder;
(b) immersing said heated solid powder in a liquid in the presence of a gas medium, said liquid being colder than said heated powder; and
(c) irradiating said cold liquid, said heated solid powder and said gas medium by electromagnetic radiation selected such that nanostructures are formed from particles of the solid powder and a stable gas phase is formed from the gas medium.

2. The method of claim 1, further comprising passing said heated solid powder through said gas medium prior to said immersion so as to establish said presence of said gas medium.

3. The method of claim 1, further comprising introducing said gas medium into said liquid prior to said immersion so as to establish said presence of said gas medium.

4. The method of claim 1, wherein said gas medium comprises a hydrophobic gas.

5. The method of claim 1, wherein said gas medium is selected from the group consisting of carbon dioxide, oxygen, nitrogen, sulfur dioxide, hydrogen, fluorine, methane, hexane, hexafluoroethane and air.

6. The method of claim 1, wherein the solid powder comprises micro-sized particles.

7. The method of claim 6, wherein said micro-sized particles are crystalline particles.

8. The method of claim 7, wherein said nanostructures are crystalline nanostructures.

9. The method of claim 1, wherein said liquid comprises water.

10. The method of claim 1, wherein the solid powder is selected from the group consisting of a ferroelectric material and a ferromagnetic material.

11. The method of claim 1, wherein the solid powder is selected from the group consisting of BaTiO₃ and WO₃.

12. The method of claim 1, wherein the solid powder comprises hydroxyapatite.

13. The method of claim 1, wherein the solid powder comprises a material selected from the group consisting of a mineral, a ceramic material, glass, metal and synthetic polymer.

14. The method of claim 1, wherein said electromagnetic radiation is in the radiofrequency range.

15. The method of claim 14, wherein said electromagnetic radiation is continues wave electromagnetic radiation.

16. The method of claim 14, wherein said electromagnetic radiation is modulated electromagnetic radiation.

17. A nanostructure composition comprising a liquid, nanostructures and a stable or meta-stable gas phase, wherein at least one of said nanostructures has a core material of a nanometric size and an envelope of ordered fluid molecules being in a steady physical state with said core material.

18. The nanostructure composition of claim 17, being capable of releasing said gas in response to excitation energy applied thereto and collecting said gas when said excitation energy is terminated.

19. The nanostructure composition of claim 17, being prepared in non-atmospheric conditions.

20. The nanostructure composition of claim 17, being prepared in the presence of a gas jet.

21. The nanostructure composition of claim 17, being prepared in the presence of gas at a concentration which is substantially different from natural atmospheric concentration of said gas.

22. The nanostructure composition of claim 17, being prepared in the presence of gas at a temperature which is substantially below an ambient temperature.

23. The nanostructure composition of claim 17, wherein said envelope of fluid molecules is distinguishable from said liquid.

24. The nanostructure composition of claim 17, wherein said core material is crystalline.

25. The nanostructure composition of claim 17, wherein said liquid comprises water.

26. The nanostructure composition of claim 17, wherein said gas phase comprises a hydrophobic gas.

27. The nanostructure composition of claim 17, wherein said gas phase is selected from the group consisting of carbon dioxide, oxygen, nitrogen, sulfur dioxide, hydrogen, fluorine, methane, hexane, hexafluoroethane and air.

28. The nanostructure composition of claim 17, wherein said gas phase resides in or is attached to said envelope.

29. The nanostructure composition of claim 17, wherein said gas phase resides in or is attached to said core.

30. The nanostructure composition of claim 17, wherein said gas phase resides in liquid regions between said nanostructures.

31. The nanostructure composition of claim 17, wherein when the nanostructure composition is first contacted with a surface and then washed by a predetermined wash protocol, an electrochemical signature of the composition is preserved on said surface.

32. The nanostructure composition of claim 17, wherein the nanostructure composition is characterized by a zeta potential which is substantial larger than a zeta potential of said liquid per se.

33. The nanostructure composition of claim 17, wherein said nanostructure has a specific gravity which is lower than or equal to a specific gravity of said liquid.

34. The nanostructure composition of claim 17, wherein when the nanostructure composition is mixed with a dyed solution, spectral properties of said dyed solution are substantially changed.

35. The nanostructure composition of claim 17, wherein the nanostructure composition is characterized by an enhanced ultrasonic velocity relative to water.

36. The nanostructure composition of claim 17, wherein the nanostructure composition is capable of facilitating increment of bacterial colony expansion rate.

37. The nanostructure composition of claim 17, wherein the nanostructure composition is capable of facilitating increment of phage-bacteria or virus-cell interaction.

38. The nanostructure composition of claim 17, wherein the nanostructure composition is capable of enhancing macromolecule binding to solid phase matrix.

39. The nanostructure composition of claim 17, wherein the nanostructure composition is capable of at least partially de-folding DNA molecules.

40. The nanostructure composition of claim 17, wherein the nanostructure composition is capable of stabilizing enzyme activity.

41. The nanostructure composition of claim 17, wherein the nanostructure composition is capable of altering bacterial adherence to biomaterial.

42. The nanostructure composition of claim 17, wherein the nanostructure composition is capable of improving affinity of binding of nucleic acids to a resin and improving gel electrophoresis separation.

43. The nanostructure composition of claim 17, wherein the nanostructure composition is capable of increasing a capacity of a column.

44. The nanostructure composition of claim 17, wherein the nanostructure composition is characterized by an enhanced ability to dissolve or disperse a substance relative to water.

45. The nanostructure composition of claim 17, wherein the nanostructure composition is characterized by an enhanced buffering capacity relative to water.

46. The nanostructure composition of claim 17, wherein the nanostructure composition is capable of improving efficiency of nucleic acid amplification process.

47. A kit for polymerase chain reaction, comprising, in separate packaging:
(a) a thermostable DNA polymerase; and
(b) the nanostructure composition of claim 17.

48. A method of amplifying a DNA sequence, the method comprising:
(a) providing the nanostructure composition of claim 17; and
(b) in the presence of said nanostructure composition, executing a plurality of polymerase chain reaction cycles on the DNA sequence, thereby amplifying the DNA sequence.

49. The nanostructure composition of claim 17, wherein the nanostructure composition is capable of improving efficiency of real-time polymerase chain reaction.

50. A kit for real-time polymerase chain reaction, comprising:
(a) a thermostable DNA polymerase;
(b) a double-stranded DNA detecting molecule; and
(c) the nanostructure composition of claim 17.

51. The nanostructure composition of claim 17, wherein the nanostructure composition is capable of allowing the manipulation of at least one macromolecule in the presence of a solid support.

52. An antiseptic composition comprising at least one antiseptic agent and the nanostructure composition of claim 17.

53. A method of disinfecting a body surface of an individual comprising providing to an individual in need thereof an antiseptic effective amount of a composition wherein said composition comprises the nanostructure composition of claim 17, thereby disinfecting a body surface of an individual.

54. A method of sterilizing an object comprising contacting the object with a composition which comprises the nanostructure composition of claim 17, thereby sterilizing the object.

55. A cryoprotective composition comprising the nanostructure composition of claim 17, and at least one cryoprotective agent.

56. A method of cryopreserving cellular matter comprising:
(a) contacting the cellular matter with the nanostructure composition of claim 17; and
(b) subjecting the cellular matter to a cryopreserving temperature,
thereby cryopreserving the cellular matter.

57. A cryopreservation container comprising the cryoprotective composition of claim 55.

58. A pharmaceutical composition comprising:
(a) at least one pharmaceutical agent as an active ingredient;
(b) the nanostructure composition of claim 17, being formulated to enhance in vivo uptake of said at least one pharmaceutical agent.

59. A method of enhancing in vivo uptake of a pharmaceutical agent into a cell comprising administering the pharmaceutical composition of claim 58 to an individual, thereby enhancing in vivo uptake of the pharmaceutical agent into the cell.

60. A method of cell-fusion, the method comprising fusing cells in a medium comprising the nanostructure composition of claim 17, thereby fusing cells.

61. A method of culturing eukaryotic cells, the method comprising incubating the cells in a medium comprising the nanostructure composition of claim 17, thereby culturing eukaryotic cells.

62. A cell culture medium comprising a eukaryotic cell culture medium and the nanostructure composition of claim 17.

63. An article of manufacture comprising: a packaging material and the nanostructure composition of claim 17 for culturing of eukaryotic cells being contained within said packaging material.

64. An article of manufacture comprising: a packaging material and the nanostructure composition of claim 17 for generating monoclonal antibodies being contained within said packaging material.

65. A method of generating a monoclonal antibody, the method comprising fusing an immortalizing cell with an antibody producing cell to obtain a hybridoma in a medium comprising the nanostructure composition of claim 17.

66. A method of dissolving or dispersing cephalosporin comprising contacting cephalosporin with the nanostructure composition of claim 17 under conditions allowing dispersion or dissolving of cephalosporin.

67. A kit for detecting an analyte comprising:
(a) a detectable agent; and
(b) the nanostructure composition of claim 17.

68. An article of manufacture comprising: a packaging material and the nanostructure composition of claim 17 for enhancing detection of a detectable moiety being contained within said packaging material.

69. Apparatus for recycling gas, comprising a nano structure composition and an excitation device for exciting said nanostructure composition, said nanostructure composition being capable of releasing gas when said excitation device is active, and collecting said gas upon deactivation of said excitation device.

70. Apparatus for attracting insects, comprising the apparatus of claim 69.

71. Apparatus for enhancing plant growth comprising the apparatus of claim 69.

72. A method of attracting insects, comprising activating the excitation device of the apparatus of claim 69, thereby attracting the insects.

73. A method of enhancing plant growth, comprising activating the excitation device of the apparatus of claim 69 during daylight hours, thereby enhancing plant growth.

74. The nanostructure composition of claim 17, wherein a concentration of said nanostructures is lower than 10²⁰ nanostructures per litter.

75. The nanostructure composition of claim 17, wherein a concentration of said nanostructures is lower than 10¹⁵ nanostructures per litter.
